(19) United States
(12) Patent Application Publication
(10) Pub. No.: US 2009/0247544 A1
(43) Pub. Date: Oct. 1, 2009

(54) Certain Chemical Entities, Compositions and Methods

(75) Inventors: Bradley P. MORGAN, Moraga, CA (US); Fady MALIK, Burlingame, CA (US); David J. MORGANS, JR., Los Altos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Cytokinetics, Inc.

(21) Appl. No.: 12/371,499

(22) Filed: Feb. 13, 2009

Related U.S. Application Data
(63) Continuation of application No. 11/639,400, filed on Dec. 13, 2006, now abandoned.
(60) Provisional application No. 60/751,032, filed on Dec. 15, 2005.

(51) Int. Cl. A61K 31/497

(52) U.S. Cl. 514/253.11; 514/254.04

(57) ABSTRACT

Certain substituted urea derivatives modulate diskeletal myosin, skeletal actin, skeletal tropomyosin, skeletal troponin C, skeletal troponin I, skeletal troponin T, and skeletal muscle, including fragments and isoforms thereof, as well as the skeletal sarcomere, and are useful in the treatment of obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions.

This application claims the benefit of U.S. Provisional Patent Application No. 60/751,032, filed Dec. 15, 2005, which is incorporated herein by reference for all purposes.

The invention relates to certain substituted urea derivatives, particularly to certain chemical entities that modulate diskeletal myosin, skeletal actin, skeletal tropomyosin, skeletal troponin C, skeletal troponin I, skeletal troponin T, and skeletal muscle, including fragments and isoforms thereof, as well as the skeletal sarcomere, and specifically to chemical entities, pharmaceutical compositions and methods of treatment one or more of obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions.

The cytoskeleton of skeletal and cardiac muscle cells is unique compared to that of all other cells. It consists of a nearly crystalline array of closely packed cytoskeletal proteins called the sarcomere. The sarcomere is elegantly organized as an interdigitating array of thin and thick filaments. The thick filaments are composed of myosin, the motor protein responsible for transducing the chemical energy of ATP hydrolysis into force and directed movement. The thin filaments are composed of actin monomers arranged in a helical array. There are four regulatory proteins bound to the actin filaments, which allows the contraction to be modulated by calcium ions. An influx of intracellular calcium initiates muscle contraction; thick and thin filaments slide past each other driven by repetitive interactions of the myosin motor domains with the thin actin filaments.

Myosin is the most extensively studied of all the motor proteins. Of the thirteen distinct classes of myosin in human cells, the myosin-II class is responsible for contraction of skeletal, cardiac, and smooth muscle. This class of myosin is significantly different in amino acid composition and in overall structure from myosin in the other twelve distinct classes. Myosin-II consists of two globular head domains linked together by a long alpha-helical coiled-coiled tail that assembles with other myosin-IIs to form the core of the sarcomere's thick filament. The globular heads have a catalytic domain where the actin binding and ATP functions of myosin take place. Once bound to an actin filament, the release of phosphate (cf. ATP to ADP) leads to a change in structural conformation of the catalytic domain that in turn alters the orientation of the light-chain binding lever arm domain that extends from the globular head; this movement is termed the powerstroke. This change in orientation of the myosin head in relationship to actin causes the thick filament of which it is a part to move with respect to the thin actin filament to which it is bound. Un-binding of the globular head from the actin filament (also Ca²⁺ modulated) coupled with return of the catalytic domain and light chain to their starting conformation/orientation completes the contraction and relaxation cycle, responsible for intracellular movement and muscle contraction.

Tropomyosin and troponin mediate the calcium effect on the interaction on actin and myosin. The skeletal troponin complex regulates the action of several actin units at once, and is comprised of three polypeptide chains: skeletal troponin C, which binds calcium ions; troponin I, which binds to actin; and troponin T, which binds to tropomyosin.

Abnormal contraction of skeletal muscle is thought to be a pathogenetic cause of several disorders, including obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions, which pose serious health problems as adult diseases. The contraction and relaxation of skeletal muscle are mainly controlled by increases and decreases of intracellular calcium. Intracellular calcium is thought to bind with calmodulin to activate myosin light chain phosphorylation enzyme. According to the myosin phosphorylation theory, this activation results in phosphorylation of the myosin light chain, causing contraction of skeletal muscles. Following this theory, various calcium antagonists have been developed which reduce intracellular calcium and distend blood vessels.

However, in recent years, a calcium sensitivity reinforcing mechanism has been proposed, as a sustained smooth muscle contraction of blood vessel, trachea and the like is inexplicable by the myosin phosphorylation theory alone. A new theory has developed with a contraction mechanism independent of intracellular calcium level.

Therefore, pharmaceutical agents which only reduce intracellular calcium are insufficient to treat diseases caused by abnormal skeletal muscle contraction. Accordingly, there is a need for the development of new compounds modulate skeletal muscle. There remains a need for agents that exploit new mechanisms of action and which may have better outcomes in terms of relief of symptoms, safety, and patient mortality, both short-term and long-term and an improved therapeutic index. The present invention provides such agents; compositions; methods of treating obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions; and uses thereof.

Many factors may cause obesity, several of which have a variable genetic component. Some types of obesity are caused by single-gene mutations, while some types are caused by various diseases (such as damage to the ventromedial hypothalamus) in individuals whom otherwise would not be obese. The morbidity and mortality associated with being obese are common knowledge. Obesity is treatable with diet, exercise, and behavior modification. Drug therapy is also used, but the potential for abuse, side effects, and efficacy of the currently available pharmaceuticals is of considerable concern.

Sarcopenia is believed to be primarily due to disuse atrophy of the skeletal muscle fibers, but it is possible that age-associated changes in myofibrillar protein metabolism, nutritional status, neuromuscular function, and tissue responsiveness to trophic factors may also play a role. Medical intervention to prevent, treat or reverse sarcopenia is extremely limited, but current therapies include androgen and estrogen replacement therapies.

Wasting syndrome is associated with old age and AIDS, and typically involves the loss of skeletal muscle mass. Therapies include improved diet, human growth hormone, and treatment of AIDS (in patients with AIDS), but a satisfactory cure has not been found.

Frailty, common in the every old, is a condition characterized by impaired strength, endurance, and balance, vulnerability to trauma and other stressors, and high risk for morbidity, disability, and mortality. Inflammatory, musculoskeletal, cardiorespiratory, metabolic, hematologic, neurologic, immunologic and endocrine functions are thought to contribute to frailty, but few have been studied.

Muscle spasm may be caused by a myriad of factors, including inactivity, a pinched nerve, muscle fatigue, heavy exercise, dehydration, pregnancy, hypothyroidism, depleted magnesium or calcium stores and other metabolic abnormalities, alcoholism and kidney failure leading to uremia. Stretching the muscle may relieve muscle spasm, but drug therapy is not generally used.

Most neuromuscular diseases are incurable. Rehabilitation programs help maintain neuromuscular disease patients' quality of life.

The present invention provides compounds that are believed to bind to and/or regulate the activity of diskeletal myosin, skeletal actin, skeletal tropomyosin, skeletal troponin C, skeletal troponin I, skeletal troponin T, and skeletal muscle, including fragments and isoforms thereof, and the skeletal sarcomere. Each present targets for the treatment of obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions, and thereby modulate contraction of skeletal muscle.

Provided are methods for treating a patient having a disease chosen from obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions, comprising administering to the patient a therapeutically effective amount of at least one chemical entity chosen from compounds of Formula I:

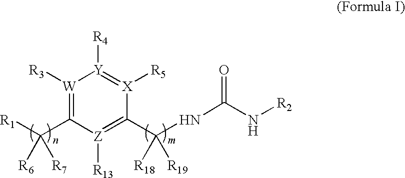

and pharmaceutically acceptable salts, chelates, non-covalent complexes, prodrugs, and mixtures thereof, wherein:
W, X, Y, and Z are independently —C═ or —N═, provided that no more than two of W, X, Y, and Z are —N═;
m is zero, one, two, or three;
n is one, two, or three;
R₁ is optionally substituted amino or optionally substituted heterocycloalkyl;
R₂ is optionally substituted aryl, optionally substituted aralkyl, optionally substituted cycloalkyl, optionally substituted heteroaryl, optionally substituted heteroaralkyl or optionally substituted heterocycloalkyl,
R₃ is hydrogen, halo, cyano, optionally substituted alkyl, optionally substituted heterocycloalkyl, optionally substituted alkoxy, or optionally substituted heteroaryl; or R₃ is absent when W is —N═;
R₄ is hydrogen, halo, cyano, optionally substituted alkyl, optionally substituted heterocycloalkyl, optionally substituted alkoxy, or optionally substituted heteroaryl; or R₄ is absent when Y is —N═; and
R₅ is hydrogen, halo, cyano, optionally substituted alkyl, optionally substituted heterocycloalkyl, optionally substituted alkoxy, or optionally substituted heteroaryl; or R₅ is absent when X is —N═;
R₆ and R₇ are independently hydrogen, aminocarbonyl, alkoxycarbonyl, optionally substituted alkyl or optionally substituted alkoxy; or R₆ and R₇, taken together with the carbon to which they are attached, form an optionally substituted 3- to 7-membered ring which optionally incorporates one or two additional heteroatoms chosen from N, O, and S in the ring;
R₁₃ is hydrogen, halo, cyano, hydroxyl, optionally substituted alkyl, optionally substituted heterocycloalkyl, optionally substituted alkoxy, or optionally substituted heteroaryl; or R₁₃ is absent when Z is —N═; and
R₁₈ and R₁₉ are independently hydrogen, aminocarbonyl, alkoxycarbonyl, optionally substituted alkyl or optionally substituted alkoxy, or R₁₈ and R₁₉, taken together with the carbon to which they are attached, form an optionally substituted 3- to 7-membered ring which optionally incorporates one or two additional heteroatoms chosen from N, O, and S in the ring; or
R₁₈ and R₁₉ are absent when m is zero.

Also provided are methods for treating a patient having a disease chosen from obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions, comprising administering to the patient a therapeutically effective amount of at least one chemical entity chosen from compounds of Formula II:

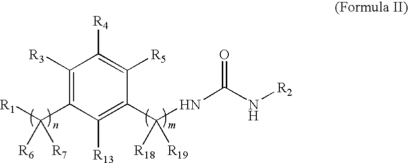

and pharmaceutically acceptable salts, chelates, non-covalent complexes, prodrugs, and mixtures thereof, wherein:
m is zero, one, two, or three;
n is one, two, or three;
R₁ is optionally substituted amino or optionally substituted heterocycloalkyl;
R₂ is optionally substituted aryl, optionally substituted aralkyl, optionally substituted cycloalkyl, optionally substituted heteroaryl, optionally substituted heteroaralkyl or optionally substituted heterocycloalkyl,
R₃ is hydrogen, halo, cyano, optionally substituted alkyl, optionally substituted heterocycloalkyl, optionally substituted alkoxy, or optionally substituted heteroaryl;
R₄ is hydrogen, halo, cyano, optionally substituted alkyl, optionally substituted heterocycloalkyl, optionally substituted alkoxy, or optionally substituted heteroaryl;
R₅ is hydrogen, halo, cyano, optionally substituted alkyl, optionally substituted heterocycloalkyl, optionally substituted alkoxy, or optionally substituted heteroaryl;
R₆ and R₇ are independently hydrogen, aminocarbonyl, alkoxycarbonyl, optionally substituted alkyl or optionally substituted alkoxy; or R₆ and R₇, taken together with the carbon to which they are attached, form an optionally substituted 3- to 7-membered ring which optionally incorporates one or two additional heteroatoms chosen from N, O, and S in the ring;
R₁₃ is hydrogen, halo, cyano, hydroxyl, optionally substituted alkyl, optionally substituted heterocycloalkyl, optionally substituted alkoxy, or optionally substituted heteroaryl; and
R₁₈ and R₁₉ are independently hydrogen, aminocarbonyl, alkoxycarbonyl, optionally substituted alkyl or optionally substituted alkoxy, or R₁₈ and R₁₉, taken together with the carbon to which they are attached, form an optionally substituted 3- to 7-membered ring which optionally incorporates one or two additional heteroatoms chosen from N, O, and S in the ring; or
R₁₈ and R₁₉ are absent when m is zero.

Also provided are methods for treating a patient having a disease chosen from obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions, comprising administering to the patient a therapeutically effective amount of at least one chemical entity chosen from compounds of Formula III:

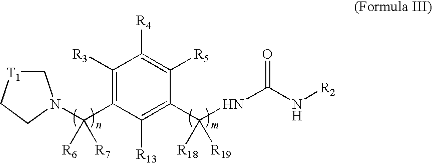

and pharmaceutically acceptable salts, chelates, non-covalent complexes, prodrugs, and mixtures thereof, wherein:
m is zero, one, two, or three;
n is one, two, or three;
T₁ is chosen from —CHR₁₄—, —NR₁₅CHR₁₄—, —CHR₁₄NR₁₅—, and —CHR₁₄CHR₁₄—;
R₂ is optionally substituted aryl, optionally substituted aralkyl, optionally substituted cycloalkyl, optionally substituted heteroaryl, optionally substituted heteroaralkyl or optionally substituted heterocycloalkyl,
R₃ is hydrogen, halo, cyano, optionally substituted alkyl, optionally substituted heterocycloalkyl, optionally substituted alkoxy, or optionally substituted heteroaryl;

R₄ is hydrogen, halo, cyano, optionally substituted alkyl, optionally substituted heterocycloalkyl, optionally substituted alkoxy, or optionally substituted heteroaryl;
R₅ is hydrogen, halo, cyano, optionally substituted alkyl, optionally substituted heterocycloalkyl, optionally substituted alkoxy, or optionally substituted heteroaryl;
R₆ and R₇ are independently hydrogen, aminocarbonyl, alkoxycarbonyl, optionally substituted alkyl or optionally substituted alkoxy; or R₆ and R₇, taken together with the carbon to which they are attached, form an optionally substituted 3- to 7-membered ring which optionally incorporates one or two additional heteroatoms chosen from N, O, and S in the ring;
R₁₃ is hydrogen, halo, cyano, hydroxyl, optionally substituted alkyl, optionally substituted heterocycloalkyl, optionally substituted alkoxy, or optionally substituted heteroaryl;
R₁₄ and R₁₅ is independently hydrogen, optionally substituted alkyl, optionally substituted acyl, carboxy, optionally substituted lower alkoxycarbonyl, optionally substituted aminocarbonyl, optionally substituted alkoxy, optionally substituted cycloalkoxy, optionally substituted sulfonyl, optionally substituted amino, optionally substituted cycloalkyl, or optionally substituted heterocycloalkyl, and
R₁₈ and R₁₉ are independently hydrogen, aminocarbonyl, alkoxycarbonyl, optionally substituted alkyl or optionally substituted alkoxy, or R₁₈ and R₁₉, taken together with the carbon to which they are attached, form an optionally substituted 3- to 7-membered ring which optionally incorporates one or two additional heteroatoms chosen from N, O, and S in the ring; or
R₁₈ and R₁₉ are absent when m is zero.

Also provided are methods for treating a patient having a disease chosen from obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions, comprising administering to the patient a therapeutically effective amount of at least one chemical entity chosen from compounds of Formula IV:

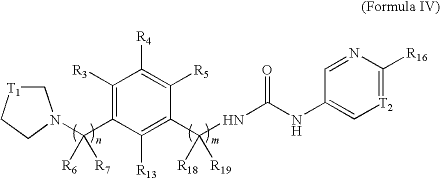

and pharmaceutically acceptable salts, chelates, non-covalent complexes, prodrugs, and mixtures thereof, wherein:
   T₁ is chosen from —CHR₁₄—, —NR₁₅CHR₁₄—, —CHR₁₄NR₁₅—, and —CHR₁₄CHR₁₄—;
   T₂ is —C═ or —N═;
   R₃ is hydrogen, halo, cyano, optionally substituted alkyl, optionally substituted heterocycloalkyl, optionally substituted alkoxy, or optionally substituted heteroaryl;

R₄ is hydrogen, halo, cyano, optionally substituted alkyl, optionally substituted heterocycloalkyl, optionally substituted alkoxy, or optionally substituted heteroaryl;
R₅ is hydrogen, halo, cyano, optionally substituted alkyl, optionally substituted heterocycloalkyl, optionally substituted alkoxy, or optionally substituted heteroaryl;
R₆ and R₇ are independently hydrogen, aminocarbonyl, alkoxycarbonyl, optionally substituted alkyl or optionally substituted alkoxy; or R₆ and R₇, taken together with the carbon to which they are attached, form an optionally substituted 3- to 7-membered ring which optionally incorporates one or two additional heteroatoms chosen from N, O, and S in the ring;
R₁₃ is hydrogen, halo, cyano, hydroxyl, optionally substituted alkyl, optionally substituted heterocycloalkyl, optionally substituted alkoxy, or optionally substituted heteroaryl;
R₁₄ and R₁₅ is independently hydrogen, optionally substituted alkyl, optionally substituted acyl, carboxy, optionally substituted lower alkoxycarbonyl, optionally substituted aminocarbonyl, optionally substituted alkoxy, optionally substituted cycloalkoxy, optionally substituted sulfonyl, optionally substituted amino, optionally substituted cycloalkyl, or optionally substituted heterocycloalkyl, and
R₁₆ is chosen from hydrogen, halo, cyano, optionally substituted acyl, optionally substituted alkyl, and optionally substituted alkoxy;

R₁₈ and R₁₉ are independently hydrogen, aminocarbonyl, alkoxycarbonyl, optionally substituted alkyl or optionally substituted alkoxy, or R₁₈ and R₁₉, taken together with the carbon to which they are attached, form an optionally substituted 3- to 7-membered ring which optionally incorporates one or two additional heteroatoms chosen from N, O, and S in the ring; or
R₁₈ and R₁₉ are absent when m is zero.

Provided is a method of treating one or more of obesity, sarcopenia, wasting syndrome, frailty, muscle spasm, cachexia, neuromuscular diseases (e.g., amyotrophic lateral sclerosis, spinal muscular atrophy, familial or acquired myopathies or muscular dystrophies), post-surgical and post-traumatic muscle weakness, and other conditions in a mammal which method comprises administering to a mammal in need thereof a therapeutically effective amount of at least one chemical entity described herein or a pharmaceutical composition comprising a pharmaceutically acceptable excipient, carrier or adjuvant and at least one chemical entity described herein.

Also provided is a method for treating a patient having a disease responsive to modulation of one or more of diskeletal myosin, skeletal actin, skeletal tropomyosin, skeletal troponin C, skeletal troponin I, skeletal troponin T, and skeletal muscle, including fragments and isoforms thereof, as well as the skeletal sarcomere in a mammal which method comprises administering to a mammal in need thereof a therapeutically effective amount of at least one chemical entity described herein or a pharmaceutical composition comprising a pharmaceutically acceptable excipient, carrier or adjuvant and at least one chemical entity described herein.

Also provided is a method for treating a patient having a disease responsive to potentiation of one or more of diskeletal myosin, skeletal actin, skeletal tropomyosin, skeletal troponin C, skeletal troponin I, skeletal troponin T, and skeletal muscle, including fragments and isoforms thereof, as well as the skeletal sarcomere in a mammal which method comprises administering to a mammal in need thereof a therapeutically effective amount of at least one chemical entity described herein or a pharmaceutical composition comprising a pharmaceutically acceptable excipient, carrier or adjuvant and at least one chemical entity described herein.

Also provided is a method for treating a patient having a disease responsive to inhibition of one or more of diskeletal myosin, skeletal actin, skeletal tropomyosin, skeletal troponin C, skeletal troponin I, skeletal troponin T, and skeletal muscle, including fragments and isoforms thereof, as well as the skeletal sarcomere in a mammal which method comprises administering to a mammal in need thereof a therapeutically effective amount of at least one chemical entity described herein or a pharmaceutical composition comprising a pharmaceutically acceptable excipient, carrier or adjuvant and at least one chemical entity described herein.

Other aspects and embodiments will be apparent to those skilled in the art from the following detailed description.

As used in the present specification, the following words and phrases are generally intended to have the meanings as set forth below, except to the extent that the context in which they are used indicates otherwise.

As used herein, “frailty” is a syndrome characterized by meeting three of the of the following five attributes: unintentional weight loss, muscle weakness, slow walking speed, exhaustion, and low physical activity. See Fried et al.; J Gerontol Med Sci; 2001; 56A (3): M146-M156, hereby incorporated by reference.

As used herein, “muscle spasm” means an involuntary contraction of a muscle. Muscle spasms may lead to cramps.

As used herein, “neuromuscular disease” means any disease that affects any part of the nerve and muscle. Neuromuscular disease encompasses critical illness polyneuropathy, prolonged neuromuscular blockade, acute myopathy as well as acute inflammatory demyelinating polyradiculoneuropathy, amyotrophic lateral sclerosis (ALS), autonomic neuropathy, Charcot-Marie-Tooth disease and other hereditary motor and sensory neuropathies, chronic inflammatory demyelinating polyradiculoneuropathy, dermatomyositis/polymyositis; diabetic neuropathy, dystrophinopathies, endocrine myopathies, focal muscular atrophies, hemifacial spasm, hereditary neuropathies of the Charcot-Marie-Tooth disease type, inclusion body myositis, Kennedy disease, Lambert-Eaton myasthenic syndrome, muscular dystrophy (e.g., limb-girdle, Duchenne, Becker, myotonic, facioscapulohumeral, etc.), metabolic myopathies, metabolic neuropathy, multifocal motor neuropathy with conduction blocks, myasthenia gravis, neuropathy of Friedreich Ataxia, neuropathy of leprosy, nutritional neuropathy, periodic paralyses, primary lateral sclerosis, restrictive lung disease, sarcoidosis and neuropathy, Schwartz-Jampel Syndrome, spinal muscle atrophy, stiff person syndrome, thyroid disease, traumatic peripheral nerve lesions, vasculitic neuropathy, among others. In certain embodiments, neuromuscular disease refers to amyotrophic lateral sclerosis, spinal muscular atrophy, familial or acquired myopathies or muscular dystrophies.

As used herein “obesity” means having a body mass index (BMI) greater than or equal to 30 kg/m². BMI is defined as weight (kg) divided by height (m²). Obesity encompasses hyperplastic obesity, an increase in the number of fat cells, and hypertrophic obesity, an increase in the size of the fat cells. Overweight is defined as having a BMI from 25 up to 30 kg/m²; obesity as a BMI greater than or equal to 30 kg/m², as stated above, and severe (or morbid) obesity is defined as a BMI greater than or quality to 40 kg/m².

As used herein, “sarcopenia” means a loss of skeletal muscle mass, quality, and strength. Often sarcopenia is attributed to ageing, but is also associated with HIV infection. Sarcopenia may lead to frailty, for example, in the elderly.

As used herein, “wasting syndrome” means a condition characterized by involuntary weight loss associated with chronic fever and diarrhea. In some instances, patients with wasting syndrome lose 10% of baseline body weight within one month.

As used herein, “cachexia” means a metabolic defect often associated with cancer that is characterized by progressive weight loss due to the deletion of adipose tissue and skeletal muscle.

The following abbreviations and terms have the indicated meanings throughout:
DIBAL-H=Diisobutylaluminium hydride
DIEA=N,N′-diisopropylethylamine
DMF=N,N-dimethylformamide
g=gram
h, hr, hrs=hour or hours
min=minute
mL=milliliter
NMP=N-methylpyrrolidinone
THF=tetrahydrofuran
Volume=mL/g of material based on the limiting reagent unless specified otherwise

As used in the present specification, the following words and phrases are generally intended to have the meanings as set forth below, except to the extent that the context in which they are used indicates otherwise.

As used herein, when any variable occurs more than one time in a chemical formula, its definition on each occurrence is independent of its definition at every other occurrence.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms. Alkyl groups generally are those of C₂₀ or below, such as C₁₃ or below, for example, C₆ or below. For example C₁-C₆alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. For example, C₀ alkylene indicates a covalent bond and C₁ alkylene is a methylene group. When an alkyl residue having a specific number of carbons is named, all geometric isomers having that number of carbons are intended to be encompassed; thus, for example, “butyl” is meant to include n-butyl, sec-butyl, isobutyl and tert-butyl; “propyl” includes n-propyl and isopropyl. “Lower alkyl” refers to alkyl groups having one to four carbons.

“Cycloalkyl” indicates a saturated hydrocarbon ring or fused bicyclic ring, having the specified number of carbon atoms, usually from 3 to 12 ring carbon atoms, more usually 3 to 10, or 3 to 7. Examples of cycloalkyl groups include cyclopropyl, cyclobutyl, cyclopentyl, and cyclohexyl as well as bridged and caged saturated ring groups such as norbornane. Examples of fused bicyclic rings include octahydro-1H-indene, octahydropentalene, 1,2,3,3a,4,5-hexahydropentalene, 1,2,4,5,6,7,7a-heptahydro-2H-indene, 4,5,6,7-tetrahydro-2H-indene and the like.

By “alkoxy” is meant an alkyl group attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. The alkyl group of an alkoxy group generally is of C₂₀ or below, such as C₁₃ or below, for example, C₆ or below. “Lower alkoxy” refers to alkoxy groups having one to four carbons.

By “cycloalkoxy” is meant a cycloalkyl group attached through an oxygen bridge such as, for example, cyclopropoxy, cyclobutoxy, cyclopentoxy, cyclohexoxy, cycloheptoxy, and the like. The cycloalkyl group of a cycloalkoxy group generally is of C₂₀ or below, such as C₁₃ or below, for example, C₆ or below.

“Acyl” refers to the groups (alkyl)-C(O)—; (cycloalkyl)-C(O)—; (aryl)-C(O)—; (heteroaryl)-C(O)—; and (heterocycloalkyl)-C(O)—, wherein the group is attached to the parent structure through the carbonyl functionality and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃(C═O)—.

By “alkoxycarbonyl” is meant an ester group of the formula (alkoxy)(C═O)— attached through the carbonyl carbon wherein the alkoxy group has the indicated number of carbon atoms. Thus a C₁-C₆alkoxycarbonyl group is an alkoxy group having from 1 to 6 carbon atoms attached through its oxygen to a carbonyl linker.

By “amino” is meant the group —NH₂.

The term “aminocarbonyl” refers to the group —CONR^{b}R^{c}, where
R^{b} is chosen from hydrogen, optionally substituted C₁-C₆ alkyl, optionally substituted aryl, and optionally substituted heteroaryl; and
R^{c} is chosen from hydrogen and optionally substituted C₁-C₄ alkyl; or
R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring; where each substituted group is independently substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

“Aryl” encompasses: 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene.

For example, aryl includes 5- and 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

The term “aryloxy” refers to the group —O-aryl.

In the term “arylalkyl” or “aralkyl”, aryl and alkyl are as defined herein, and the point of attachment is on the alkyl group. This term encompasses, but is not limited to, benzyl, phenethyl, phenylvinyl, phenylallyl and the like.

The term “halo” includes fluoro, chloro, bromo, and iodo, and the term “halogen” includes fluorine, chlorine, bromine, and iodine.

“Haloalkyl” indicates alkyl as defined above having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Heteroaryl” encompasses: 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring.

For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds one, those heteroatoms are not adjacent to one another. In certain embodiments, the total number of S and O atoms in the heteroaryl group is not more than two. In certain embodiments, the total number of S and O atoms in the aromatic heterocycloalkyl is not more than one. Also included within the definition of heteroaryl are oxide derivatives, for example N-oxides of nitrogen containing rings, such as pyridine-1-oxide, S-oxides of sulfur containing rings, such as >S(O) and >S(O)₂ derivatives. Examples of heteroaryl groups include, but are not limited to, systems (as numbered from the linkage position assigned priority 1), such as 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridizinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above.

In the term “heteroaralkyl,” heteroaryl and alkyl are as defined herein, and the point of attachment is on the alkyl group. This term encompasses, but is not limited to, pyridylmethyl, thienylmethyl, and (pyrrolyl)ethyl.

“Heterocycloalkyl” refers to a cycloalkyl residue in which one to four of the carbons is replaced by a heteroatom such as oxygen, nitrogen or sulfur. Also included are 4-, 5-, 6- or 7-membered non-aromatic rings containing 1-4 heteroatoms, bicyclic 8-, 9- or 10-membered non-aromatic ring systems containing 1-4 (or more) heteroatoms, or tricyclic 11- to 14-membered non-aromatic ring systems containing 1-4 (or more) heteroatoms; where the heteroatoms are selected from O, N or S. Examples include pyrrolidine, tetrahydrofuran, tetrahydro-thiophene, thiazolidine, piperidine, tetrahydro-pyran, tetrahydro-thiopyran, piperazine, morpholine, thiomorpholine and dioxane. Heterocycloalkyl also includes ring systems including unsaturated bonds, provided the number and placement of unsaturation does not render the group aromatic. Examples include imidazoline, oxazoline, tetrahydroisoquinoline, benzodioxan, benzodioxole and 3,5-dihydrobenzoxazinyl. Examples of substituted heterocycloalkyl include 4-methyl-1-piperazinyl and 4-benzyl-1-piperidinyl. Also included within the definition of heterocycloalkyl are oxide derivatives, for example N-oxides of nitrogen containing rings, such as pyridine-1-oxide, S-oxides of sulfur containing rings such as >S(O) and >S(O)₂ derivatives.

“Substituted” alkyl, cycloalkyl, aryl, heteroaryl and heterocycloalkyl refer respectively to alkyl, cycloalkyl, aryl, heteroaryl and heterocycloalkyl wherein one or more (up to about 5, for example, up to about 3) hydrogen atoms are replaced by a substituent independently selected from the group: acyl, optionally substituted alkyl (e.g., fluoroalkyl), optionally substituted alkoxy, alkylenedioxy (e.g. methylenedioxy), optionally substituted amino (e.g., alkylamino and dialkylamino), optionally substituted amidino, optionally substituted aryl (e.g., phenyl), optionally substituted aryloxy (e.g., phenoxy), optionally substituted aralkoxy (e.g., benzyloxy), carboxy (—COOH), carboalkoxy (i.e., acyloxy or —OOCR), alkoxycarbonyl or carboxyalkyl (i.e., esters or —COOR), carboxamido, aminocarbonyl, benzyloxycarbonylamino (CBZ-amino), cyano, oxo (as a substitutent for cycloalkyl, heterocycloalkyl, or heteroaryl), halogen, hydroxy, optionally substituted heteroaryl, optionally substituted heteroaralkyl, optionally substituted heteroaryloxy, optionally substituted heteroaralkoxy, nitro, sulfanyl, sulfinyl, sulfonyl, and thio.

The term “sulfanyl” includes the groups: —S-(optionally substituted alkyl), —S-(optionally substituted aryl), —S-(optionally substituted heteroaryl), and —S-(optionally substituted heterocycloalkyl). Hence, sulfanyl includes the group C₁-C₆ alkylsulfanyl.

The term “sulfinyl” includes the groups: —S(O)—H, —S(O)-(optionally substituted alkyl), —S(O)-optionally substituted aryl), —S(O)-optionally substituted heteroaryl), —S(O)-(optionally substituted heterocycloalkyl); and —S(O)-(optionally substituted amino).

The term “sulfonyl” includes the groups: —S(O₂)—H, —S(O₂)-(optionally substituted alkyl), —S(O₂)-optionally substituted aryl), —S(O₂)-optionally substituted heteroaryl), —S(O₂)-(optionally substituted heterocycloalkyl), and —S(O₂)-(optionally substituted amino).

The term “substituted,” as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (up to 5, such as up to 3) hydrogen atoms are replaced by a substituent independently chosen from:
—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —SOR^{a}, SO₂R^{a}, —SO₂NR^{b}R^{c}, and —NR^{c}SO₂R^{a}, where R^{a} is chosen from optionally substituted C₁-C₆ alkyl, optionally substituted aryl, and optionally substituted heteroaryl;
R^{b} is chosen from hydrogen, optionally substituted C₁-C₆ alkyl, optionally substituted aryl, and optionally substituted heteroaryl; and
R^{c} is chosen from hydrogen and optionally substituted C₁-C₄ alkyl; or
R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring; where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted acyl” refers to the groups (substituted alkyl)-C(O)—; (substituted cycloalkyl)-C(O)—; (substituted aryl)-C(O)—; (substituted heteroaryl)-C(O)—; and (substituted heterocycloalkyl)-C(O)—, wherein the group is attached to the parent structure through the carbonyl functionality and wherein substituted alkyl, cycloalkyl, aryl, heteroaryl, and heterocycloalkyl, refer respectively to alkyl, cycloalkyl, aryl, heteroaryl, and heterocycloalkyl wherein one or more (up to 5, such as up to 3) hydrogen atoms are replaced by a substituent independently chosen from:
—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, CONR^{b}R^{c}, —OCOR^{b}, —OCOR^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},
where R^{a} is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and
R^{c} is chosen from hydrogen and optionally substituted C₁-C₄ alkyl; or
R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring; where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). One or more carbons in the substituted acyl residue may be replaced by nitrogen, oxygen or sulfur as long as the point of attachment to the parent remains at the carbonyl.

The term “substituted alkoxy” refers to alkoxy wherein the alkyl constituent is substituted (i.e., —O-(substituted alkyl)) wherein “substituted alkyl” refers to alkyl wherein one or more (up to 5, such as up to 3) hydrogen atoms are replaced by a substituent independently chosen from:
—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, SOR^{a}, SO₂R^{a}, —SO₂NR^{b}R^{c}, and —NR^{c}SO₂R^{a}, where R^{a} is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and R^{c} is chosen from hydrogen and optionally substituted C₁-C₄ alkyl; where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

The term “substituted alkoxycarbonyl” refers to the group (substituted alkyl)-O—C(O)— wherein the group is attached to the parent structure through the carbonyl functionality and wherein substituted refers to alkyl wherein one or more (up to 5, such as up to 3) hydrogen atoms are replaced by a substituent independently chosen from:
—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a}, where R^{a} is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and
R^{c} is chosen from hydrogen and optionally substituted C₁-C₄ alkyl; or
R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring; where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted amino” refers to the group —NHR^{d} or —NR^{d}R^{d} where each R^{d} is independently chosen from: optionally substituted alkyl, optionally substituted cycloalkyl, optionally substituted acyl, optionally substituted aryl, optionally substituted heteroaryl, optionally substituted heterocycloalkyl, alkoxycarbonyl, sulfinyl and sulfonyl, wherein substituted alkyl, cycloalkyl, aryl, heterocycloalkyl, and heteroaryl refer respectively to alkyl, cycloalkyl, aryl, heterocycloalkyl, and heteroaryl wherein one or more (up to 5, such as up to 3) hydrogen atoms are replaced by a substituent independently chosen from:

—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, CO₂R^{b}, CONR^{b}R^{c}, —NR^{c}COR^{b}, —SOR^{a}, —SO₂R^{a}, —SO₂NR^{b}R^{c}, and —NR^{c}SO₂R^{a},
where R^{a} is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and
R^{c} is chosen from hydrogen and optionally substituted C₁-C₄ alkyl; or
R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring; where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl), and wherein optionally substituted acyl, alkoxycarbonyl, sulfinyl and sulfonyl are as defined herein.

Compounds of Formula I include, but are not limited to, optical isomers of compounds of Formula I, racemates, and other mixtures thereof. In addition, compounds of Formula I include Z- and E-forms (or cis- and trans-forms) of compounds with carbon-carbon double bonds. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. Where compounds of Formula I exists in various tautomeric forms, chemical entities of the present invention include all tautomeric forms of the compound.

Compounds of Formula I also include crystalline and amorphous forms of the compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

Chemical entities of the present invention include, but are not limited to compounds of Formula I and all pharmaceutically acceptable forms thereof. Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, chelates, non-covalent complexes, prodrugs, and mixtures.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH2)n-COOH where n ranges from 0 to 4, and like salts. Similarly, pharmaceutically acceptable cations include, but are not limited to sodium, potassium, calcium, aluminum, lithium, and ammonium.

In addition, if the compound of Formula I is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, prodrugs also fall within the scope of chemical entities, for example ester or amide derivatives of the compounds of Formula I. The term “prodrugs” includes any compounds that become compounds of Formula I when administered to a patient, e.g., upon metabolic processing of the prodrug. Examples of prodrugs include, but are not limited to, acetate, formate, and benzoate and like derivatives of functional groups (such as alcohol or amine groups) in the compounds of Formula I.

The term “solvate” refers to the chemical entity formed by the interaction of a solvent and a compound. Suitable solvates are pharmaceutically acceptable solvates, such as hydrates, including, for example, hemi-hydrates, monohydrates, dihydrates, trihydrates, etc.

The term “chelate” refers to the chemical entity formed by the coordination of a compound to a metal ion at two (or more) points.

The term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

The term “active agent” is used to indicate a chemical entity which has biological activity. In certain embodiments, an “active agent” is a compound having pharmaceutical utility.

The term “therapeutically effective amount” of a chemical entity of this invention means an amount effective, when administered to a human or non-human patient, to treat a disease, e.g., a therapeutically effective amount may be an amount sufficient to treat a disease or disorder responsive to myosin activation. The therapeutically effective amount may be ascertained experimentally, for example by assaying blood concentration of the chemical entity, or theoretically, by calculating bioavailability.

By “significant” is meant any detectable change that is statistically significant in a standard parametric test of statistical significance such as Student's T-test, where p<0.05.

“Patient” refers to an animal, such as a mammal, for example a human, that has been or will be the object of treatment, observation or experiment. The methods of the invention can be useful in both human therapy and veterinary applications. In some embodiments, the patient is a mammal, and in some embodiments the patient is human.

“Treatment” or “treating” means any treatment of a disease in a patient, including:
(a) preventing the disease, that is, causing the clinical symptoms of the disease not to develop;
(b) inhibiting the disease;
(c) slowing or arresting the development of clinical symptoms; and/or
(d) relieving the disease, that is, causing the regression of clinical symptoms.

As used herein, “modulation” refers to a change in one or more of diskeletal myosin, skeletal actin, skeletal tropomyosin, skeletal troponin C, skeletal troponin I, skeletal troponin T, and skeletal muscle, including fragments and isoforms thereof, as well as the skeletal sarcomere as a direct or indirect response to the presence of at least one chemical entity described herein, relative to the activity of the myosin or sarcomere in the absence of the compound. The change may be an increase in activity (potentiation) or a decrease in activity (inhibition), and may be due to the direct interaction of the compound with myosin or the sarcomere, or due to the interaction of the compound with one or more other factors that in turn effect one or more of diskeletal myosin, skeletal actin, skeletal tropomyosin, skeletal troponin C, skeletal troponin I, skeletal troponin T, and skeletal muscle, including fragments and isoforms thereof, as well as the skeletal sarcomere.

The compounds of Formula I can be named and numbered (e.g., using NamExpert™ available from Cheminnovation or the automatic naming feature of ChemDraw Ultra version 9.0 from Cambridge Soft Corporation) as described below. For example, the compound:

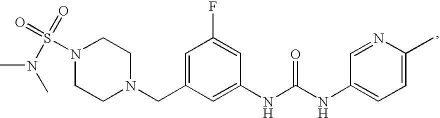

i.e., the compound according to Formula I where W, X, Y and Z are —C═, m is zero, n is one, R₁ is substituted piperazinyl, R₂ is 6-methyl-pyridin-3-yl, R₃ is hydrogen, R₄ is fluoro, R₅ is hydrogen, R₆ is hydrogen, R₇ is hydrogen, R₁₃ is hydrogen, R₁₈ is absent, and R₁₉ is absent can be named 4-(3-fluoro-5-(3-(6-methylpyridin-3-yl)ureido)benzyl)-N,N-dimethylpiperazine-1-sulfonamide.

Likewise, the compound:

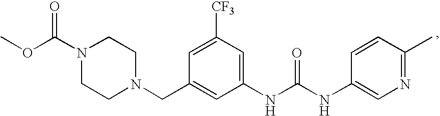

i.e., the compound according to Formula I where W, X, Y and Z are —C═, m is zero, n is one, R₁ is substituted piperazinyl, R₂ is 6-methyl-pyridin-3-yl, R₃ is hydrogen, R₄ is trifluoromethyl, R₅ is hydrogen, R₆ is hydrogen, R₇ is hydrogen, R₁₃ is hydrogen, R₁₈ is absent, and R₁₉ is absent, can be named methyl 4-(3-(3-(6-methylpyridin-3-yl)ureido)-5-(trifluoromethyl)benzyl)piperazine-1-carboxylate.

Likewise, the compound:

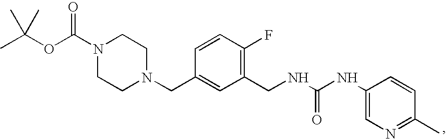

i.e., the compound according to Formula I where W, X, Y and Z are —C═, m is one, n is one, R₁ is substituted piperazinyl, R₂ is 6-methyl-pyridin-3-yl, R₃ is hydrogen, R₄ is hydrogen, R₅ is fluoro, R₆ is hydrogen, R₇ is hydrogen, R₁₃ is hydrogen, R₁₈ is hydrogen, and R₁₉ is hydrogen, can be named tert-butyl 4-(4-fluoro-3-((3-(6-methylpyridin-3-yl)ureido)methyl)benzyl)piperazine-1-carboxylate.

The chemical entities described herein can be synthesized utilizing techniques well known in the art, e.g., as illustrated below with reference to the Reaction Schemes.

Unless specified to the contrary, the reactions described herein take place at atmospheric pressure, generally within a temperature range from −10° C. to 110° C. Further, except as employed in the Examples or as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

Many of the optionally substituted starting compounds 101, 103, 201, 301 and other reactants are commercially available, e.g., from Aldrich Chemical Company (Milwaukee, Wis.) or can be readily prepared by those skilled in the art using commonly employed synthetic methodology.

Preparation of Compounds of Formula 105

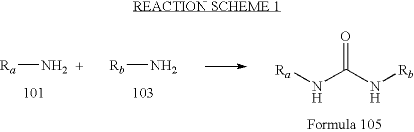

Referring to Reaction Scheme 1, a flask equipped with a magnetic stirrer, reflux condenser and thermal well, under nitrogen, is charged with phosgene or a phosgene equivalent (typically triphosgene) and a nonpolar, aprotic solvent such as dichloromethane or tetrahydrofuran. A solution of a compound of Formula 101 in a nonpolar, aprotic solvent such as dichloromethane or tetrahydrofuran is added dropwise over about 10 to 60 minutes and the solution is allowed to stir from 1 to 15 hr. A compound of Formula 103 is added portionwise, and the solution is stirred for about 10 to 60 min. A base, such as DIEA, is added dropwise for about one hour, and the solution is allowed to stir for about 1 to 15 hr. The product, a compound of Formula 105, is isolated and purified.

Preparation of Compounds of Formula 105

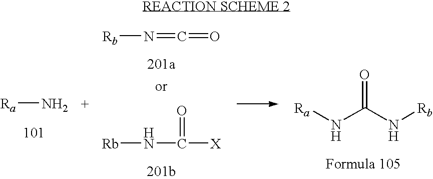

Reaction Scheme 2 illustrates an alternative synthesis of compounds of Formula 105. The isocyanate of Formula 201a can be formed and isolated independently from either corresponding amine (i.e., R_{b}—NH₂) using phosgene or a phosgene equivalent or from the corresponding carboxylic acid (i.e., R_{b}—COOH) using a Curtius or Hoffman rearrangement. Alternatively, the compound in Formula 210b wherein X is equal to a leaving group such as p-nitrophenol can be made in situ (e.g., Synthesis reference here). A mixture of compounds of Formula 101 and 201 in an aprotic solvent such as dichloromethane or tetrahydrofuran from −40° C. to 110° C. is allowed to stir from 1 to 15 hr. The product, a compound of Formula 105, is isolated and purified.

Preparation of Compounds of Formula 202

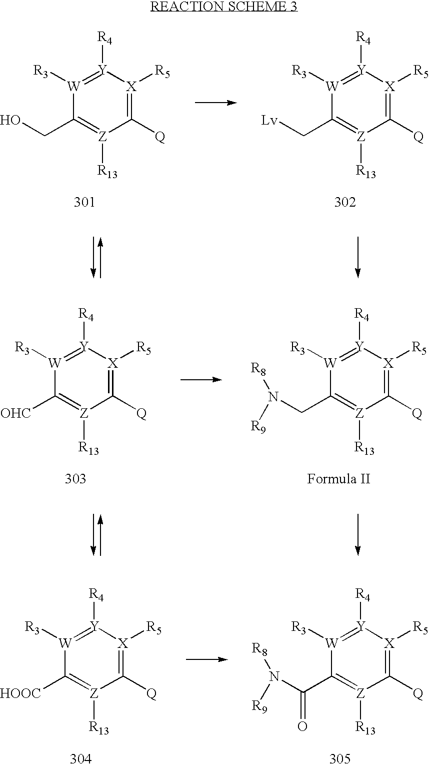

Referring to Reaction Scheme 3, the benzylic alcohol of Formula 301 is converted to a leaving group (“Lv” such as halo, mesylate or triflate) to form 302 using commonly employed synthetic methodology (for example see: “Comprehensive Organic Transformation” LaRock, Richard C., 1989, VCH publishers, Inc. p. 353-365, which is incorporated herein by reference).

A mixture of a compound of Formula 302 and amine of formula HNR₈R₉ in an aprotic solvent such as dichloromethane or DMF from −40° C. to 110° C. is allowed to stir from 1 to 15 hr. The product, a compound of Formula 202, is isolated and purified. Alternatively, the benzylic alcohol of Formula 301 is oxidized to the aldehyde of Formula 303 using commonly employed synthetic methodology (for example see: “Comprehensive Organic Transformation” LaRock, Richard C., 1989, VCH publishers, Inc. p. 604-615, which is incorporated herein by reference).

A mixture of a compound of Formula 303 and amine of formula HNR₈R₉ in a solvent such as dichloromethane with a reducing agent such as triacetoxyborohidride with or without an acid such as acetic acid from −40° C. to 110° C. is allowed to stir for between 1 to 36 hr. The product, a compound of Formula 202, is isolated and purified. Alternatively, the carboxylic acid of Formula 304 is coupled to an amine to using commonly employed synthetic methodology (for example see: “Comprehensive Organic Transformation” LaRock, Richard C., 1989, VCH publishers, Inc. pp. 972-76, which is incorporated herein by reference) to form amide 305. Amide 305 is reduced to a compound of Formula 202 using commonly employed synthetic methodology such as treating 305 with borane-dimethylsulfide in THF from −40° C. to reflux for 1 to 96 hr.

A compound of Formula 202 wherein Q is bromo, chloro, nitro, amino, or a protected amino can be conferred to a compound of Formula 101 using commonly employed synthetic methodology. Additionally Q is cyano, —CR₆R₇-bromo, —CR₆R₇-chloro, —CR₆R₇-nitro, —CR₆R₇-cyano, —CR₆R₇-amino, or a protected —CR₆R₇-amino can be conferred to a compound of Formula 101 using commonly employed synthetic methodology. For example, when Q is nitro, it may be reduced to the corresponding amine using hydrogen with a Pd/C catalyst.

Preparation of Compounds of Formula 405

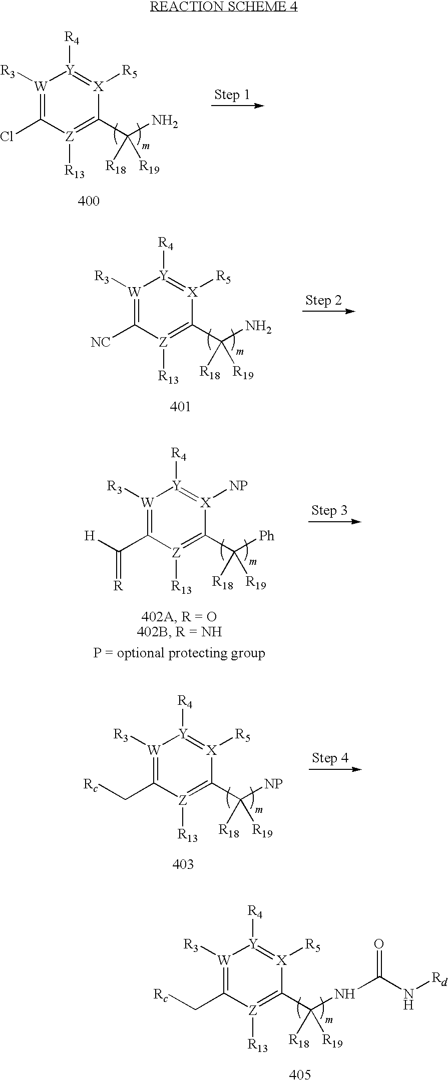

Referring to Reaction Scheme 4, Step 1, to a solution of a compound of Formula 400 in NMP is added an excess (such as about at least 2 equivalents) of sodium cyanide and an excess (such as at least 1 equivalent, for example, 1.35 equivalents) of nickel (II) bromide. Additional NMP is added, and the solution is gently warmed to about 200° C. and stirred for about 4 days. The product, a compound of Formula 401, is isolated and optionally purified.

To a ˜0° C. solution of a compound of Formula 401 in an inert solvent such as dichloromethane is added an excess (such as two or more equivalents) of a reducing agent, such as DIBAL-H (such as a 1 M solution of DIBAL-H) dropwise over ˜3.5 hours, maintaining an internal reaction temperature ≦0° C. The product, a mixture of compounds of Formula 402A and 402B, is isolated and optionally purified. Referring to Reaction Scheme 4, Step 3, to a solution of a mixture of compounds of Formula 402A and 402B in an inert solvent such as THF is added an excess (such as about 1.05 equivalents) of a compound of formula R_{c}—H wherein R_{c} is optionally substituted amino or optionally substituted heterocycloalkyl and an excess (such as about 1.5 equivalents) of a reducing agent such as triacetoxyborohydride portionwise over ˜40 min, maintaining an internal reaction temperature below about 45° C. The product, a compound of Formula 403, is isolated and optionally purified. Referring to Reaction Scheme 4, Step 4, to a solution of a compound of Formula 403 in a solvent such as acetone is added about an equivalent of a compound of formula R_{d}—NCO dropwise. The reaction is stirred for about one hour and optionally, is warmed to reflux. The product, a compound of Formula 405, is isolated and optionally purified.

A racemic mixture is optionally placed on a chromatography column and separated into (R)- and (S)-enantiomers.

The compounds described herein are optionally contacted with a pharmaceutically acceptable acid to form the corresponding acid addition salts.

Pharmaceutically acceptable acid addition salts of Formula I are optionally contacted with a base to form the corresponding free base.

In certain embodiments, the invention relates to at least one chemical entity chosen from compounds of Formula I:

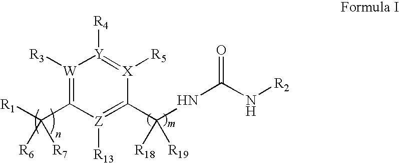

and pharmaceutically acceptable salts, chelates, non-covalent complexes, prodrugs, and mixtures thereof, wherein
W, X, Y, and Z are independently —C═ or —N═, provided that no more than two of W, X, Y, and Z are —N═;
m is zero, one, two, or three;
n is zero, one, two, or three;
R₁ is optionally substituted amino or optionally substituted heterocycloalkyl;
R₂ is optionally substituted aryl, optionally substituted aralkyl, optionally substituted cycloalkyl, optionally substituted heteroaryl, optionally substituted heteroaralkyl or optionally substituted heterocycloalkyl;
R₃ is hydrogen, halo, cyano, optionally substituted alkyl, optionally substituted heterocycloalkyl, optionally substituted alkoxy, or optionally substituted heteroaryl; or R₃ is absent when W is —N═;
R₄ is hydrogen, halo, cyano, optionally substituted alkyl, optionally substituted heterocycloalkyl, optionally substituted alkoxy, or optionally substituted heteroaryl; or R₄ is absent when Y is —N═; and
R₅ is hydrogen, halo, cyano, optionally substituted alkyl, optionally substituted heterocycloalkyl, optionally substituted alkoxy, or optionally substituted heteroaryl; or R₅ is absent when X is —N═;
R₁₃ is hydrogen, halo, cyano, hydroxyl, optionally substituted alkyl, optionally substituted heterocycloalkyl, optionally substituted alkoxy, or optionally substituted heteroaryl; or R₁₃ is absent when Z is —N═;
R₆ and R₇ are independently hydrogen, aminocarbonyl, alkoxycarbonyl, optionally substituted alkyl or optionally substituted alkoxy; or R₆ and R₇, taken together with the carbon to which they are attached, form an optionally substituted 3- to 7-membered ring which optionally incorporates one or two additional heteroatoms, chosen from N, O, and S in the ring; and
R₁₈ and R₁₉ are independently hydrogen, aminocarbonyl, alkoxycarbonyl, optionally substituted alkyl or optionally substituted alkoxy, or R₁₈ and R₁₉, taken together with the carbon to which they are attached, form an optionally substituted 3- to 7-membered ring which optionally incorporates one or two additional heteroatoms, chosen from N, O, and S in the ring; or R₁₈ and R₁₉ are absent when m is zero.

In some embodiments, W is —C═. In other embodiments, W is —N═.

In some embodiments, X is —C═. In other embodiments, X is —N═.

In some embodiments, Y is —C═. In other embodiments, Y is —N═.

In some embodiments, Z is —C═. In other embodiments, Z is —N═.

In some embodiments, none of W, X, Y, and Z are —N═, i.e., each of W, X, Y, and Z are —C═. In some embodiments, one of W, X, Y, and Z are —N═. In other embodiments, two of W, X, Y, and Z are —N═.

In some embodiments, m is zero. In other embodiments, m is one. In yet other embodiments, m is two. In other embodiments, m is three.

In some embodiments, n is zero. In other embodiments, n is one. In other embodiments, m is two. In yet other embodiments, m is three.

In some embodiments, R₁ is chosen from optionally substituted amino. In some embodiments, R₁ is —NR₈R₉ wherein R₈ is lower alkyl and R₉ is optionally substituted alkyl, optionally substituted heterocycloalkyl, optionally substituted acyl or optionally substituted sulfonyl. In some embodiments R₁ is amino.

In some embodiments, R₁ is optionally substituted heterocycloalkyl. In some embodiments, R₁ is selected from optionally substituted piperazinyl; optionally substituted 1,1-dioxo-1λ⁶-[1,2,5]thiadiazolidin-2-yl; optionally substituted 3-oxo-tetrahydro-pyrrolo[1,2-c]oxazol-6-yl, optionally substituted 2-oxo-imidazolidin-1-yl; optionally substituted morpholinyl; optionally substituted 1,1-dioxo-1λ⁶-thiomorpholin-4-yl; optionally substituted pyrrolidin-1-yl; optionally substituted piperidine-1-yl, optionally substituted azepanyl, optionally substituted 1,4-diazepanyl, optionally substituted 3-oxo-tetrahydro-1H-oxazolo[3,4-a]pyrazin-3(5H)-one, optionally substituted 5,6,7,8-tetrahydro-[1,2,4]triazolo[4,3-a]pyrazinyl, optionally substituted

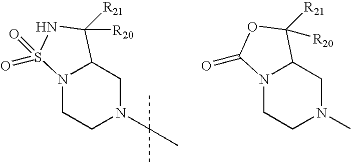

wherein R₂₀ and R₂₁ are independently hydrogen, optionally substituted alkyl, or R₂₀ and R₂₁ taken together with the carbon to which they are attached, form an optionally substituted 3- to 7-membered ring which optionally incorporates one or two additional heteroatoms, selected from N, O, and S in the ring.

In some embodiments, R₁ is substituted piperazinyl; optionally substituted piperidine-1-yl, optionally substituted pyrrolidin-1-yl, optionally substituted azepanyl or optionally substituted 1,4-diazepanyl. In some embodiments, R₁ is optionally substituted piperazinyl or optionally substituted piperidinyl.

In some embodiments, R₁ is optionally substituted piperazinyl.

In some embodiments, R₁ is optionally substituted piperidinyl.

In some embodiments, R₂ is optionally substituted aryl or optionally substituted heteroaryl. In certain embodiments, R₂ is optionally substituted phenyl, optionally substituted naphthyl, optionally substituted pyrrolyl, optionally substituted thiazolyl, optionally substituted isooxazolyl, optionally substituted pyrazolyl, optionally substituted oxazolyl, optionally substituted 1,3,4-oxadiazolyl, optionally substituted pyridinyl, optionally substituted pyrazinyl, optionally substituted pyrimidinyl and optionally substituted pyridazinyl.

In some embodiments, R₂ is chosen from pyridin-3-yl, pyridin-4-yl, pyridin-1-oxide, phenyl, pyrimidin-5-yl, and isoxazol-3-yl, wherein each of pyridin-3-yl, pyridin-4-yl, pyridin-1-oxide, phenyl, pyrimidin-5-yl, and isoxazol-3-yl is optionally substituted with optionally substituted lower alkyl, lower alkoxy, halo (such as fluoro or chloro), cyano or acyl. In certain embodiments, R₂ is pyridin-3-yl, which is optionally substituted with lower alkyl, cyano, or acetyl or with lower alkyl substituted with one or more halo groups; R₂ is pyridin-4-yl which is optionally substituted with lower alkyl; phenyl which is optionally substituted with halo; optionally substituted pyrimidin-5-yl; or optionally substituted isoxazol-3-yl. In certain embodiments, R₂ is pyridin-3-yl; 6-methyl-pyridin-3-yl; 6-cyano-pyridin-3-yl; 6-acetyl-pyridin-3-yl; 6-trifluoromethyl-pyridin-3-yl; pyridin-4-yl; 2-methyl-pyridin-4-yl; phenyl; 4-fluorophenyl; 4-chlorophenyl; or 5-methyl-isoxazol-3-yl.

In some embodiments, R₃ is chosen from hydrogen, cyano, optionally substituted alkyl, halo, and optionally substituted alkoxy. In some embodiments, R₃ is chosen from hydrogen, cyano, optionally substituted lower alkyl, halo, and optionally substituted lower alkoxy. In some embodiments, R₃ is methyl, ethyl, trifluoromethyl, difluoromethyl, trifluoromethoxy, difluoromethoxy, chloro, fluoro, or hydrogen.

In some embodiments, R₄ is chosen from hydrogen, pyridinyl, halo, and optionally substituted alkyl. In some embodiments, R₄ is chosen from hydrogen, pyridinyl, halo, and optionally substituted lower alkyl. In some embodiments, R₄ is hydrogen, fluoro, methyl, trifluoromethyl, or pyridinyl.

In some embodiments, R₅ is chosen from hydrogen, pyridinyl, halo, optionally substituted alkyl, and optionally substituted alkoxy. In some embodiments, R₅ is hydrogen, methyl, chloro, fluoro, difluoromethyl, trifluoromethyl, trifluoromethoxy, difluoromethoxy, or methoxy.

In some embodiments, R₆ and R₇ are independently hydrogen, aminocarbonyl, alkoxycarbonyl, optionally substituted alkyl or optionally substituted alkoxy. In some embodiments, R₆ and R₇, taken together with the carbon to which they are attached, form an optionally substituted 3- to 7-membered ring which optionally incorporates one or two additional heteroatoms, selected from N, O, and S in the ring.

In some embodiments, only one of R₆ and R₇ is hydrogen or R₆ and R₇ are both hydrogen. In some embodiments, one or both of R₆ and R₇ are optionally substituted alkyl. In some embodiments, one or both of R₆ and R₇ are methyl.

In certain embodiments, R₆ and R₇ are independently hydrogen or methyl.

In certain embodiments, n is one and R₆ and R₇ are independently hydrogen or methyl.

In certain embodiments, n is one and R₆ is methyl and R₇ is hydrogen. In certain embodiments, n is two and each R₆ and R₇ is hydrogen. In certain embodiments, n is three and each R₆ and R₇ is hydrogen.

In some embodiments, R₈ is methyl or ethyl.

In some embodiments, R₉ is —(CO)OR₁₀ wherein R₁₀ is hydrogen or lower alkyl (such as methyl or ethyl). In certain embodiments, R₁₀ is hydrogen, methyl or ethyl.

In some embodiments, R₉ is —(SO₂)—R₁₇ wherein R₁₇ is lower alkyl (such as methyl or ethyl) or —NR₁₁R₁₂ wherein R₁₁ and R₁₂ are independently hydrogen or lower alkyl (such as methyl or ethyl).

In some embodiments, R₉ is alkyl optionally substituted with optionally substituted amino. In some embodiments, R₉ is methyl or ethyl.

In some embodiments, R₉ is optionally substituted heterocycloalkyl.

In some embodiments, R₁₃ is chosen from hydrogen, halo, cyano, and hydroxyl. In some embodiments, R₁₃ is fluoro.

In some embodiments, R₁₃ is hydrogen, cyano, lower alkyl (such as methyl or ethyl), hydroxyl, or halo. In certain embodiments, R₁₃ is hydrogen or fluoro.

In some embodiments, R₁₈ and R₁₉ are independently hydrogen, aminocarbonyl, alkoxycarbonyl, optionally substituted alkyl or optionally substituted alkoxy. In other embodiments, R₁₈ and R₁₉, taken together with the carbon to which they are attached, form an optionally substituted 3- to 7-membered ring which optionally incorporates one or two additional heteroatoms, selected from N, O, and S in the ring.

In some embodiments, only one of R₁₈ and R₁₉ is hydrogen or R₁₈ and R₁₉ are both hydrogen. In some embodiments, one or both of R₁₈ and R₁₈ are optionally substituted alkyl. In some embodiments, one or both of R₁₈ and R₁₉ are methyl.

In some embodiments, R₁₈ and R₁₉ are independently hydrogen or methyl. In certain embodiments, R₁₈ and R₁₉ are independently hydrogen or methyl. In certain other embodiments, m is zero and R₁₈ and R₁₉ are absent. In certain embodiments, m is one and R₁₈ and R₁₉ are independently hydrogen or methyl. In certain embodiments, m is one and R₁₈ is methyl and R₁₉ is hydrogen. In certain embodiments, m is two and each R₁₈ and R₁₉ is hydrogen. In certain embodiments, m is three and each R₁₈ and R₁₉ is hydrogen.

In some embodiments, R₃, R₄, R₅, and R₁₃ are hydrogen. In certain embodiments, one of R₃, R₄, R₅, and R₁₃ is not hydrogen.

In some embodiments, one of R₃, R₄, R₅, and R₁₃ is halo, optionally substituted lower alkyl, or cyano and the others are hydrogen. In certain embodiments one of R₃, R₄, R₅, and R₁₃ is halo, methyl or cyano and the others are hydrogen. In certain embodiments two of R₃, R₄, R₅, and R₁₃ are halo or cyano and the others are hydrogen.

In some embodiments, one of R₃, R₄, R₅, and R₁₃ is fluoro and the others are hydrogen. In certain embodiments, one of R₃, R₄, R₅, and R₁₃ is cyano and the others are hydrogen. In certain embodiments, two of R₃, R₄, R₅, and R₁₃ are not hydrogen. In certain embodiments, two of R₃, R₄, R₅, and R₁₃ are halo and the others are hydrogen. In some embodiments, two of R₃, R₄, R₅, and R₁₃ are fluoro and the others are hydrogen.

In some embodiments, the methods employ a chemical entity of Formula I chosen from a chemical entity of Formula II:

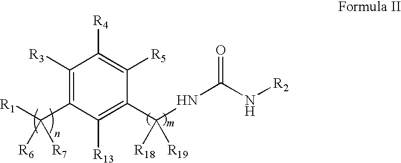

wherein R₁, R₂, R₃, R₄, R₅, R₆, R₇, R₁₃, R₁₈, R₁₉, m, and n are as described for compounds of Formula I.

In certain embodiments, the methods employ a chemical entity of Formula I chosen from a chemical entity of Formula III:

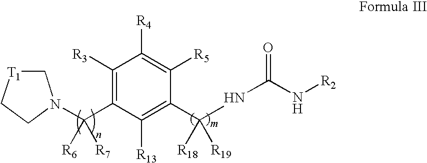

wherein R₂, R₃, R₄, R₅, R₆, R₇, R₁₃, R₁₈, R₁₉, m, and n are as described for compounds of Formula I and wherein:

T₁ is chosen from —CHR₁₄—, —NR₁₅CHR₁₄—, —CHR₁₄NR₁₅—, and —CHR₁₄CHR₁₄—; and
each R₁₄ and R₁₅ is independently chosen from hydrogen, optionally substituted alkyl, optionally substituted acyl, carboxy, optionally substituted lower alkoxycarbonyl, optionally substituted aminocarbonyl, optionally substituted alkoxy, optionally substituted cycloalkoxy, optionally substituted sulfonyl, optionally substituted amino, optionally substituted cycloalkyl, and optionally substituted heterocycloalkyl.

In some embodiments, T₁ is —NR₁₅CHR₁₄—, i.e., R₁ is a piperazinyl ring substituted with R₁₄ and R₁₅. In certain embodiments, T₁ is —CHR₁₄CHR₁₄—.

In some embodiments, R₁₄ and R₁₅ are independently selected from hydrogen, methyl, carboxy, methoxycarbonyl, ethoxycarbonyl, propoxycarbonyl, isopropoxycarbonyl, tert-butoxycarbonyl, benzyloxy carbonyl, N,N-dimethylcarbamoyl, acetyl, propionyl, isobutyryl, propoxy, methoxy, cyclohexylmethyloxy, methylsulfonyl, ethylsulfonyl, n-propylsulfonyl, isopropylsulfonyl, azetidin-1-ylsulfonyl, dimethylamino sulfonyl, methanesulfonamido, N-methyl-methanesulfonamido, ethanesulfonamido, N-methyl-ethanesulfonamido, N-methoxycarbonyl-N-methylamino, N-ethoxycarbonyl-N-methylamino, N-isopropoxycarbonyl-N-methylamino, N-tert-butoxycarbonyl-N-methylamino, acetamido, N-methylacetamido, N-methylpropionamido, N-methylisobutyramido, amino, methylamino, dimethylamino, N-methyl-(dimethylamino sulfonyl)amino, and piperidin-1-yl.

In some embodiments, R₁₄ is chosen from hydrogen, methyl, and methoxymethyl.

In some embodiments, R₁₅ is chosen from optionally substituted acyl, optionally substituted lower alkoxycarbonyl, and optionally substituted sulfonyl. In certain embodiments, R₁₅ is chosen from lower alkoxycarbonyl, lower alkylsulfonyl, and optionally substituted aminosulfonyl.

In certain embodiments the methods employ a chemical entity of Formula I chosen from a chemical entity of Formula IV:

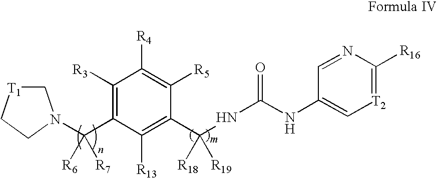

wherein T₁, R₃, R₄, R₅, R₆, R₇, R₁₃, R₁₈, R₁₉, m, and n are as described for compounds of Formula III and wherein T₂ is —C═ or —N═; and
R₁₆ is selected from hydrogen, halo, cyano, optionally substituted acyl, optionally substituted alkyl, and optionally substituted alkoxy.

In some embodiments, T₂ is —C═.

In some embodiments, T₂ is —N═.

In some embodiments, R₁₆ is selected from hydrogen, methyl, fluoro, cyano, methoxy, and acetyl. In certain embodiments, R₁₆ is hydrogen or methyl.

In certain embodiments, the compound of Formula I is:
4-(3-fluoro-5-(3-(6-methylpyridin-3-yl)ureido)benzyl)-N,N-dimethylpiperazine-1-sulfonamide;
(E)-N′-cyano-4-(3-fluoro-5-(3-(6-methylpyridin-3-yl)ureido)benzyl)-N,N-dimethylpiperazine-1-carboximidamide;
N-(1-(3-fluoro-5-(3-(6-methylpyridin-3-yl)ureido)benzyl)piperidin-4-yl)-N-methylethanesulfonamide;
N-(1-(3-fluoro-5-(3-(6-methylpyridin-3-yl)ureido)benzyl)piperidin-4-yl)-N-methyl(dimethylamino)sulfonamide;
N-(1-(3-fluoro-5-(3-(pyridin-3-yl)ureido)benzyl)piperidin-4-yl)-N-methyl(dimethylamino)sulfonamide;
N-(1-(3-fluoro-5-(3-(4-fluorophenyl)ureido)benzyl)piperidin-4-yl)-N-methyl(dimethylamino)sulfonamide;
1-(3-fluoro-5-((3-oxo-tetrahydro-1H-oxazolo[3,4-a]pyrazin-7(3H)-yl)methyl)phenyl)-3-(6-methylpyridin-3-yl)urea;
methyl 4-(1-(3-fluoro-5-(3-(6-methylpyridin-3-yl)ureido)phenyl)ethyl)piperazine-1-carboxylate;
ethyl 4-(1-(3-fluoro-5-(3-(6-methylpyridin-3-yl)ureido)phenyl)ethyl)piperazine-1-carboxylate;
methyl 4-(2-fluoro-3-(3-(6-methylpyridin-3-yl)ureido)benzyl)piperazine-1-carboxylate;
methyl 4-(3-(3-fluoro-5-(3-(6-methylpyridin-3-yl)ureido)phenyl)propyl)piperazine-1-carboxylate;
1-(3-(3-(4-(ethylsulfonyl)piperazin-1-yl)propyl)-5-fluorophenyl)-3-(6-methylpyridin-3-yl)urea;
4-(3-(3-fluoro-5-(3′-(6-methylpyridin-3-yl)ureido)phenyl)propyl)-N,N-dimethylpiperazine-1-sulfonamide;
methyl 4-(3-(3-(6-methylpyridin-3-yl)ureido)-5-(trifluoromethyl)benzyl)piperazine-1-carboxylate;
methyl 4-(3-(3-(6-methylpyridin-3-yl)ureido)-4-(trifluoromethyl)benzyl)piperazine-1-carboxylate;
(R)-ethyl 4-(1-(3-fluoro-5-(3-(6-methylpyridin-3-yl)ureido)phenyl)ethyl)piperazine-1-carboxylate;
(S)-tert-butyl 4-(1-(3-(3-(6-methylpyridin-3-yl)ureido)phenyl)ethyl)piperazine-1-carboxylate;
(S)-methyl 4-(1-(2-fluoro-3-(3-(6-methylpyridin-3-yl)ureido)phenyl)ethyl)piperazine-1-carboxylate;
(S)-1-(3-(1-(4-acetylpiperazin-1-yl)ethyl)-2-fluorophenyl)-3-(6-methylpyridin-3-yl)urea;
methyl 4-(2,5-difluoro-3-(3-(6-methylpyridin-3-yl)ureido)benzyl)piperazine-1-carboxylate;
methyl 4-(3-(2-fluoro-3-(3-(6-methylpyridin-3-yl)ureido)phenyl)propyl)piperazine-1-carboxylate;
methyl 4-(2-hydroxy-3-(3-(6-methylpyridin-3-yl)ureido)benzyl)piperazine-1-carboxylate;
ethyl 4-(2-hydroxy-3-(3-(6-methylpyridin-3-yl)ureido)benzyl)piperazine-1-carboxylate;
1-(3-(3-(4-acetylpiperazin-1-yl)propyl)-2-fluorophenyl)-3-(6-methylpyridin-3-yl)urea;
ethyl 4-(3-(2-fluoro-3-(3-(6-methylpyridin-3-yl)ureido)phenyl)propyl)piperazine-1-carboxylate;
tert-butyl 4-(2-fluoro-3-(3-(6-methylpyridin-3-yl)ureido)benzyl)piperazine-1-carboxylate;
ethyl 4-(2-fluoro-3-(3-(6-methylpyridin-3-yl)ureido)benzyl)piperazine-1-carboxylate;
1-(3-((4-acetylpiperazin-1-yl)methyl)-2-fluorophenyl)-3-(6-methylpyridin-3-yl)urea;
4-(2-fluoro-3-(3-(6-methylpyridin-3-yl)ureido)benzyl)-N,N-dimethylpiperazine-sulfonamide;
4-(2-fluoro-3-(3-(6-methylpyridin-3-yl)ureido)benzyl)-N,N-dimethylpiperazine-1-carboxamide;
1-(3-((4-(ethylsulfonyl)piperazin-1-yl)methyl)-2-fluorophenyl)-3-(6-methylpyridin-3-yl)urea;
1-(2-fluoro-3-((4-(methylsulfonyl)piperazin-1-yl)methyl)phenyl)-3-(6-methylpyridin-3-yl)urea;
(2S,6R)-methyl 4-(2-fluoro-3-(3-(6-methylpyridin-3-yl)ureido)benzyl)-2,6-dimethylpiperazine-1-carboxylate;
methyl 4-(2-fluoro-3-(3-(4-fluorophenyl)ureido)benzyl)piperazine-1-carboxylate;
methyl 4-(3-(3-(6-cyanopyridin-3-yl)ureido)-2-fluorobenzyl)piperazine-1-carboxylate;
methyl 4-(3-(3-(6-acetylpyridin-3-yl)ureido)-2-fluorobenzyl)piperazine-1-carboxylate;
methyl 4-(2-fluoro-3-(3-(6-(trifluoromethyl)pyridin-3-yl)ureido)benzyl)piperazine-1-carboxylate;
methyl 4-(2-fluoro-3-(3-pyridin-4-yl)ureido)benzyl)piperazine-1-carboxylate;
1-(3-((4-(azetidin-1-ylsulfonyl)piperazin-1-yl)methyl)-2-fluorophenyl)-3-(6-methylpyridin-3-yl)urea;
(3S,5R)-tert-butyl 4-(2-fluoro-3-(3-(6-methylpyridin-3-yl)ureido)benzyl)-3,5-dimethylpiperazine-1-carboxylate;
1-(3-(((2S,6R)-4-(ethylsulfonyl)-2,6-dimethylpiperazin-1-yl)methyl)-2-fluorophenyl)-3-(6-methylpyridin-3-yl)urea;
(3S,5R)-4-(2-fluoro-3-(3-(6-methylpyridin-3-yl)ureido)benzyl)-N,N,3,5-tetramethylpiperazine-1-sulfonamide;
tert-butyl 4-(4-fluoro-3-((3-(6-methylpyridin-3-yl)ureido)methyl)benzyl)piperazine-1-carboxylate;
methyl 4-(4-fluoro-3-((3-(6-methylpyridin-3-yl)ureido)methyl)benzyl)piperazine-1-carboxylate;
ethyl 4-(4-fluoro-3-((3-(6-methylpyridin-3-yl)ureido)methyl)benzyl)piperazine-1-carboxylate;
1-(5-((4-acetylpiperazin-1-yl)methyl)-2-fluorobenzyl)-3-(6-methylpyridin-3-yl)urea;
1-(5-((4-(ethylsulfonyl)piperazin-1-yl)methyl)-2-fluorobenzyl)-3-(6-methylpyridin-3-yl)urea;
4-(4-fluoro-3-((3-(6-methylpyridin-3-yl)ureido)methyl)benzyl)-N,N-dimethylpiperazine-1-sulfonamide;
4-(2-chloro-5-((3-(6-methylpyridin-3-yl)ureido)methyl)benzyl)-N,N-dimethylpiperazine-1-sulfonamide;
1-(4-chloro-3-((4-cyanopiperazin-1-yl)methyl)benzyl)-3-(6-methylpyridin-3-yl)urea;
N,N-dimethyl-4-(2-methyl-5-((3-(6-methylpyridin-3-yl)ureido)methyl)benzyl)piperazine-1-sulfonamide;
methyl 4-(4-(difluoromethoxy)-3-((3-(6-methylpyridin-3-)ureido)methyl)benzyl)piperazine-1-carboxylate;
ethyl 4-(4-(difluoromethoxy)-3-((3-(6-methylpyridin-3-yl)ureido)methyl)benzyl)piperazine-1-carboxylate;
ethyl 4-(3-((3-(4-cyanophenyl)ureido)methyl)-4-fluorobenzyl)piperazine-1-carboxylate;
1-(2-fluoro-5-((4-(methylsulfonyl)piperazin-1-yl)methyl)benzyl)-3-(6-methylpyridin-3-yl)urea;
isopropyl 4-(4-fluoro-3-((3-(6-methylpyridin-3-yl)ureido)methyl)benzyl)piperazine-1-carboxylate;
1-(2-fluoro-5-((4-(isopropylsulfonyl)piperazin-1-yl)methyl)benzyl)-3-(6-methylpyridin-3-yl)urea;
1-(2-fluoro-5-((4-(3-methylbutanoyl)piperazin-1-yl)methyl)benzyl)-3-(6-methylpyridin-3-yl)urea;
1-(2-fluoro-5-((4-(propylsulfonyl)piperazin-1-yl)methyl)benzyl)-3-(6-methylpyridin-3-yl)urea;
1-(2-fluoro-5-((4-pivaloylpiperazin-1-yl)methyl)benzyl)-3-(6-methylpyridin-3-yl)urea;
methyl 4-(2-(difluoromethoxy)-5-((3-(6-methylpyridin-3-yl)ureido)methyl)benzyl)piperazine-1-carboxylate;
ethyl 4-(2-(difluoromethoxy)-5-((3-(6-methylpyridin-3-yl)ureido)methyl)benzyl)piperazine-1-carboxylate;
1-(4-(difluoromethoxy)-3-((4-(ethylsulfonyl)piperazin-1-yl)methyl)benzyl)-3-(6-methylpyridin-3-yl)urea;
1-(4-(difluoromethoxy)-3-((4-(methylsulfonyl)piperazin-1-yl)methyl)benzyl)-3-(6-methylpyridin-3-yl)urea;
ethyl 4-(4-fluoro-3-((3-(3-methylisoxazol-5-yl)ureido)methyl)benzyl)piperazine-1-carboxylate;
ethyl 4-(3-((3-(6-acetylpyridin-3-yl)ureido)methyl)-4-fluorobenzyl)piperazine-1-carboxylate;
ethyl 4-(4-methyl-3-((3-(6-methylpyridin-3-yl)ureido)methyl)benzyl)piperazine-1-carboxylate;
isopropyl 4-(4-methyl-3-((3-(6-methylpyridin-3-yl)ureido)methyl)benzyl)piperazine-1-carboxylate;
1-(5-((4-(ethylsulfonyl)piperazin-1-yl)methyl)-2-methylbenzyl)-3-(6-methylpyridin-3-yl)urea;
1-(5-((4-acetylpiperazin-1-yl)methyl)-2-methylbenzyl)-3-(6-methylpyridin-3-yl)urea;
1-(5-((4-(isopropylsulfonyl)piperazin-1-yl)methyl)-2-methylbenzyl)-3-(6-methylpyridin-3-yl)urea;
1-(5-((4-isobutyrylpiperazin-1-yl)methyl)-2-methylbenzyl)-3-(6-methylpyridin-3-yl)urea;
ethyl 4-(2,4-difluoro-3-((3-(6-methylpyridin-3-yl)ureido)methyl)benzyl)piperazine-1-carboxylate;
1-(6-cyanopyridin-3-yl)-3-(5-((4-(ethylsulfonyl)piperazin-1-yl)methyl)-2-fluorobenzyl)urea;
1-(6-acetylpyridin-3-yl)-3-(5-((4-(ethylsulfonyl)piperazin-1-yl)methyl)-2-fluorobenzyl)urea;
1-(5-((4-(ethylsulfonyl)piperazin-1-yl)methyl)-2-fluorobenzyl)-3-(6-methoxypyridin-3-yl)urea;
tert-butyl 4-(4-chloro-3-((3-(6-methylpyridin-3-yl)ureido)methyl)benzyl)piperazine-1-carboxylate;
1-(2-fluoro-3-((4-(methylsulfonyl)piperazin-1-yl)methyl)benzyl)-3-(pyridin-4-yl)urea;
1-(3-((4-(ethylsulfonyl)piperazin-1-yl)methyl)-2-fluorophenyl)-3-(pyridin-4-yl)urea;
(R)-tert-butyl 4-(2-fluoro-3-(3-(6-methylpyridin-3-yl)ureido)benzyl)-3-methylpiperazine-1-carboxylate;
(R)-methyl 4-(2-fluoro-3-(3-(6-methylpyridin-3-yl)ureido)benzyl)-3-methylpiperazine-1-carboxylate;
(R)-ethyl 4-(2-fluoro-3-(3-(6-methylpyridin-3-yl)ureido)benzyl)-3-methylpiperazine-1-carboxylate;
(R)-1-(3-((4-(ethylsulfonyl)-2-methylpiperazin-1-yl)methyl)-2-fluorophenyl)-3-(6-methylpyridin-3-yl)urea;
1-(2-fluoro-3-((4-(methylsulfonyl)piperazin-1-yl)methyl)phenyl)-3-(2-methylpyridin-4-yl)urea;
1-(3-((4-(ethylsulfonyl)piperazin-1-yl)methyl)-2-fluorophenyl)-3-(2-methylpyridin-4-yl)urea;
methyl 4-(2-fluoro-3-(3-(2-methylpyridin-4-yl)ureido)benzyl)piperazine-1-carboxylate;
1-(2-fluoro-3-((4-(isopropylsulfonyl)piperazin-1-yl)methyl)phenyl)-3-(6-methylpyridin-3-yl)urea;
1-(2-fluoro-3-((4-(propylsulfonyl)piperazin-1-yl)methyl)phenyl)-3-(6-methylpyridin-3-yl)urea;
1-(3-((4-(cyclopropylsulfonyl)piperazin-1-yl)methyl)-2-fluorophenyl)-3-(6-methylpyridin-3-yl)urea;
(R)-4-(2-fluoro-3-(3-(6-methylpyridin-3-yl)ureido)benzyl)-N,N,3-trimethylpiperazine-1-sulfonamide;
(R)-1-(2-fluoro-3-((2-methyl-4-(methylsulfonyl)piperazin-1-yl)methyl)phenyl)-3-(6-methylpyridin-3-yl)urea;
(R)-1-(3-((4-acetyl-2-methylpiperazin-1-yl)methyl)-2-fluorophenyl)-3-(6-methylpyridin-3-yl)urea;
(S)-4-(2-fluoro-3-(3-(6-methylpyridin-3-yl)ureido)benzyl)-N,N,3-trimethylpiperazine-1-sulfonamide;
1-(2-chloro-5-((4-(ethylsulfonyl)piperazin-1-yl)methyl)phenyl)-3-(6-methylpyridin-3-yl)urea;
1-(3-((4-(azetidin-1-ylsulfonyl)piperazin-1-yl)methyl)-2-fluorophenyl)-3-(pyridin-4-yl)urea;
(R)-1-(3-((4-(azetidin-1-ylsulfonyl)-2-methylpiperazin-1-yl)methyl)-2-fluorophenyl)-3-(6-methylpyridin-3-yl)urea;
(S)-1-(3-((4-(azetidin-1-ylsulfonyl)-2-methylpiperazin-1-yl)methyl)-2-fluorophenyl)-3-(6-methylpyridin-3-yl)urea; and
1-(2-fluoro-3-((4-(isopropylsulfonyl)piperazin-1-yl)methyl)phenyl)-3-(2-methylpyridin-4-yl)urea.

The chemical entities described herein modulate one or more of diskeletal myosin, skeletal actin, skeletal tropomyosin, skeletal troponin C, skeletal troponin I, skeletal troponin T, and skeletal muscle, including fragments and isoforms thereof, as well as the skeletal sarcomere, and are useful to bind to, inhibit and/or potentiate the activity thereof. As used in this context, “modulate” means either increasing or decreasing myosin activity, whereas “potentiate” means to increase activity and “inhibit” means to decrease activity.

The chemical entities, pharmaceutical compositions and methods of the invention are used to treat obesity, sarcopenia, wasting syndrome, frailty, muscle spasm, cachexia, i neuromuscular diseases (e.g., amyotrophic lateral sclerosis, spinal muscular atrophy, familial or acquired myopathies or muscular dystrophies), post-surgical and post-traumatic muscle weakness, and other conditions in a mammal.

Methods to identify the chemical entities as binding to a protein or as a modulator of the binding characteristics or biological activity of a protein are described in, for example, U.S. Pat. No. 6,410,254 and U.S. patent application Ser. No. 10/987,165, each hereby incorporated by reference.

The chemical entities described herein are administered at a therapeutically effective dosage, e.g., a dosage sufficient to provide treatment for the disease states previously described. While human dosage levels have yet to be optimized for the chemical entities described herein, generally, a daily dose ranges from about 0.05 to 100 mg/kg of body weight; in certain embodiments, from about 0.10 to 10.0 mg/kg of body weight, and in certain embodiments, from about 0.15 to 1.0 mg/kg of body weight. Thus, for administration to a 70 kg person, in certain embodiments, the dosage range would be about from 3.5 to 7000 mg per day; in certain embodiments, about from 7.0 to 700.0 mg per day, and in certain embodiments, about from 10.0 to 100.0 mg per day. The amount of the chemical entity administered will, of course, be dependent on the subject and disease state being treated, the severity of the affliction, the manner and schedule of administration and the judgment of the prescribing physician; for example, a likely dose range for oral administration would be from about 70 to 700 mg per day, whereas for intravenous administration a likely dose range would be from about 70 to 700 mg per day depending on compound pharmacokinetics.

Administration of the chemical entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarily, vaginally, rectally, or intraocularly. Oral and parenteral administration are customary in treating the indications that are the subject of the present invention.

Pharmaceutically acceptable compositions include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In addition, the chemical entities described herein can be co-administered with, and the pharmaceutical compositions can include, other medicinal agents, pharmaceutical agents, adjuvants, and the like. Suitable medicinal and pharmaceutical agents include modulators of one or more of diskeletal myosin, skeletal actin, skeletal tropomyosin, skeletal troponin C, skeletal troponin I, skeletal troponin T, and skeletal muscle, including fragments and isoforms thereof, and the skeletal sarcomere and other suitable therapeutic agents useful in the treatment of the aforementioned disorders, as well as the agents described in U.S. Patent Application No. 2005/0197367.

Suitable additional medicinal and pharmaceutical agents include, for example: orlistat, sibramine, diethylpropion, phentermine, benzaphetamine, phendimetrazine, estrogen, estradiol, levonorgestrel, norethindrone acetate, estradiol valerate, ethinyl estradiol, norgestimate, conjugated estrogens, esterified estrogens, medroxyprogesterone acetate, testosterone, insulin-derived growth factor, human growth hormone, riluzole, cannabidiol, prednisone, albuterol, non-steroidal anti-inflammatory drugs, and botulinum toxin.

Other suitable medicinal and pharmaceutical agents include TRH, diethylstilbesterol, theophylline, enkephalins, E series prostaglandins, compounds disclosed in U.S. Pat. No. 3,239,345 (e.g., zeranol), compounds disclosed in U.S. Pat. No. 4,036,979 (e.g., sulbenox), peptides disclosed in U.S. Pat. No. 4,411,890 growth hormone secretagogues such as GHRP-6, GHRP-1 (disclosed in U.S. Pat. No. 4,411,890 and publications WO 89/07110 and WO 89/07111), GHRP-2 (disclosed in WO 93/04081), NN703 (Novo Nordisk), LY444711 (Lilly), MK-677 (Merck), CP424391 (Pfizer) and B-HT920, growth hormone releasing factor and its analogs, growth hormone and its analogs and somatomedins including IGF-1 and IGF-2, alpha-adrenergic agonists, such as clonidine or serotonin 5-HT_{D} agonists, such as sumatriptan, agents which inhibit somatostatin or its release, such as physostigmine, pyridostigmine, parathyroid hormone, PTH(1-34), and bisphosphonates, such as MK-217 (alendronate).

Still other suitable medicinal and pharmaceutical agents include estrogen, testosterone, selective estrogen receptor modulators, such as tamoxifen or raloxifene, other androgen receptor modulators, such as those disclosed in Edwards, J. P. et. al., Bio. Med. Chem. Let., 9, 1003-1008 (1999) and Hamann, L. G. et. al., J. Med. Chem., 42, 210-212 (1999), and progesterone receptor agonists (“PRA”), such as levonorgestrel, medroxyprogesterone acetate (MPA).

Still other suitable medicinal and pharmaceutical agents include aP2 inhibitors, such as those disclosed in U.S. Ser. No. 09/519,079 filed Mar. 6, 2000, PPAR gamma antagonists, PPAR delta agonists, beta 3 adrenergic agonists, such as AJ9677 (Takeda/Dainippon), L750355 (Merck), or CP331648 (Pfizer), other beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), a thyroid receptor beta drug, such as a thyroid receptor ligand as disclosed in WO 97/21993, WO 99/00353, and GB98/284425, and anorectic agents, such as dexamphetamine, phentermine, phenylpropanolamine or mazindol.

Still other suitable medicinal and pharmaceutical agents include HIV and AIDS therapies, such as indinavir sulfate, saquinavir, saquinavir mesylate, ritonavir, lamivudine, zidovudine, lamivudine/zidovudine combinations, zalcitabine, didanosine, stavudine, and megestrol acetate.

Still other suitable medicinal and pharmaceutical agents include antiresorptive agents, hormone replacement therapies, vitamin D analogues, elemental calcium and calcium supplements, cathepsin K inhibitors, MMP inhibitors, vitronectin receptor antagonists, Src SH.sub.2 antagonists, vacular —H⁺-ATPase inhibitors, ipriflavone, fluoride, Tibo lone, pro stanoids, 17-beta hydroxysteroid dehydrogenase inhibitors and Src kinase inhibitors.

The above other therapeutic agents, when employed in combination with the chemical entities described herein, may be used, for example, in those amounts indicated in the Physicians' Desk Reference (PDR) or as otherwise determined by one of ordinary skill in the art.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

The following examples serve to more fully describe the manner of using the above-described invention. It is understood that these examples in no way serve to limit the true scope of this invention, but rather are presented for illustrative purposes. All references cited herein are incorporated by reference in their entirety.

### EXAMPLE 1

### Step 1

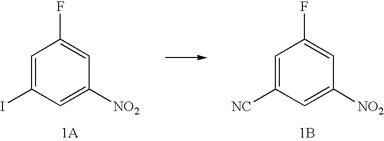

To a solution of 1.0 eq 1A in dry DMF (0.37 M) was added Zn(CN)₂ (0.92 eq) and Pd(PPh₃)₄ (0.058 eq). The reaction mixture was purged with nitrogen and heated to 80° C. overnight. An additional 0.023 eq of Pd(PPh₃)₄ was then added and the reaction was heated for another 6 hrs. The reaction mixture was then cooled to RT, diluted with 15 volumes of EtOAc (based on 1A) and the organic layer was washed 3 times with water and once with brine. The organic layer was dried over sodium sulfate, filtered and concentrated. Purification by chromatography over silica gel using 10% Et₂O/hexane as the eluant provided 1B as a solid (90%).

### EXAMPLE 1

### Step 2

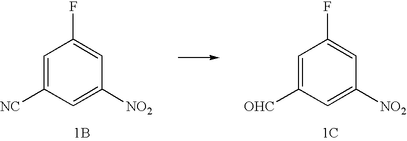

To solution of 1.0 eq 1B in dry Et₂O (0.06 M) at 0° C. was added dropwise a solution of diisobutyllithiumaluminum hydride (1.1 eq, 1.0 M in hexanes) by syringe. The resulting solution was kept at 0° C. overnight. The reaction mixture was added to a mixture of ice and glacial acetic acid. The reaction mixture was then diluted with ethyl acetate, and the aqueous layer was extracted with ethyl acetate two additional times. The combined organic layers were washed twice with saturated sodium bicarbonate, and once with brine. The organic layers were then dried over sodium sulfate, filtered and concentrated in vacuo. Purification over silica gel using 10% EtOAc/hexanes as the eluant afforded a yellow solid (100%) as an 80:20 mixture of 1C:1B.

### EXAMPLE 1

### Step 3

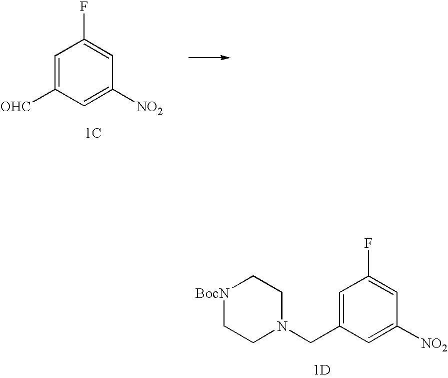

To cooled (0° C.) slurry of an 80:20 mixture of 1C:1B (1.0 eq) and boc-piperazine (about 2 eq) in a mixture of HOAc and DCM (4.8 M boc-piperazine in 1:1.4 v/v HOAc/DCM) was added sodium triacetoxyborohydride as a solid over about 5 minutes. The reaction was allowed to warm to RT and stirred for two hours. The reaction mixture was quenched with saturated sodium bicarbonate and diluted with ethyl acetate. The layers were separated and the aqueous layer was washed three times with ethyl acetate. The organic layers were combined and washed with brine, dried over sodium sulfate, and concentrated in vacuo. Purification by chromatography over silica gel using 50% ethyl acetate/hexanes as the eluant provided 1D (67.7%) as a yellow oil.

### EXAMPLE 1

### Step 4

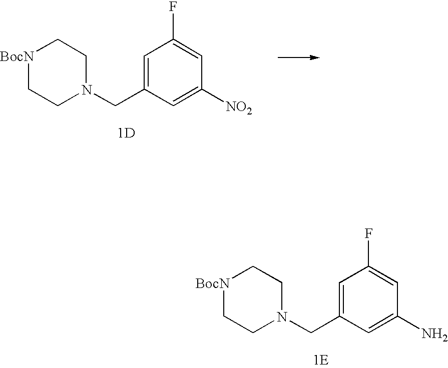

A mixture of 1.0 eq of 1D, and a catalytic amount of 10% Pd/C (approximately 10 wt/wt %) in MeOH (about 0.6 M 1D in MeOH) was stirred over an atmosphere of 50 psi H₂ for 45 min. After replacement of the H₂ atmosphere with N₂, the reaction mixture was filtered through diatomaceous earth and the diatomaceous earth washed with MeOH. Concentration of the MeOH resulted in the isolation of 1E.

### EXAMPLE 1

### Step 5

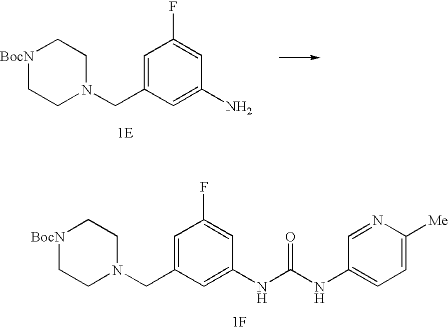

To a solution of aniline 1E (1.0 eq) in dry DCM (about 0.1 M 1E in DCM) at RT under N₂ atmosphere was added the 2-methyl-5-isocyanatopyridine (slight excess, about 1.2 eq) by syringe. The mixture was stirred for 1 hour. To the reaction mixture was added sequentially saturated aqueous sodium bicarbonate and ethyl acetate. The layers were separated and the organic layer was washed twice with sat. NaHCO₃ and once with brine. The organic layer was dried over sodium sulfate, filtered and concentrated in vacuo. Purification by chromatography over silica gel using 5% methanol/DCM as the eluant provided 1F.

### EXAMPLE 1

### Steps 6 and 7

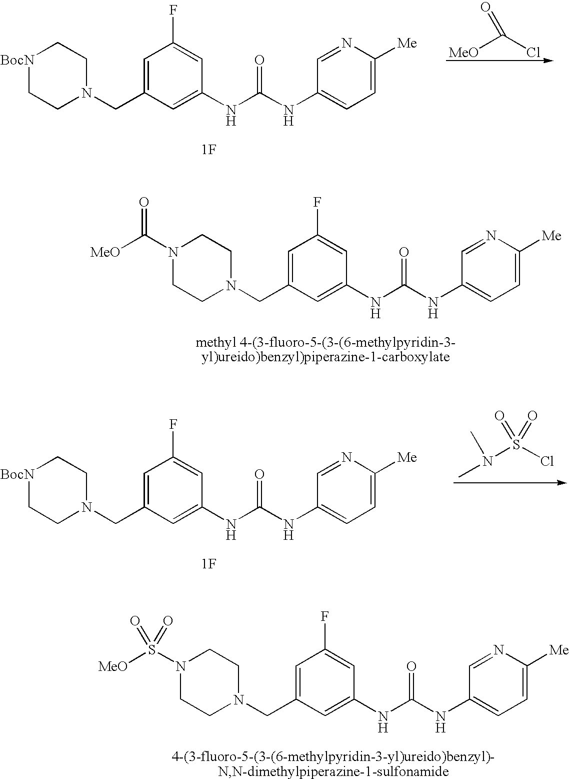

To a solution of 1.0 eq of 1F in CH₂Cl₂ (about 0.14 M 1F in DCM) was added approximately 200 eq of trifluoroacetic acid (TFA). The reaction mixture was stirred for 30 min and concentrated. The resultant residue was dissolved in EtOAc (about 1.6 times the volume of the reaction mixture) and washed sequentially with 3N NaOH (2 times) and brine. The organic layer was dried (NaSO4) and concentrated to provided the desired free base that was used without further purification.

To a solution of the free base above (1.0 eq) and DIPEA (1.2 eq) in dry THF (about 0.2 M free base in THF) was added methyl chloroformate (1.1 eq) by syringe and the resultant mixture stirred for 1 h. To the mixture was added aqueous sodium bicarbonate followed by ethyl acetate. The organic layer was separated and washed twice with aqueous sodium bicarbonate and once with brine. The combined aqueous layers were extracted once with ethyl acetate. The combined organic layers were dried over sodium sulfate, filtered and concentrated in vacuo. Purification by chromatography over silica gel using 5% MeOH/DCM as the eluant provided methyl 4-(3-fluoro-5-(3-(6-methylpyridin-3-yl)ureido)benzyl)-piperazine-1-carboxylate. MS 402 (M+H).

To a solution of the free base above (1.0 eq) and DIPEA (1.2 eq) in dry THF (about 0.2 M free base in THF) was added dimethylsulfamoyl chloride (1.1 eq) by syringe. After a few hours, the reaction was complete. The mixture was quenched with aqueous sodium bicarbonate, diluted with ethyl acetate, and washed twice with bicarb and once with brine. The combined aqueous layers were extracted once with ethyl acetate, and the combined organic layers were dried over sodium sulfate, filtered and concentrated in vacuo. Purification by chromatography over silica gel using 5% MeOH/DCM as the eluant provided 4-(3-fluoro-5-(3-(6-methylpyridin-3-yl)ureido)benzyl)-N,N-dimethylpiperazine-1-sulfonamide. MS 451 (M+H).

### EXAMPLE 2

### Step 1

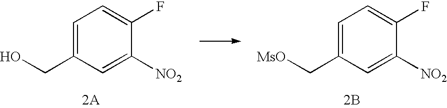

To 1.0 eq of (4-fluoro-3-nitro-phenyl)-methanol (2A) in THF (about 1 M 2A in THF) and (about 1.1 eq) of pyridine was added approximately 1.1 eq of methanesulfonyl chloride. The mixture was stirred overnight at room temperature then concentrated. The residue was purified using by flash chromatography over silica with 10%-50% EtOAc/hexanes as the eluant to yield of methanesulfonic acid 4-fluoro-3-nitro-benzyl ester (2B) (57%).

### EXAMPLE 2

### Step 2

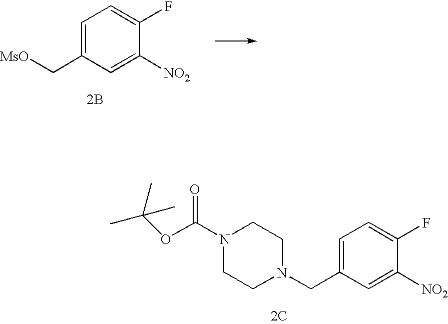

To 1.0 eq of methanesulfonic acid 4-fluoro-3-nitro-benzyl ester (2B) in DMF (about 0.6 M 2B in DMF) was added about 1.05 eq of TEA and about 1.0 eq of t-butyl piperazine-1-carboxylate. The mixture was stirred for 30 min at room temperature, diluted with EtOAc, washed with NH₄Cl solution, dried (Na₂SO₄) and evaporated. Purification by flash chromatography over silica with 50% EtOAc/hexanes as the eluant afforded 4-(4-fluoro-3-nitro-benzyl)-piperazine-1-carboxylic acid tert-butyl ester (2C).

### EXAMPLE 2

### Step 3

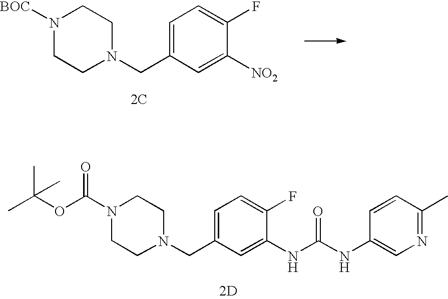

4-(4-Fluoro-3-nitro-benzyl)-piperazine-1-carboxylic acid tert-butyl ester (2C, 1.0 eq) in methanol (about 0.2 M 2C in MeOH) was treated with catalytic Pd(OH)₂/C under hydrogen at 60 psi overnight. The mixture was filtered through diatomatious earth and concentrated to an oil. This oil was dissolved in THF and treated with approximately 1.05 eq of 6-methylpyridine-3-isocyanate. After stirring at 50° C. for 30 min the mixture was concentrated. The residue was purified by reversed phase HPLC to yield 4-{4-fluoro-3-[3-(6-methyl-pyridin-3-yl)-ureido]-benzyl}-piperazine-1-carboxylic acid tert-butyl ester (2D).

### EXAMPLE 2

### Steps 4 and 5

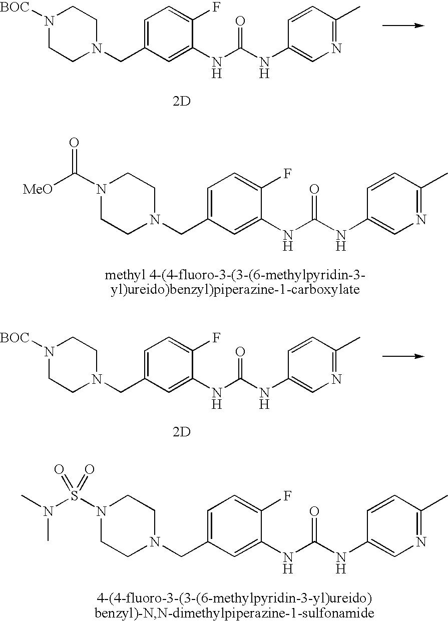

To 1.0 eq of 4-{4-fluoro-3-[3-(6-methyl-pyridin-3-yl)-ureido]-benzyl}-piperazine-1-carboxylic acid tert-butyl ester (2D) in MeOH (about 0.1 M 2D in MeOH) was added 2 volumes of HCl in dioxane (4 N) and the reaction mixture stirred at 50° C. for 15 min and evaporated to a solid. The solid was combined with DCM and treated with approximately 5 eq of TEA and split into 3 equal portions of reaction mixture A. One portion of the reaction mixture A was treated with 1.2 eq of methyl carbonyl chloride and stirred overnight. The resultant mixture was concentrated and purified by reversed phase HPLC to afford 4-{4-fluoro-3-[3-(6-methyl-pyridin-3-yl)-ureido]-benzyl}-piperazine-1-carboxylic acid methyl ester. MS 402 (M+H). A second portion of the reaction mixture A was treated with 1.2 eq of dimethylsulfamoyl chloride and stirred overnight. The resultant mixture was concentrated and purified by reversed phase HPLC to afford 4-{4-fluoro-3-[3-(6-methyl-pyridin-3-yl)-ureido]-benzyl}-piperazine-1-sulfonic acid dimethylamide. MS 451 (M+H).

### EXAMPLE 3

### Step 1

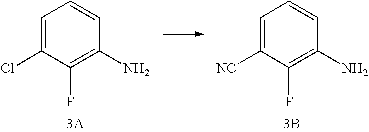

A round bottom flask was charged with 1 eq of 3-chloro-2-fluoroaniline (3A), 1-methyl-2-pyrrolidinone (about 1.5 M 3A in NMP), 2.2 eq of sodium cyanide, and 1.35 eq of nickel(II) bromide at RT under N₂. The concentration was halved by the introduction of additional NMP under N₂ and the solution was gently warmed to 200±5° C. and stirred for 4 days under N₂. The reaction mixture was allowed to cool to room temperature. The reaction mixture was diluted with 30 volumes of tert-butyl methyl ether (MTBE) and filtered through celite. The celite pad was then rinsed with 10 volumes of MTBE. The organics were washed with 40 volumes of brine, 2×40 volumes of water and 40 volumes of brine. The combined organics were dried over sodium sulfate and concentrated to afford a brown solid, which was dried under vacuum (˜30 in Hg) at 40° C. for 8 hours to afford the compound of Formula 3B (71% yield).

### EXAMPLE 3

### Step 2

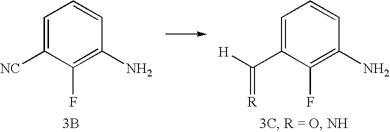

A solution of 3B in dichloromethane (about 1.5 M 3B in DCM) at RT under nitrogen mixture was cooled to ˜0° C., and 2.0 eq of 1M diisobutyllithiumaluminum hydride (DIBAlH) in DCM was added dropwise over ˜3.5 hours, maintaining an internal reaction temperature ≦0° C. Upon completion of the DiBAlH addition, the reaction mixture was added dropwise with vigorous stirring to a cooled solution (˜0° C.) of 40 volumes of 15% Rochelle salt and 10 volumes of DCM, maintaining an internal reaction temperature below 10° C. The flask was rinsed with 10 volumes of DCM and the mixture was allowed to warm to room temperature and stirred for 4 hours. The layers were separated, and the aqueous layers were back extracted with 20 volumes of DCM. The combined organic layers were washed with 20 volumes of water. The organic layer was dried over sodium sulfate and concentrated to afford a brown foam, which was dried under vacuum (˜30 in Hg) at RT to afford 3C (92% yield).

### EXAMPLE 3

### Step 3

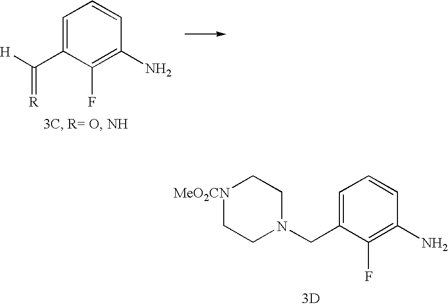

### Steps 3A/B

A solution 1 eq of 3C, tetrahydrofuran (about 1.4 M 3C in THF) and 1.05 eq of methyl piperazine-1-carboxylate and was allowed to stir at ambient temperature for 3 hours. To the reaction mixture was added 1.5 eq of sodium triacetoxyborohydride portionwise over ˜40 min, maintaining an internal reaction temperature below 45° C. The reaction mixture was stirred overnight at room temperature. To the reaction mixture was added 5 volumes of water dropwise, over 1 hour, maintaining an internal reaction temperature below 30° C. Ethyl acetate (EtOAc, 5 volumes) was then added, and the layers were separated. The aqueous layers were back extracted with 5 volumes of EtOAc. The combined organic layers were washed with saturated sodium bicarbonate and solid sodium bicarbonate was added as needed to bring the pH to 8 (pHydrion papers). The layers were separated, and the organic layer was washed with 5 volumes of brine. The organic layer was dried over sodium sulfate and activated carbon was added in the drying step. The organics were filtered through celite and the celite pad was rinsed 4 times with EtOAc. The organics were concentrated and dried overnight on the rotavap (˜30 in Hg at RT) to afford an amber-brown oil.

### Step 3C

All calculations are based on the amount of 3C (R═O).

To 3 volumes of methanol (based on 3C, R═O)under N₂ over an ice/brine/acetone bath was added 3 eq of acetyl chloride dropwise over 3 hours, maintaining an internal reaction temperature below 0° C. The solution was then stirred for an additional 1 hour below 0° C. A solution of 1.0 eq of unpurified 3D (from Steps 3A/3B above) in MeOH (about 3.6 M based on 3C, R═O) was added dropwise over 30 min, maintaining an internal reaction temperature below 15° C. The reaction was allowed to warm to room temperature overnight. The solids were filtered the next day and rinsed with 2×0.5 volumes of MeOH, 5 volumes of 1:1 tert-butyl methyl ether (MTBE):MeOH, and 5 volumes of MTBE.

The solids were then taken up in 5 volumes of EtOAc and saturated sodium bicarbonate and solid sodium bicarbonate were added as needed to bring the pH of the aqueous layer to 8 (pHydrion papers). The layers were separated, and the aqueous layer was extracted with 5 volumes of EtOAc. The combined organic layers were washed with 5 volumes of brine, dried over sodium sulfate, and concentrated to afford a pale orange solid which was dried under vacuum (˜30 in Hg) at ˜40° C. to afford 3D (50% yield).

### EXAMPLE 3

### Step 4

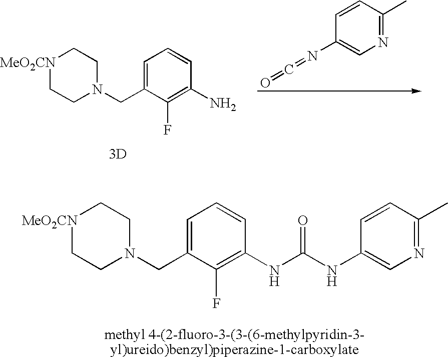

To a solution of 3D in acetone (about 2.7 M 3D in acetone) was added 1.0 eq of 5-isocyanato-2-methylpyridine dropwise over 9 min. A voluminous precipitate formed during the addition, and the reaction was stirred for one hour. The reaction mixture was warmed to reflux for 2 hours and cooled to RT for 2.5 hour. The reaction was then warmed to reflux for 1 hr and cooled to RT overnight. The reaction was filtered and rinsed with 1 volume of acetone, then three times with 2 volumes of ethyl acetate. The solids were dried under vacuum (˜30 in Hg) at 60° C. overnight to afford a white powder (86% yield) of methyl 4-(2-fluoro-3-(3-(6-methylpyridin-3-yl)ureido)benzyl)piperazine-1-carboxylate. The material was reworked as follows:

Methyl 4-(2-fluoro-3-(3-(6-methylpyridin-3-yl)ureido)benzyl)piperazine-1-carboxylate from above was dissolved in acetone (about 0.2 M) under N₂. The reaction was then warmed to reflux for 2.5 hr and cooled to RT overnight. The reaction was filtered and rinsed with 1 volume of acetone, then three times with 2 volumes of ethyl acetate. The solids were dried under vacuum (˜30 in Hg) at 60° C. overnight to afford methyl 4-(2-fluoro-3-(3-(6-methylpyridin-3-yl)ureido)benzyl)piperazine-1-carboxylate as a white powder (79% yield). The material was reworked as follows:

Methyl 4-(2-fluoro-3-(3-(6-methylpyridin-3-yl)ureido)benzyl)piper-azine-1-carboxylate from above was dissolved in acetone (about 0.2 M) under N₂. The reaction was then warmed to reflux and cooled to RT overnight. The reaction was filtered and rinsed with 1 volume of acetone, then three more times with 2 volumes of ethyl acetate. The solids were dried under vacuum (˜30 in Hg) at 60° C. overnight to afford methyl 4-(2-fluoro-3-(3-(6-methylpyridin-3-yl)ureido)benzyl)piperazine-1-carboxylate as a white powder (73% yield). MS 402 (M+H).

### EXAMPLE 4

### Step 1

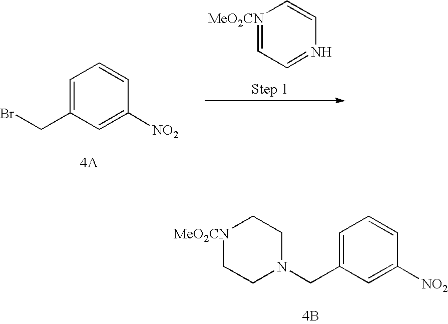

A 3-neck round bottom flask was purged with nitrogen for at least ten minutes. The flask was charged with 1.0 eq of 4A, CH₂Cl₂ (about 1.2 M 4A in DCM), and about 1.1 eq of DIPEA. The flask was then cooled to 10±5° C. While the flask was cooling, 1.2 eq of methyl piperazine-1-carboxylate was taken up in CH₂Cl₂ (about 5.3 M). The material did not go into solution, so an additional 0.05 eq of DIPEA in DCM (about 0.3 M) was added. The material did not go into solution, and the suspension was then added dropwise over 50 min, maintaining an internal reaction temperature ≦30° C. The cooling bath was removed and the reaction mixture was warmed to reflux. The reaction mixture was maintained at reflux for 19 hours. An additional 0.05 eq methyl piperazine-1-carboxylate was added, and the reaction was refluxed for another 2.5 hours. The reaction was cooled to RT and washed with 5 volumes of water. The water layer was back-extracted with 5 volumes of CH₂Cl₂. The combined organic layers were washed with 5 volumes of 10% AcOH/water. The organic layer was then washed with 5 volumes of saturated sodium bicarbonate and 5 volumes of brine. The organic layer was dried over sodium sulfate, filtered and concentrated via rotavap at 30±5° C. to a residue. MTBE was charged to the rotavap flask at 20±5° C. and the flask was rotated until a solution had been achieved. Hexane was charged into the flask and the solution stirred for 2.5 hours at 20±5° C. The solids were filtered and rinsed with hexanes. The solids were dried at ≦40° C. under maximum vacuum until constant mass was achieved (˜22 hours) to afford 4B as a pale yellow solid (66% yield).

### EXAMPLE 4

### Step 2

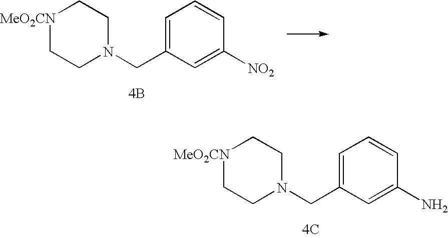

A high-pressure reactor was charged with a slurry of 25 wt % of Pt/C relative to 4B in 8 volumes of THF (relative to Pt/C) followed by a slurry of 1.5 eq K₂CO₃, in THF (about 0.67 M), then a solution of 1.0 eq of 4B in THF (about 0.47 M). The reactor jacket was set to 10° C., and the reactor was charged with 50 psi H₂ while maintaining an internal reaction temperature ≦30° C. The reaction was stirred for 9 hours, 45 min then stirred for another 3.5 hours. The reaction was filtered. The reaction flask and filters were rinsed with 9 volumes of MeOH (relative to 4B) and concentrated via rotavap at ≦50° C. The residue was dissolved in 4 volumes of EtOAc and washed with 4 volumes of water. The water layer was back-extracted with 4 volumes of EtOAc. The combined organics were washed with 4 volumes of brine, dried over sodium sulfate, filtered and concentrated via rotavap at ≦50° C. to afford a residue. Once the solvent had stopped coming off the rotovap, the residue was charged with 2 volumes of MTBE and the solution was concentrated via rotavap at ≦50° C. to afford a residue. Once the solvent had stopped coming off the rotovap, the material was kept on the rotovap under maximum vacuum for 15 hours. MTBE (2 volumes) was then charged to triturate the material and the flask rotated for 2 hours. The solids were filtered and rinsed with 0.5 volumes of MTBE. The solids were dried at ≦50° C. under maximum vacuum until constant mass was achieved (˜22 hours) to afford 4C as a pale yellow solid (87% yield).

### EXAMPLE 4

### Step 3

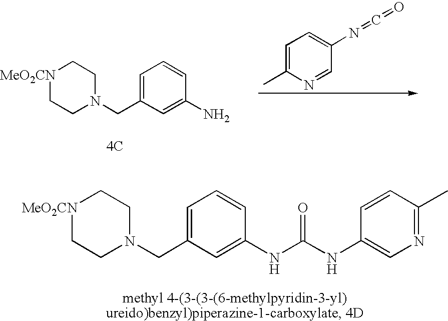

A 3-neck round bottom flask was purged with nitrogen for at least ten minutes. The flask was then charged with 1.0 eq 4C in acetone (about 0.56 M). The flask was warmed at 27° C. to form a solution. About 1 eq 5-isocyanato-2-pyridine was added dropwise over 68 min, controlling the addition rate to keep the internal temperature ≦45° C. After the addition, the reaction mixture was maintained ≦45° C. for approximately 5 hours. The reaction was then warmed to a gentle reflux for 35 min then cooled back to room temperature overnight (15 hrs). The solids were filtered and rinsed with 0.45 volumes of acetone and 1.7 volumes of EtOAc. The solids were dried in a vacuum oven ≦50° C. to afford 4D, methyl 4-(3-(3-(6-methylpyridin-3-yl)ureido)benzyl)piperazine-1-carboxylate (89% yield). MS 384 (M+H).

### EXAMPLE 5

### Step 1

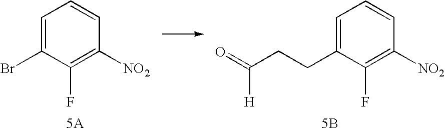

To a mixture of 1.0 eq 2-fluoro-3-bromo-nitrobenzene (5A), 1.0 eq tetrabutylammonium chloride, 1.5 eq NaHCO₃, and 2.0 eq allyl alcohol in DMF (about 1M allyl alcohol in DMF) under N₂ atmosphere was added 0.4 eq PdCl₂. The reaction mixture was warmed to 60° C. and stirred under N₂ for 16 h. The temperature was raised to 70° C. and the reaction mixture was stirred an additional 4 h. Additional aliquots of 1 eq allyl alcohol and 0.1 eq PdCl₂ were added and the reaction mixture was stirred under N₂ for 6 h. The reaction mixture was cooled to room temperature and diluted with EtOAc. The mixture was washed sequentially with water, 1N HCl, and brine. The organic layer was dried and concentrated to a residue. Purification over silica gel using 10% EtOAc/Hexane to 60% EtOAc/Hexane as the gradient eluant afforded 5B.

### EXAMPLE 5

### Step 2

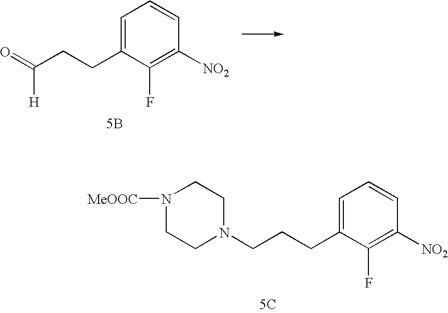

To a solution of 1.0 eq 5B in CH₂Cl₂ (about 0.04 M) under N₂ atmosphere was added 1.3 eq methyl piperazine-1-carboxylate HCl salt followed by 1.2 eq sodium triacetoxyborohydride. The reaction mixture was stirred at RT overnight. An additional 0.5 eq of methyl piperazine-1-carboxylate HCl salt followed by 2 eq of sodium triacetoxyborohydride was added to the reaction mixture and the mixture was stirred at RT for 4 h. The reaction mixture was diluted with CH₂Cl₂ and washed sequentially with water and brine. The organic layer was dried and concentrated to a residue. Purification over silica gel using 2:1 EtOAc/Hexane as the eluant afforded 5C.

### EXAMPLE 5

### Step 3

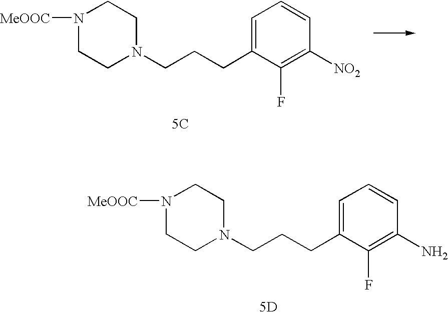

A mixture of 1 eq 5C, and 50 wt eq of 10% Pd/C in MeOH (0.06 M 5C in MeOH) was stirred over an atmosphere of 30 psi H₂ for 2 h. After replacement of the H₂ atmosphere with N₂, the reaction mixture was filtered through diatomaceous earth and the diatomaceous earth washed with MeOH. Concentration of the MeOH resulted in the isolation of 5D in nearly quantitative yield.

### EXAMPLE 5

### Step 4

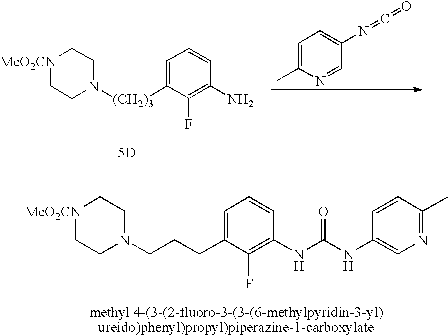

To a solution of 1 eq SD in CH₂Cl₂ (about 0.1 M) under N₂ atmosphere at RT was added 1 eq 5-isocyanato-2-pyridine and the resultant mixture was stirred at RT for 12 h. The reaction mixture was diluted with CH₂Cl₂ and washed sequentially with water and brine. The organic layer was dried and concentrated to a residue. Purification by preparative reverse phase HLPC (C-18 column) using 10% CH₃CN/water to 100% CH₃CN as the gradient eluant afforded methyl 4-(3-(2-fluoro-3-(3-(6-methylpyridin-3-yl)ureido)phenyl)propyl)piperazine-1-carboxylate. MS 430 (M+H).

### EXAMPLE 6

### Steps 1 and 2

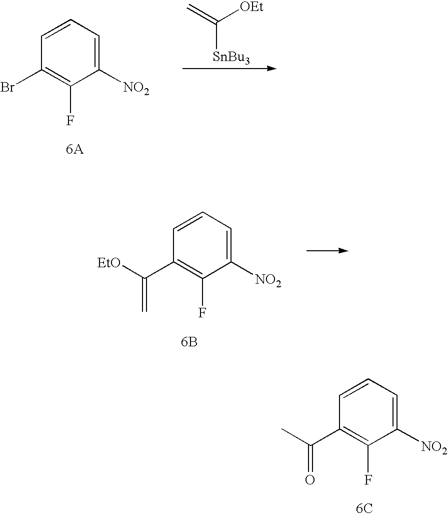

PdCl₂(PPh₃)₂ (0.05 eq) was added to a mixture of 1.0 eq of 6A, 1.0 eq of tributyl(1-ethoxyvinyl)-tin in dioxane (about 0.4 M) under N₂. The mixture was heated at 95° C. for 4 hours under N₂. A mixture of 1:1 v/v EtOAc/(1M KF) solution was added to the reaction mixture and the mixture was stirred for 1 hour. The precipitate was filtered off. The organic layer was dried and concentrated to give 6B that was used without further purification.

To a mixture of 6B in THF (0.8 M relative to 6A) was added about 2.3 volumes of 2N HCl and the mixture was stirred at RT for 1 h. Saturated NaHCO₃ was added to the reaction mixture. The reaction mixture was concentrated to remove THF and to the resultant mixture was added a volume of ether about 3 times that of the volume of the reaction mixture. The organic layer was dried and concentrated to a residue. The residue was purified over silica gel to obtain 6C (87% in 2 steps).

### EXAMPLE 6

### Step 3

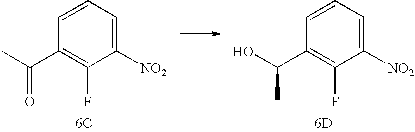

To a mixture of 0.1 to 0.15 eq of (S)-1-methyl-3,3-diphenyl-hexahydropyrrolo[1,2-c][1,3,2]oxazaborole in toluene (1-1.5 M) and toluene (a volume about 10 times that of the oxazaborole in toluene) under N₂ at 20° C. was added 1.05 eq of Et₂NPh-BH₃. To this reaction mixture was added dropwise 1.0 eq 6C in toluene (about 0.4 M) over 1.5 hours. The reaction mixture was then stirred for additional 1 hour at RT. To the reaction mixture was added about 1.9 volumes of MeOH, followed by about 3.4 volumes of 1N HCl. The mixture was stirred for 20 min. To the reaction mixture was added about 7.8 volumes of ether and about 7.8 volumes of brine. The organic layer was separated, dried and concentrated to a residue. The residue was purified by chromatography over silica gel to afford 6D (79%).

### EXAMPLE 6

### Step 4

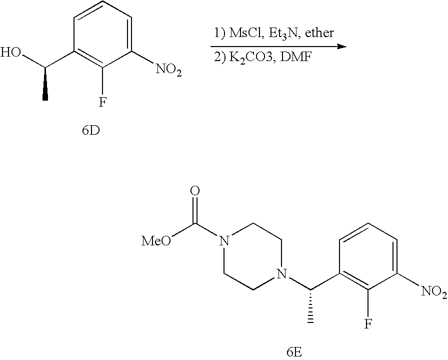

To 1.0 eq 6D in ether (about 0.55 M) and 1.2 eq Et₃N was added about 1.1 eq methanesulfonyl chloride dropwise at 0° C. The mixture was stirred at RT for 30 min. The reaction mixture was filtered and concentrated to a residue. The residue was dissolved into about 5.9 volumes of DMF and 1.2 eq methyl piperazine-1-carboxylate HCl salt and 4 eq of K₂CO₃ were added. The reaction mixture was heated at 50° C. for 16 hours. The reaction mixture was cooled to RT and about 29 volumes of EtOAc and 29 volumes sat. NH₄Cl were added. The organic layer was separated, dried, and concentrated. The resultant residue was purified by chromatography over silica gel to give 6E.

### EXAMPLE 6

### Step 5

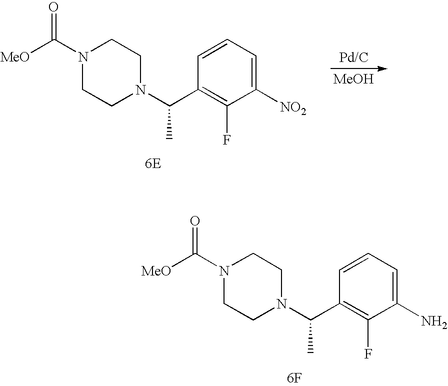

A mixture of 1 eq 6E, and 10 wt eq of 10% Pd/C in MeOH was stirred over an atmosphere of 45 psi H₂ for 0.5 h. After replacement of the H₂ atmosphere with N₂, the reaction mixture was filtered through diatomaceous earth and the diatomaceous earth washed with MeOH. Concentration of the MeOH resulted in the isolation of 6F.

### EXAMPLE 6

### Step 6

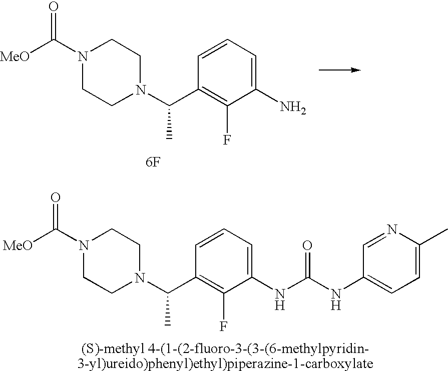

To a solution of 1.0 eq 6F in CH₂Cl₂ (at about 0.3 M) under N₂ atmosphere at RT was added 1.0 eq of 5-isocyanato-2-methylpyridine and the resultant mixture was stirred at RT for 0.5 h. The reaction mixture was concentrated to a residue. Purification by reverse phase HLPC(C-18 column) afforded (S)-methyl-4-(1-(2-fluoro-3-(3-(6-methylpyridin-3-yl)ureido)phenyl)ethyl)-piperazine1-carboxylate as a white solid. MS 416 (M+H).

### EXAMPLE 7

### Step 1

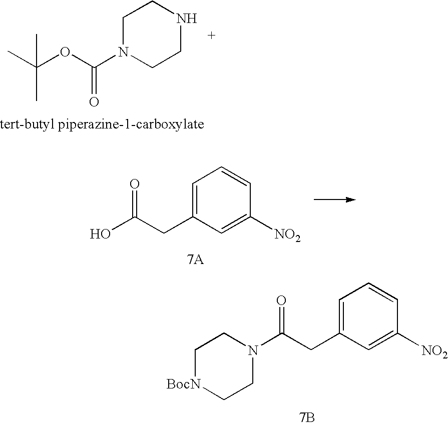

An oven-dried, round-bottom flask was charged with tert-butyl piperazine-1-carboxylate (1.1 eq), 3-nitrophenylacetic acid (7A, 1.0 eq), EDC (1.2 eq), and HOBT (1.2 eq). The flask was flushed with nitrogen, and N,N-dimethylformamide (about 0.5 M 7A in DMF) and triethylamine (2.0 eq) were added by syringe. The resulting reaction mixture was stirred overnight at room temperature. The reaction mixture was then diluted with EtOAc, and washed 4 times with H₂O, twice with 1 N aq. KHSO₄, once with saturated NaHCO₃, and once with brine. The organic layer was dried over Na₂SO₄, filtered and concentrated in vacuo. Tert-butyl 4-(2-(3-nitrophenyl)acetyl)piperazine-1-carboxylate (7B) was isolated as a solid (80%) and used without further purification.

### EXAMPLE 7

### Step 2

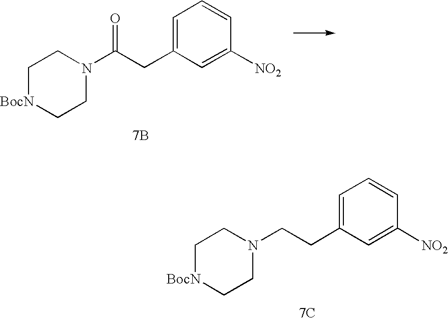

To a solution of tert-butyl 4-(2-(3-nitrophenyl)acetyl)piperazine-1-carboxylate (7B, 1.0 eq) in THF (about 0.5 M 7B in THF)) was added borane-THF (2.0 eq) by syringe. The resulting reaction mixture was heated to reflux for 2 h. The reaction mixture was cooled under an ice/water bath and 10% aq. HOAc was added, slowly. The mixture was concentrated in vacuo, and the residue was dissolved in EtOAc. The organic layer was partitioned with water, and the aqueous layer was made basic (pH˜9) by the addition of 50% NaOH. The organic layer was then washed twice with saturated aq. NaHCO₃ and once with brine. The organic layer dried over Na₂SO₄, filtered and concentrated in vacuo. The resulting tert-butyl 4-(3-nitrophenethyl)piperazine-1-carboxylate (7C, quant.) was used without further purification.

### EXAMPLE 7

### Step 3

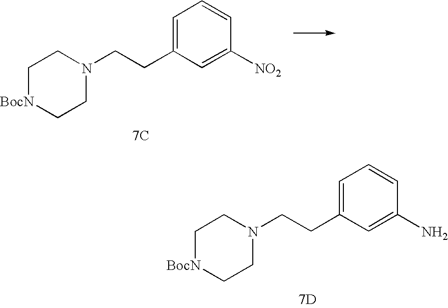

A Parr glass liner was charged with tert-butyl 4-(3-nitrophenethyl)piper-azine-1-carboxylate (7C, 1.0 eq) and methanol (about 0.2 M 7C in MeOH). To this solution was added a slurry of 12.5 wt eq of 10% Pd/C in methanol. The reaction mixture was sealed in a Parr hydrogenation vessel and subjected to 3 pressurization/venting cycles with H₂. The reaction mixture was allowed to proceed at room temperature and 45 psi H₂ for 2.5 h. The reaction mixture was then charged with 12.5 wt eq of Pd(OH)₂/C and the vessel was repressurized with hydrogen (45 psi). After 1 hr, the reaction mixture was filtered through a pad of diatomaceous earth, the diatomaceous earth washed with MeOH, and the combine organic layers concentrated in vacuo to provide the desired tert-butyl 4-(3-aminophenethyl)piperazine-1-carboxylate (7D, 63%), which was used without further purification.

### EXAMPLE 7

### Step 4

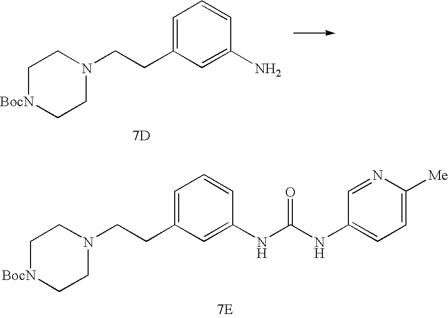

To a solution of tert-butyl 4-(3-aminophenethyl)piperazine-1-carboxylate (7D, 1.0 eq) in THF (about 0.3 M 7D in THF) was added 5-isocyanato-2-methylpyridine (1.0 eq) dropwise. The resulting reaction mixture was stirred for 2 h. To the reaction mixture was added saturated aq. NaHCO₃. The mixture was diluted with EtOAc, and the layers were separated. The organic layer was washed twice with saturated aq. NaHCO₃ and once with brine. The organic layer was dried over Na₂SO₄, filtered and concentrated in vacuo. Purification over silica gel using 5-12% MeOH/CH₂Cl₂ as the gradient eluant provided tert-butyl 4-(3-(3-(6-methylpyridin-3-yl)ureido)phenethyl)piperazine-1-carboxylate (7E, 63%).

### EXAMPLE 7

### Step 5

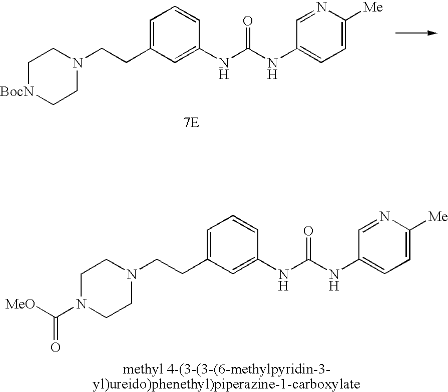

To a solution of tert-butyl 4-(3-(3-(6-methylpyridin-3-yl)ureido)phenethyl)piperazine-1-carboxylate (7E, 1.0 eq) in MeOH (about 0.2 M 7E in MeOH)) was added a solution of 2 M HCl in dioxane (about 12 eq). After 70 min the reaction mixture was concentrated in vacuo and used without purification for subsequent acylations. MS 398 (M+H).

The resulting HCl salt (1.0 eq) from the preceding step was suspended in THF (about 0.15 M salt in THF) and triethylamine (4.0 eq) was added. The reaction mixture was cooled to 0° C., and methyl chloroformate (1.05 eq) was added dropwise and the resultant mixture stirred for 5 min at RT. To the reaction mixture was added saturated aq. NaHCO₃ followed by EtOAc. The layers were separated, and the organic layer was washed once with saturated aq. NaHCO₃, once with brine, dried over Na₂SO₄, filtered and concentrated in vacuo. Purification over silica gel using 2-10% MeOH/CH₂Cl₂ as the gradient eluant afforded methyl 4-(3-(3-(6-methylpyridin-3-yl)ureido)phenethyl)piperazine-1-carboxylate.

### EXAMPLE 8

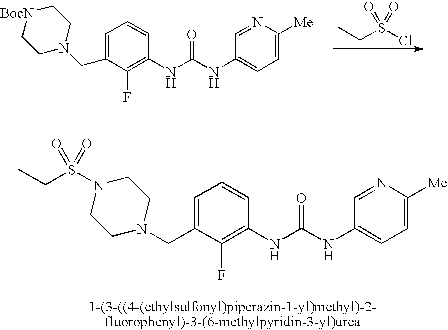

To a solution of 1.0 eq 8A in MeOH (about 0.07 M) was added a solution of 2 M HCl in dioxane (about 30 eq)). After 70 min the reaction mixture was concentrated in vacuo and used without purification for subsequent acylations.

The resulting HCl salt from the preceding step was suspended in THF (about 0.05 M) and about 18 eq diisopropylethylamine was added. The reaction mixture was cooled to 0° C., and about 1 eq ethanesulfonyl chloride was added dropwise. The resultant mixture was stirred for 5 min at RT. To the reaction mixture was added saturated aq. NaHCO₃ followed by EtOAc. The layers were separated, and the organic layer was washed once with saturated aq. NaHCO₃, once with brine, dried over Na₂SO₄, filtered and concentrated in vacuo. Purification over silica gel using 1-10% MeOH/CH₂Cl₂ as the gradient eluant followed by trituration in 1:1 actone/ether afforded methyl 1-(3-((4-(ethylsulfonyl)piperazin-1-yl)methyl)-2-fluorophenyl)-3-(6-methylpyridin-3-yl)urea. MS 436 (M+H).

### EXAMPLE 9

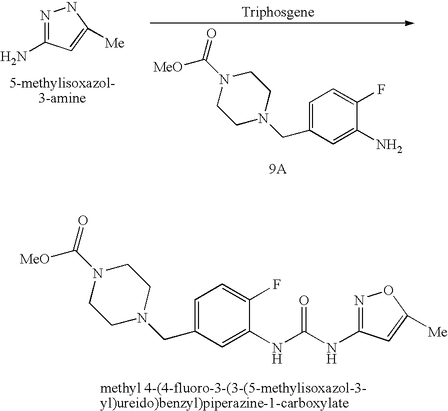

To a solution of about 0.4 eq triphosgene in THF (about 0.04 M) at RT under N₂ atmosphere was added 1 eq 5-methylisoxazol-3-amine and 2 eq diisopropylethylamine in THF (about 0.2 M amine in THF). The reaction mixture was stirred for 15 min. To this mixture was added 1.0 eq 9A in THF (about 0.2 mM 9A in THF). The resultant mixture was stirred for 10 min. To the reaction mixture was added saturated aq. NaHCO₃ followed by EtOAc. The layers were separated, and the organic layer was washed once with saturated aq. NaHCO₃, once with brine, dried over Na₂SO₄, filtered and concentrated in vacuo. Purification over silica gel using 1-10% MeOH/CH₂Cl₂ as the gradient eluant afforded methyl 4-(4-fluoro-3-(3-(5-methylisoxazol-3-yl)ureido)benzyl)piperazine-1-carboxylate. MS 392 (M+H).

The following compounds were synthesized in a manner similar to the representative compounds above:

| Mass Spec | |
|---|---|
| data | Compound Name |
| 347 (M + H) | N-[(3-fluoro-5-{[(6-methyl(3- |
| | pyridyl))amino]carbonylamino}phenyl)methyl]methoxy-N-methylcarboxamide |
| 382 (M + H) | N-[3-({[(dimethylamino)sulfonyl]methylamino}methyl)-5-fluorophenyl](3- |
| | pyridylamino)carboxamide |
| 396 (M + H) | N-[3-({[(dimethylamino)sulfonyl]methylamino}methyl)-5-fluorophenyl][(6- |
| | methyl(3-pyridyl))amino]carboxamide |
| 381 (M + H) | N-(3-{[(ethylsulfonyl)methylamino]methyl}-5-fluorophenyl)[(6-methyl(3- |
| | pyridyl))amino]carboxamide |
| 388 (M + H) | methyl 4-({3-fluoro-5-[(3- |
| | pyridylamino)carbonylamino]phenyl}methyl)piperazinecarboxylate |
| 422 (M + H) | N-(3-{[4-(ethylsulfonyl)piperazinyl]methyl}-5-fluorophenyl)(3- |
| | pyridylamino)carboxamide |
| 402 (M + H) | methyl 4-[(3-fluoro-5-{[(6-methyl(3- |
| | pyridyl))amino]carbonylamino}phenyl)methyl]piperazinecarboxylate |
| 436 (M + H) | N-(3-{[4-(ethylsulfonyl)piperazinyl]methyl}-5-fluorophenyl)[(6-methyl(3- |
| | pyridyl))amino]carboxamide |
| 451 (M + H) | N-[3-({4-[(dimethylamino)sulfonyl]piperazinyl}methyl)-5-fluorophenyl][(6- |
| | methyl(3-pyridyl))amino]carboxamide |
| 437 (M + H) | N-[3-({4-[(dimethylamino)sulfonyl]piperazinyl}methyl)-5-fluorophenyl](3- |
| | pyridylamino)carboxamide |
| 454 (M + H) | N-[3-({4-[(dimethylamino)sulfonyl]piperazinyl}methyl)-5-fluorophenyl][(4- |
| | fluorophenyl)amino]carboxamide |
| 405 (M + H) | methyl 4-[(3-fluoro-5-{[(4- |
| | fluorophenyl)amino]carbonylamino}phenyl)methyl]piperazinecarboxylate |
| 439 (M + H) | N-(3-{[4-(ethylsulfonyl)piperazinyl]methyl}-5-fluorophenyl)[(4- |
| | fluorophenyl)amino]carboxamide |
| 388 (M + H) | methyl 4-({4-fluoro-3-[(3- |
| | pyridylamino)carbonylamino]phenyl}methyl)piperazinecarboxylate |
| 437 (M + H) | N-[5-({4-[(dimethylamino)sulfonyl]piperazinyl}methyl)-2-fluorophenyl](3- |
| | pyridylamino)carboxamide |
| 436 (M + H) | N-(5-{[4-(ethylsulfonyl)piperazinyl]methyl}-2-fluorophenyl)[(6-methyl(3- |
| | pyridyl))amino]carboxamide |
| 422 (M + H) | N-(5-{[4-(ethylsulfonyl)piperazinyl]methyl}-2-fluorophenyl)(3- |
| | pyridylamino)carboxamide |
| 451 (M + H) | N-([5-({4-[(dimethylamino)sulfonyl]piperazinyl}methyl)-2-fluorophenyl][(6- |
| | methyl(3-pyridyl))amino]carboxamide |
| 402 (M + H) | methyl 4-[(4-fluoro-3-{[(6-methyl(3- |
| | pyridyl))amino]carbonylamino}phenyl)methyl]piperazinecarboxylate |
| 386 (M + H) | N-{3-[(4-acetylpiperazinyl)methyl]-5-fluorophenyl}[(6-methyl(3- |
| | pyridyl))amino]carboxamide |
| 422 (M + H) | N-(5-fluoro-3-{[4-(methylsulfonyl)piperazinyl]methyl}phenyl)[(6-methyl(3- |
| | pyridyl))amino]carboxamide |
| 450 (M + H) | N-[5-fluoro-3-({4-[(methylethyl)sulfonyl]piperazinyl}methyl)phenyl][(6- |
| | methyl(3-pyridyl))amino]carboxamide |
| 416 (M + H) | N-(5-fluoro-3-{[4-(2-methoxyacetyl)piperazinyl]methyl}phenyl)[(6-methyl(3- |
| | pyridyl))amino]carboxamide |
| 450 (M + H) | N-(5-fluoro-3-{[4-(propylsulfonyl)piperazinyl]methyl}phenyl)[(6-methyl(3- |
| | pyridyl))amino]carboxamide |
| 439 (M + H) | N-[3-({4-[(1E)-1-(dimethylamino)-2-cyano-2-azavinyl]piperazinyl}methyl)-5- |
| | fluorophenyl][(6-methyl(3-pyridyl))amino]carboxamide |
| 380 (M + H) | N-{5-fluoro-3-[(5-methyl-1,1-dioxo(1,2,5-thiadiazolidin-2- |
| | yl))methyl]phenyl}(3-pyridylamino)carboxamide |
| 394 (M + H) | N-{5-fluoro-3-[(5-methyl-1,1-dioxo(1,2,5-thiadiazolidin-2- |
| | yl))methyl]phenyl}[(6-methyl(3-pyridyl))amino]carboxamide |
| 397 (M + H) | N-{5-fluoro-3-[(5-methyl-1,1-dioxo(1,2,5-thiadiazolidin-2- |
| | yl))methyl]phenyl}[(4-fluorophenyl)amino]carboxamide |
| 402 (M + H) | methyl 4-[(2-fluoro-5-{[(6-methyl(3- |
| | pyridyl))amino]carbonylamino}phenyl)methyl]piperazinecarboxylate |
| 436 (M + H) | N-(3-{[4-(ethylsulfonyl)piperazinyl]methyl}-4-fluorophenyl)[(6-methyl(3- |
| | pyridyl))amino]carboxamide |
| 451 (M + H) | N-[3-({4-[(dimethylamino)sulfonyl]piperazinyl}methyl)-4-fluorophenyl][(6- |
| | methyl(3-pyridyl))amino]carboxamide |
| 388 (M + H) | methyl 4-({2-fluoro-5-[(3- |
| | pyridylamino)carbonylamino]phenyl}methyl)piperazinecarboxylate |
| 422 (M + H) | N-(3-{[4-(ethylsulfonyl)piperazinyl]methyl}-4-fluorophenyl)(3- |
| | pyridylamino)carboxamide |
| 437 (M + H) | N-[3-({4-[(dimethylamino)sulfonyl]piperazinyl}methyl)-4-fluorophenyl](3- |
| | pyridylamino)carboxamide |
| 370 (M + H) | methyl 4-({3-[(3- |
| | pyridylamino)carbonylamino]phenyl}methyl)piperazinecarboxylate |
| 404 (M + H) | N-(3-{[4-(ethylsulfonyl)piperazinyl]methyl}phenyl)(3- |
| | pyridylamino)carboxamide |
| 418 (M + H) | N-(3-{[4-(ethylsulfonyl)piperazinyl]methyl}phenyl)[(6-methyl(3- |
| | pyridyl))amino]carboxamide |
| 384 (M + H) | methyl 4-[(3-{[(6-methyl-3- |
| | pyridyl)amino]carbonylamino}phenyl)methyl]piperazinecarboxylate |
| 419 (M + H) | N-[3-({4-[(dimethylamino)sulfonyl]piperazinyl}methyl)phenyl](3- |
| | pyridylamino)carboxamide |
| 433 (M + H) | N-[3-({4-[(dimethylamino)sulfonyl]piperazinyl}methyl)phenyl][(6-methyl(3- |
| | pyridyl))amino]carboxamide |
| 341 (M + H) | N-{5-fluoro-3-[(3-methyl-2-oxoimidazolidinyl)methyl]phenyl}(3- |
| | pyridylamino)carboxamide |
| 355 (M + H) | N-{5-fluoro-3-[(3-methyl-2-oxoimidazolidinyl)methyl]phenyl}[(6-methyl(3- |
| | pyridyl))amino]carboxamide |
| 358 (M + H) | N-{5-fluoro-3-[(4-methyl-3-oxopiperazinyl)methyl]phenyl}(3- |
| | pyridylamino)carboxamide |
| 343 (M + H+) | N-[3-fluoro-5-(piperidylmethyl)phenyl][(6-methyl(3- |
| | pyridyl))amino]carboxamide |
| 329 (M + H+) | N-[3-fluoro-5-(piperidylmethyl)phenyl](3-pyridylamino)carboxamide |
| 481 (M + H+) | N-[3-({(3S)-4-[(dimethylamino)sulfonyl]-3- |
| | (methoxymethyl)piperazinyl}methyl)-5-fluorophenyl](3- |
| | pyridylamino)carboxamide |
| 466 (M + H) | N-(3-{[(3S)-4-(ethylsulfonyl)-3-(methoxymethyl)piperazinyl]methyl}-5- |
| | fluorophenyl)(3-pyridylamino)carboxamide |
| 432 (M + H) | methyl (2S)-4-({5-fluoro-3-[(3-pyridylamino)carbonylamino]phenyl}methyl)-2- |
| | (methoxymethyl)piperazinecarboxylate |
| 495 (M + H) | N-[3-({(3S)-4-[(dimethylamino)sulfonyl]-3- |
| | (methoxymethyl)piperazinyl}methyl)-5-fluorophenyl][(6-methyl(3- |
| | pyridyl))amino]carboxamide |
| 480 (M + H) | N-(3-{[(3S)-4-(ethylsulfonyl)-3-(methoxymethyl)piperazinyl]methyl}-5- |
| | fluorophenyl)[(6-methyl(3-pyridyl))amino]carboxamide |
| 446 (M + H) | methyl (2S)-4-[(5-fluoro-3-{[(6-methyl(3- |
| | pyridyl))amino]carbonylamino}phenyl)methyl]-2- |
| | (methoxymethyl)piperazinecarboxylate |
| 345 (M + H) | N-[5-fluoro-3-(morpholin-4-ylmethyl)phenyl][(6-methyl(3- |
| | pyridyl))amino]carboxamide |
| 331 (M + H) | N-[5-fluoro-3-(morpholin-4-ylmethyl)phenyl](3-pyridylamino)carboxamide |
| 393 (M + H) | N-{3-[(1,1-dioxo(1,4-thiazaperhydroin-4-yl))methyl]-5-fluorophenyl}[(6- |
| | methyl(3-pyridyl))amino]carboxamide |
| 379 (M + H) | N-{3-[(1,1-dioxo(1,4-thiazaperhydroin-4-yl))methyl]-5-fluorophenyl}(3- |
| | pyridylamino)carboxamide |
| 358 (M + H) | N-{5-fluoro-3-[(4-methylpiperazinyl)methyl]phenyl}[(6-methyl(3- |
| | pyridyl))amino]carboxamide |
| 344 (M + H) | N-{5-fluoro-3-[(4-methylpiperazinyl)methyl]phenyl}(3- |
| | pyridylamino)carboxamide |
| 451 (M + H) | N-{3-[((3S)-3-{[(dimethylamino)sulfonyl]methylamino}pyrrolidinyl)methyl]-5- |
| | fluorophenyl}(3-pyridylamino)carboxamide |
| 436 (M + H) | N-[3-({(3S)-3-[(ethylsulfonyl)methylamino]pyrrolidinyl}methyl)-5- |
| | fluorophenyl](3-pyridylamino)carboxamide |
| 402 (M + H) | N-[(3S)-1-({3-fluoro-5-[(3- |
| | pyridylamino)carbonylamino]phenyl}methyl)pyrrolidin-3-yl]methoxy-N- |
| | methylcarboxamide |
| 465 (M + H) | N-{3-[((3S)-3-{[(dimethylamino)sulfonyl]methylamino}pyrrolidinyl)methyl]-5- |
| | fluorophenyl}[(6-methyl(3-pyridyl))amino]carboxamide |
| 450 (M + H) | N-[3-({(3S)-3-[(ethylsulfonyl)methylamino]pyrrolidinyl}methyl)-5- |
| | fluorophenyl][(6-methyl(3-pyridyl))amino]carboxamide |
| 416 (M + H) | N-{(3S)-1-[(3-fluoro-5-{[(6-methyl(3- |
| | pyridyl))amino]carbonylamino}phenyl)methyl]pyrrolidin-3-yl}methoxy-N- |
| | methylcarboxamide |
| 421 (M + H+) | N-(5-fluoro-3-{[4-(methylsulfonyl)piperidyl]methyl}phenyl)[(6-methyl(3- |
| | pyridyl))amino]carboxamide |
| 407 (M + H+) | N-(5-fluoro-3-{[4-(methylsulfonyl)piperidyl]methyl}phenyl)(3- |
| | pyridylamino)carboxamide |
| 423 (M + H) | N-(3-{[4-(ethylsulfonyl)piperazinyl]methyl}-5-fluorophenyl)(pyrimidin-5- |
| | ylamino)carboxamide |
| 438 (M + H) | N-[3-({4-[(dimethylamino)sulfonyl]piperazinyl}methyl)-5- |
| | fluorophenyl](pyrimidin-5-ylamino)carboxamide |
| 401 (M + H) | methyl 1-[(3-fluoro-5-{[(6-methyl(3- |
| | pyridyl))amino]carbonylamino}phenyl)methyl]piperidine-4-carboxylate |
| 387 (M + H) | methyl 1-({3-fluoro-5-[(3- |
| | pyridylamino)carbonylamino]phenyl}methyl)piperidine-4-carboxylate |
| 392 (M + H) | methyl 4-[(3-fluoro-5-{[(5-methylisoxazol-3- |
| | yl)amino]carbonylamino}phenyl)methyl]piperazinecarboxylate |
| 441 (M + H) | N-[3-({4-[(dimethylamino)sulfonyl]piperazinyl}methyl)-5-fluorophenyl][(5- |
| | methylisoxazol-3-yl)amino]carboxamide |
| 426 (M + H) | N-(3-{[4-(ethylsulfonyl)piperazinyl]methyl}-5-fluorophenyl)[(5- |
| | methylisoxazol-3-yl)amino]carboxamide |
| 465 (M + H) | ({5-[((3R)-3-{[(dimethylamino)sulfonyl]methylamino}piperidyl)methyl]-3- |
| | fluorophenyl}amino)-N-(3-pyridyl)carboxamide |
| 450 (M + H) | {[5-({(3R)-3-[(ethylsulfonyl)methylamino]piperidyl}methyl)-3- |
| | fluorophenyl]amino}-N-(3-pyridyl)carboxamide |
| 416 (M + H) | N-[(3R)-1-({5-fluoro-3-[(N-(3-pyridyl)carbamoyl)amino]phenyl}methyl)(3- |
| | piperidyl)]methoxy-N-methylcarboxamide |
| 479 (M + H) | ({5-[((3R)-3-{[(dimethylamino)sulfonyl]methylamino}piperidyl)methyl]-3- |
| | fluorophenyl}amino)-N-(6-methyl(3-pyridyl))carboxamide |
| 464 (M + H) | {[5-({(3R)-3-[(ethylsulfonyl)methylamino]piperidyl}methyl)-3- |
| | fluorophenyl]amino}-N-(6-methyl(3-pyridyl))carboxamide |
| 430 (M + H) | N-{(3R)-1-[(5-fluoro-3-{[N-(6-methyl(3- |
| | pyridyl))carbamoyl]amino}phenyl)methyl](3-piperidyl)}methoxy-N- |
| | methylcarboxamide |
| 378 (M + H) | methyl 4-({3-fluoro-5-[(isoxazol-3- |
| | ylamino)carbonylamino]phenyl}methyl)piperazinecarboxylate |
| 412 (M + H) | N-(3-{[4-(ethylsulfonyl)piperazinyl]methyl}-5-fluorophenyl)(isoxazol-3- |
| | ylamino)carboxamide |
| 427 (M + H) | N-[3-({4-[(dimethylamino)sulfonyl]piperazinyl}methyl)-5- |
| | fluorophenyl](isoxazol-3-ylamino)carboxamide |
| 450 (M + H) | N-[5-fluoro-3-({4-[methyl(methylsulfonyl)amino]piperidyl}methyl)phenyl][(6- |
| | methyl(3-pyridyl))amino]carboxamide |
| 462 (M − H) | N-[3-({4-[(ethylsulfonyl)methylamino]piperidyl}methyl)-5-fluorophenyl][(6- |
| | methyl(3-pyridyl))amino]carboxamide |
| 479 (M + H) | N-{3-[(4-{[(dimethylamino)sulfonyl]methylamino}piperidyl)methyl]-5- |
| | fluorophenyl}[(6-methyl(3-pyridyl))amino]carboxamide |
| 430 (M + H) | N-{1-[(3-fluoro-5-{[(6-methyl(3- |
| | pyridyl))amino]carbonylamino}phenyl)methyl](4-piperidyl)}methoxy-N- |
| | methylcarboxamide |
| 414 (M + H) | N-{1-[(3-fluoro-5-{[(6-methyl(3- |
| | pyridyl))amino]carbonylamino}phenyl)methyl](4-piperidyl)}-N- |
| | methylacetamide |
| 403 (M + H) | methyl 4-[(3-fluoro-5-{[(2-methylpyrimidin-5- |
| | yl)amino]carbonylamino}phenyl)methyl]piperazinecarboxylate |
| 436 (M + H) | N-[5-fluoro-3-({4-[methyl(methylsulfonyl)amino]piperidyl}methyl)phenyl](3- |
| | pyridylamino)carboxamide |
| 448 (M + H) | N-[3-({4-[(ethylsulfonyl)methylamino]piperidyl}methyl)-5-fluorophenyl](3- |
| | pyridylamino)carboxamide |
| 465 (M + H) | N-{3-[(4-{[(dimethylamino)sulfonyl]methylamino}piperidyl)methyl]-5- |
| | fluorophenyl}(3-pyridylamino)carboxamide |
| 416 (M + H) | N-[1-({3-fluoro-5-[(3-pyridylamino)carbonylamino]phenyl}methyl)(4- |
| | piperidyl)]methoxy-N-methylcarboxamide |
| 400 (M + H) | N-[1-({3-fluoro-5-[(3-pyridylamino)carbonylamino]phenyl}methyl)(4- |
| | piperidyl)]-N-methylacetamide |
| 453 (M + H) | N-[5-fluoro-3-({4-[methyl(methylsulfonyl)amino]piperidyl}methyl)phenyl][(4- |
| | fluorophenyl)amino]carboxamide |
| 467 (M + H) | N-[3-({4-[(ethylsulfonyl)methylamino]piperidyl}methyl)-5-fluorophenyl][(4- |
| | fluorophenyl)amino]carboxamide |
| 482 (M + H) | N-{3-[(4-{[(dimethylamino)sulfonyl]methylamino}piperidyl)methyl]-5- |
| | fluorophenyl}[(4-fluorophenyl)amino]carboxamide |
| 433 (M + H) | N-{1-[(3-fluoro-5-{[(4-fluorophenyl)amino]carbonylamino}phenyl)methyl](4- |
| | piperidyl)}methoxy-N-methylcarboxamide |
| 417 (M + H) | N-{1-[(3-fluoro-5-{[(4-fluorophenyl)amino]carbonylamino}phenyl)methyl](4- |
| | piperidyl)}-N-methylacetamide |
| 472 (M + H) | (tert-butoxy)-N-{1-[(3-fluoro-5-{[(6-methyl(3- |
| | pyridyl))amino]carbonylamino}phenyl)methyl](4-piperidyl)}-N- |
| | methylcarboxamide |
| 458 (M + H) | (tert-butoxy)-N-[1-({3-fluoro-5-[(3- |
| | pyridylamino)carbonylamino]phenyl}methyl)(4-piperidyl)]-N- |
| | methylcarboxamide |
| 475 (M + H) | (tert-butoxy)-N-{1-[(3-fluoro-5-{[(4- |
| | fluorophenyl)amino]carbonylamino}phenyl)methyl](4-piperidyl)}-N- |
| | methylcarboxamide |
| 371 (M + H) | N-(5-fluoro-3-{[4-(methylamino)piperidyl]methyl}phenyl)[(6-methyl(3- |
| | pyridyl))amino]carboxamide |
| 356 (M + H) | N-(5-fluoro-3-{[4-(methylamino)piperidyl]methyl}phenyl)(3- |
| | pyridylamino)carboxamide |
| 378 (M + H) | methyl 4-({4-fluoro-3-[(1,3-oxazol-2- |
| | ylamino)carbonylamino]phenyl}methyl)piperazinecarboxylate |
| 392 (M + H) | methyl 4-[(4-fluoro-3-{[(5-methylisoxazol-3- |
| | yl)amino]carbonylamino}phenyl)methyl]piperazinecarboxylate |
| 403 (M + H) | methyl 4-[(4-fluoro-3-{[(2-methylpyrimidin-5- |
| | yl)amino]carbonylamino}phenyl)methyl]piperazinecarboxylate |
| 391 (M + H) | methyl 4-[(4-fluoro-3-{[(1-methylpyrazol-3- |
| | yl)amino]carbonylamino}phenyl)methyl]piperazinecarboxylate |
| 391 (M − H) | 1-[(3-fluoro-5-{[(6-methyl(3- |
| | pyridyl))amino]carbonylamino}phenyl)methyl]piperidine-4-carboxylic acid |
| 379 (M − H) | 1-({3-fluoro-5-[(3-pyridylamino)carbonylamino]phenyl}methyl)piperidine-4- |
| | carboxylic acid |
| 345 (M + H) | N-[2-fluoro-5-(morpholin-4-ylmethyl)phenyl][(6-methyl(3- |
| | pyridyl))amino]carboxamide |
| 339 (M + H) | methyl 4-({4-fluoro-3-[(pyrimidin-5- |
| | ylamino)carbonylamino]phenyl}methyl)piperazinecarboxylate |
| 430 (M + H) | N-{(3R)-1-[(4-fluoro-3-{[N-(6-methyl(3- |
| | pyridyl))carbamoyl]amino}phenyl)methyl](3-piperidyl)}methoxy-N- |
| | methylcarboxamide |
| 444 (M + H) | N-{(3R)-1-[(4-fluoro-3-{[N-(6-methyl(3- |
| | pyridyl))carbamoyl]amino}phenyl)methyl](3-piperidyl)}ethoxy-N- |
| | methylcarboxamide |
| 458 (M + H) | N-{(3R)-1-[(4-fluoro-3-{[N-(6-methyl(3- |
| | pyridyl))carbamoyl]amino}phenyl)methyl](3-piperidyl)}-N- |
| | methyl(methylethoxy)carboxamide |
| 414 (M + H) | N-{(3R)-1-[(4-fluoro-3-{[N-(6-methyl(3- |
| | pyridyl))carbamoyl]amino}phenyl)methyl](3-piperidyl)}-N-methylacetamide |
| 428 (M + H) | N-{(3R)-1-[(4-fluoro-3-{[N-(6-methyl(3- |
| | pyridyl))carbamoyl]amino}phenyl)methyl](3-piperidyl)}-N-methylpropanamide |
| 442 (M + H) | N-{(3R)-1-[(4-fluoro-3-{[N-(6-methyl(3- |
| | pyridyl))carbamoyl]amino}phenyl)methyl](3-piperidyl)}-2-methyl-N- |
| | methylpropanamide |
| 393 (M + H) | methyl 4-[(4-fluoro-3-{[(5-methyl(1,3,4-oxadiazol-2- |
| | yl))amino]carbonylamino}phenyl)methyl]piperazinecarboxylate |
| 392 (M + H) | methyl 4-[(4-fluoro-3-{[(4-methyl(1,3-oxazol-2- |
| | yl))amino]carbonylamino}phenyl)methyl]piperazinecarboxylate |
| 418 (M + H) | methyl 4-[(4-chloro-3-{[(6-methyl(3- |
| | pyridyl))amino]carbonylamino}phenyl)methyl]piperazinecarboxylate |
| 416 (M + H) | ethyl 4-[(4-fluoro-3-{[(6-methyl(3- |
| | pyridyl))amino]carbonylamino}phenyl)methyl]piperazinecarboxylate |
| 430 (M + H) | methylethyl 4-[(4-fluoro-3-{[(6-methyl(3- |
| | pyridyl))amino]carbonylamino}phenyl)methyl]piperazinecarboxylate |
| 386 (M + H) | N-{5-[(4-acetylpiperazinyl)methyl]-2-fluorophenyl}[(6-methyl(3- |
| | pyridyl))amino]carboxamide |
| 400 (M + H) | N-{2-fluoro-5-[(4-propanoylpiperazinyl)methyl]phenyl}[(6-methyl(3- |
| | pyridyl))amino]carboxamide |
| 414 (M + H) | N-(2-fluoro-5-{[4-(2-methylpropanoyl)piperazinyl]methyl}phenyl)[(6- |
| | methyl(3-pyridyl))amino]carboxamide |
| 458 (M + H) | N-[5-({(3R)-3-[(tert-butoxy)-N-methylcarbonylamino]pyrrolidinyl}methyl)-2- |
| | fluorophenyl][(6-methyl(3-pyridyl))amino]carboxamide |
| 358 (M + H) | N-(5-{[(3R)-3-(methylamino)pyrrolidinyl]methyl}-2-fluorophenyl)[(6- |
| | methyl(3-pyridyl))amino]carboxamide |
| 416 (M + H) | N-(5-{[(3R)-3-(methoxy-N-methylcarbonylamino)pyrrolidinyl]methyl}-2- |
| | fluorophenyl)[(6-methyl(3-pyridyl))amino]carboxamide |
| 430 (M + H) | N-(5-{[(3R)-3-(ethoxy-N-methylcarbonylamino)pyrrolidinyl]methyl}-2- |
| | fluorophenyl)[(6-methyl(3-pyridyl))amino]carboxamide |
| 444 (M + H) | N-[5-({(3R)-3-[N-methyl(methylethoxy)carbonylamino]pyrrolidinyl}methyl)-2- |
| | fluorophenyl][(6-methyl(3-pyridyl))amino]carboxamide |
| 400 (M + H) | N-{(3R)-1-[(4-fluoro-3-{[(6-methyl(3- |
| | pyridyl))amino]carbonylamino}phenyl)methyl]pyrrolidin-3-yl}-N- |
| | methylacetamide |
| 414 (M + H) | N-(5-{[4-(N,N-dimethylcarbamoyl)piperidyl]methyl}-3-fluorophenyl)[(6- |
| | methyl(3-pyridyl))amino]carboxamide |
| 400 (M + H) | N-(3-fluoro-5-{[4-(N-methylcarbamoyl)piperidyl]methyl}phenyl)[(6-methyl(3- |
| | pyridyl))amino]carboxamide |
| 472 (M + H) | N-{(3S)-1-[(4-fluoro-3-{[(6-methyl(3- |
| | pyridyl))amino]carbonylamino}phenyl)methyl](3-piperidyl)}(tert-butoxy)-N- |
| | methylcarboxamide |
| 398 (M + H) | methyl 4-[(4-methyl-3-{[(6-methyl(3- |
| | pyridyl))amino]carbonylamino}phenyl)methyl]piperazinecarboxylate |
| 488 (M + H) | tert-butyl (2S)-4-[(4-fluoro-3-{[(6-methyl(3- |
| | pyridyl))amino]carbonylamino}phenyl)methyl]-2- |
| | (methoxymethyl)piperazinecarboxylate |
| 446 (M + H) | methyl (2S)-4-[(4-fluoro-3-{[(6-methyl(3- |
| | pyridyl))amino]carbonylamino}phenyl)methyl]-2- |
| | (methoxymethyl)piperazinecarboxylate |
| 460 (M + H) | ethyl (2S)-4-[(4-fluoro-3-{[(6-methyl(3- |
| | pyridyl))amino]carbonylamino}phenyl)methyl]-2- |
| | (methoxymethyl)piperazinecarboxylate |
| 474 (M + H) | methylethyl (2S)-4-[(4-fluoro-3-{[(6-methyl(3- |
| | pyridyl))amino]carbonylamino}phenyl)methyl]-2- |
| | (methoxymethyl)piperazinecarboxylate |
| 430 (M + H) | N-(5-{[(3S)-4-acetyl-3-(methoxymethyl)piperazinyl]methyl}-2- |
| | fluorophenyl)[(6-methyl(3-pyridyl))amino]carboxamide |
| 444 (M + H) | N-(5-{[(3S)-3-(methoxymethyl)-4-propanoylpiperazinyl]methyl}-2- |
| | fluorophenyl)[(6-methyl(3-pyridyl))amino]carboxamide |
| 458 (M + H) | N-(5-{[(3S)-3-(methoxymethyl)-4-(2-methylpropanoyl)piperazinyl]methyl}-2- |
| | fluorophenyl)[(6-methyl(3-pyridyl))amino]carboxamide |
| 416 (M + H) | N-(5-{[(3S)-3-(methoxy-N-methylcarbonylamino)pyrrolidinyl]methyl}-2- |
| | fluorophenyl)[(6-methyl(3-pyridyl))amino]carboxamide |
| 430 (M + H) | N-(5-{[(3S)-3-(ethoxy-N-methylcarbonylamino)pyrrolidinyl]methyl}-2- |
| | fluorophenyl)[(6-methyl(3-pyridyl))amino]carboxamide |
| 444 (M + H) | N-[5-({(3S)-3-[N-methyl(methylethoxy)carbonylamino]pyrrolidinyl}methyl)-2- |
| | fluorophenyl][(6-methyl(3-pyridyl))amino]carboxamide |
| 400 (M + H) | N-{(3S)-1-[(4-fluoro-3-{[(6-methyl(3- |
| | pyridyl))amino]carbonylamino}phenyl)methyl]pyrrolidin-3-yl}-N- |
| | methylacetamide |
| 414 (M + H) | N-{(3S)-1-[(4-fluoro-3-{[(6-methyl(3- |
| | pyridyl))amino]carbonylamino}phenyl)methyl]pyrrolidin-3-yl}-N- |
| | methylpropanamide |
| 428 (M + H) | N-{(3S)-1-[(4-fluoro-3-{[(6-methyl(3- |
| | pyridyl))amino]carbonylamino}phenyl)methyl]pyrrolidin-3-yl}-2-methyl-N- |
| | methylpropanamide |
| 430 (M + H) | N-(2-fluoro-5-{[4-(methoxy-N- |
| | methylcarbonylamino)piperidyl]methyl}phenyl)[(6-methyl(3- |
| | pyridyl))amino]carboxamide |
| 444 (M + H) | N-(5-{[4-(ethoxy-N-methylcarbonylamino)piperidyl]methyl}-2- |
| | fluorophenyl)[(6-methyl(3-pyridyl))amino]carboxamide |
| 458 (M + H) | N-[2-fluoro-5-({4-[N- |
| | methyl(methylethoxy)carbonylamino]piperidyl}methyl)phenyl][(6-methyl(3- |
| | pyridyl))amino]carboxamide |
| 414 (M + H) | N-{1-[(4-fluoro-3-{[(6-methyl(3- |
| | pyridyl))amino]carbonylamino}phenyl)methyl](4-piperidyl)}-N- |
| | methylacetamide |
| 428 (M + H) | N-{1-[(4-fluoro-3-{[(6-methyl(3- |
| | pyridyl))amino]carbonylamino}phenyl)methyl](4-piperidyl)}-N- |
| | methylpropanamide |
| 442 (M + H) | N-{1-[(4-fluoro-3-{[(6-methyl(3- |
| | pyridyl))amino]carbonylamino}phenyl)methyl](4-piperidyl)}-2-methyl-N- |
| | methylpropanamide |
| 414 (M + H) | N-{(3R)-1-[(4-fluoro-3-{[(6-methyl(3- |
| | pyridyl))amino]carbonylamino}phenyl)methyl]pyrrolidin-3-yl}-N- |
| | methylpropanamide |
| 428 (M + H) | N-{(3R)-1-[(4-fluoro-3-{[(6-methyl(3- |
| | pyridyl))amino]carbonylamino}phenyl)methyl]pyrrolidin-3-yl}-2-methyl-N- |
| | methylpropanamide |
| 373 (M + H) | N-{5-[((3S,5R)-3,5-dimethylmorpholin-4-yl)methyl]-2-fluorophenyl}[(6- |
| | methyl(3-pyridyl))amino]carboxamide |
| 430 (M + H) | N-{(3S)-1-[(4-fluoro-3-{[(6-methyl(3- |
| | pyridyl))amino]carbonylamino}phenyl)methyl](3-piperidyl)}methoxy-N- |
| | methylcarboxamide |
| 444 (M + H) | N-{(3S)-1-[(4-fluoro-3-{[(6-methyl(3- |
| | pyridyl))amino]carbonylamino}phenyl)methyl](3-piperidyl)}ethoxy-N- |
| | methylcarboxamide |
| 458 (M + H) | N-{(3S)-1-[(4-fluoro-3-{[(6-methyl(3- |
| | pyridyl))amino]carbonylamino}phenyl)methyl](3-piperidyl)}-N- |
| | methyl(methylethoxy)carboxamide |
| 444 (M + H) | tert-butyl 4-[(4-fluoro-3-{[(6-methyl(3- |
| | pyridyl))amino]carbonylamino}phenyl)methyl]piperazinecarboxylate |
| 414 (M + H) | N-{(3S)-1-[(4-fluoro-3-{[(6-methyl(3- |
| | pyridyl))amino]carbonylamino}phenyl)methyl](3-piperidyl)}-N- |
| | methylacetamide |
| 344 (M + H) | N-[2-fluoro-5-(piperazinylmethyl)phenyl][(6-methyl(3- |
| | pyridyl))amino]carboxamide |
| 446 (M + H) | methyl (2R)-4-[(4-fluoro-3-{[(6-methyl(3- |
| | pyridyl))amino]carbonylamino}phenyl)methyl]-2- |
| | (methoxymethyl)piperazinecarboxylate |
| 430 (M + H) | N-(5-{[(3R)-4-acetyl-3-(methoxymethyl)piperazinyl]methyl}-2- |
| | fluorophenyl)[(6-methyl(3-pyridyl))amino]carboxamide |
| 460 (M + H) | ethyl (2R)-4-[(4-fluoro-3-{[(6-methyl(3- |
| | pyridyl))amino]carbonylamino}phenyl)methyl]-2- |
| | (methoxymethyl)piperazinecarboxylate |
| 474 (M + H) | methylethyl (2R)-4-[(4-fluoro-3-{[(6-methyl(3- |
| | pyridyl))amino]carbonylamino}phenyl)methyl]-2- |
| | (methoxymethyl)piperazinecarboxylate |
| 466 (M + H) | N-(5-{[(3R)-3-(methoxymethyl)-4-(methylsulfonyl)piperazinyl]methyl}-2- |
| | fluorophenyl)[(6-methyl(3-pyridyl))amino]carboxamide |
| 372 (M + H) | N-(5-{[(3S)-3-(methylamino)piperidyl]methyl}-2-fluorophenyl)[(6-methyl(3- |
| | pyridyl))amino]carboxamide |
| 428 (M + H) | N-{(3S)-1-[(4-fluoro-3-{[(6-methyl(3- |
| | pyridyl))amino]carbonylamino}phenyl)methyl](3-piperidyl)}-N- |
| | methylpropanamide |
| 442 (M + H) | N-{(3S)-1-[(4-fluoro-3-{[(6-methyl(3- |
| | pyridyl))amino]carbonylamino}phenyl)methyl](3-piperidyl)}-2-methyl-N- |
| | methylpropanamide |
| 458 (M + H) | tert-butyl 4-[(4-fluoro-3-{[(6-methyl(3- |
| | pyridyl))amino]carbonylamino}phenyl)methyl]-1,4- |
| | diazaperhydroepinecarboxylate |
| 400 (M + H) | N-(3-{[4-(N,N-dimethylcarbamoyl)piperidyl]methyl}-5-fluorophenyl)(3- |
| | pyridylamino)carboxamide |
| 389 (M + H) | methyl 4-({4-fluoro-3-[(pyridazin-4- |
| | ylamino)carbonylamino]phenyl}methyl)piperazinecarboxylate |
| 480 (M + H) | N-(5-{[(3R)-4-(ethylsulfonyl)-3-(methoxymethyl)piperazinyl]methyl}-2- |
| | fluorophenyl)[(6-methyl(3-pyridyl))amino]carboxamide |
| 386 (M + H) | N-(5-fluoro-3-{[4-(N-methylcarbamoyl)piperidyl]methyl}phenyl)(3- |
| | pyridylamino)carboxamide |
| 378 (M + H) | methyl 4-({4-fluoro-3-[(isoxazol-3- |
| | ylamino)carbonylamino]phenyl}methyl)piperazinecarboxylate |
| 400 (M + H) | N-{3-[((1S)-7-oxo-8-oxa-3,6-diazabicyclo[4.3.0]non-3-yl)methyl]-5- |
| | fluorophenyl}[(6-methyl(3-pyridyl))amino]carboxamide |
| 400 (M + H) | N-{5-[((1S)-7-oxo-8-oxa-3,6-diazabicyclo[4.3.0]non-3-yl)methyl]-2- |
| | fluorophenyl}[(6-methyl(3-pyridyl))amino]carboxamide |
| 416 (M + H) | methyl 4-[(5-fluoro-3-{[(6-methyl(3- |
| | pyridyl))amino]carbonylamino}phenyl)ethyl]piperazinecarboxylate |
| 430 (M + H) | ethyl 4-[(5-fluoro-3-{[(6-methyl(3- |
| | pyridyl))amino]carbonylamino}phenyl)ethyl]piperazinecarboxylate |
| 400 (M + H) | N-{3-[(4-acetylpiperazinyl)ethyl]-5-fluorophenyl}[(6-methyl(3- |
| | pyridyl))amino]carboxamide |
| 358 (M + H) | N-[5-(1,4-diazaperhydroepinylmethyl)-2-fluorophenyl][(6-methyl(3- |
| | pyridyl))amino]carboxamide |
| 416 (M + H) | methyl 4-[(4-fluoro-3-{[(6-methyl(3- |
| | pyridyl))amino]carbonylamino}phenyl)methyl]-1,4- |
| | diazaperhydroepinecarboxylate |
| 430 (M + H) | ethyl 4-[(4-fluoro-3-{[(6-methyl(3- |
| | pyridyl))amino]carbonylamino}phenyl)methyl]-1,4- |
| | diazaperhydroepinecarboxylate |
| 444 (M + H) | methylethyl 4-[(4-fluoro-3-{[(6-methyl(3- |
| | pyridyl))amino]carbonylamino}phenyl)methyl]-1,4- |
| | diazaperhydroepinecarboxylate |
| 400 (M + H) | N-{5-[(4-acetyl(1,4-diazaperhydroepinyl))methyl]-2-fluorophenyl}[(6- |
| | methyl(3-pyridyl))amino]carboxamide |
| 401 (M + H) | N-{5-[(1,4-dioxa-8-azaspiro[4.5]dec-8-yl)methyl]-2-fluorophenyl}[(6- |
| | methyl(3-pyridyl))amino]carboxamide |
| 373 (M + H) | N-{2-fluoro-5-[(4-methoxypiperidyl)methyl]phenyl}[(6-methyl(3- |
| | pyridyl))amino]carboxamide |
| 357 (M + H) | N-[5-(azaperhydroepinylmethyl)-2-fluorophenyl][(6-methyl(3- |
| | pyridyl))amino]carboxamide |
| 426 (M + H) | N-{2-fluoro-5-[(4-piperidylpiperidyl)methyl]phenyl}[(6-methyl(3- |
| | pyridyl))amino]carboxamide |
| 455 (M + H) | N-(5-{[4-(cyclohexylmethoxy)piperidyl]methyl}-2-fluorophenyl)[(6-methyl(3- |
| | pyridyl))amino]carboxamide |
| 375 (M + H) | N-(2-fluoro-5-{[2-(hydroxymethyl)morpholin-4-yl]methyl}phenyl)[(6- |
| | methyl(3-pyridyl))amino]carboxamide |
| 389 (M + H) | N-(2-fluoro-5-{[2-(methoxymethyl)morpholin-4-yl]methyl}phenyl)[(6- |
| | methyl(3-pyridyl))amino]carboxamide |
| 420 (M + H) | methyl 4-[(2,4-difluoro-5-{[(6-methyl(3- |
| | pyridyl))amino]carbonylamino}phenyl)methyl]piperazinecarboxylate |
| 401 (M + H) | N-{2-fluoro-5-[(4-propoxypiperidyl)methyl]phenyl}[(6-methyl(3- |
| | pyridyl))amino]carboxamide |
| 357 (M + H) | N-{2-fluoro-5-[(4-methylpiperidyl)methyl]phenyl}[(6-methyl(3- |
| | pyridyl))amino]carboxamide |
| 465 (M + H) | N-[5-({4-[(dimethylamino)sulfonyl](1,4-diazaperhydroepinyl)}methyl)-2- |
| | fluorophenyl][(6-methyl(3-pyridyl))amino]carboxamide |
| 444 (M + H) | propyl 4-[(4-fluoro-3-{[(6-methyl(3- |
| | pyridyl))amino]carbonylamino}phenyl)methyl]-1,4- |
| | diazaperhydroepinecarboxylate |
| 400 (M + H) | N-{3-[((1R)-7-oxo-8-oxa-3,6-diazabicyclo[4.3.0]non-3-yl)methyl]-5- |
| | fluorophenyl}[(6-methyl(3-pyridyl))amino]carboxamide |
| 436 (M + H) | N-(2-fluoro-5-{[4-(methylsulfonyl)(1,4- |
| | diazaperhydroepinyl)]methyl}phenyl)[(6-methyl(3-pyridyl))amino]carboxamide |
| 449 (M + H) | N-{3-[((1R)-8-methyl-7,7-dioxo-7-thia-3,6,8-triazabicyclo[4.3.0]non-3- |
| | yl)methyl]-5-fluorophenyl}[(6-methyl(3-pyridyl))amino]carboxamide |
| 450 (M + H) | N-(5-{[4-(ethylsulfonyl)(1,4-diazaperhydroepinyl)]methyl}-2-fluorophenyl)[(6- |
| | methyl(3-pyridyl))amino]carboxamide |
| 449 (M + H) | N-{5-[((1R)-8-methyl-7,7-dioxo-7-thia-3,6,8-triazabicyclo[4.3.0]non-3- |
| | yl)methyl]-2-fluorophenyl}[(6-methyl(3-pyridyl))amino]carboxamide |
| 465 (M + H) | N-[2-fluoro-5-({4-[(methylethyl)sulfonyl](1,4- |
| | diazaperhydroepinyl)}methyl)phenyl][(6-methyl(3-pyridyl))amino]carboxamide |
| 449 (M + H) | N-{3-[((1S)-8-methyl-7,7-dioxo-7-thia-3,6,8-triazabicyclo[4.3.0]non-3- |
| | yl)methyl]-5-fluorophenyl}[(6-methyl(3-pyridyl))amino]carboxamide |
| 449 (M + H) | N-{5-[((1S)-8-methyl-7,7-dioxo-7-thia-3,6,8-triazabicyclo[4.3.0]non-3- |
| | yl)methyl]-2-fluorophenyl}[(6-methyl(3-pyridyl))amino]carboxamide |
| 400 (M + H) | N-{5-[((1R)-7-oxo-8-oxa-3,6-diazabicyclo[4.3.0]non-3-yl)methyl]-2- |
| | fluorophenyl}[(6-methyl(3-pyridyl))amino]carboxamide |
| 418 (M + H) | methyl 4-[(4-fluoro-3-{[(6-methoxy(3- |
| | pyridyl))amino]carbonylamino}phenyl)methyl]piperazinecarboxylate |
| 402 (M + H) | N-{5-[((1R)-7-oxo-8-oxa-3,6-diazabicyclo[4.3.0]non-3-yl)methyl]-2- |
| | fluorophenyl}[(6-methyl(3-pyridyl))amino]carboxamide |
| 438 (M + H) | methyl 4-[(2,4,5-trifluoro-3-{[(6-methyl(3- |
| | pyridyl))amino]carbonylamino}phenyl)methyl]piperazinecarboxylate |
| 451 (M + H) | N-[2-fluoro-5-({4-[methyl(methylsulfonyl)amino]piperidyl}methyl)phenyl][(6- |
| | methyl(3-pyridyl))amino]carboxamide |
| 414 (M + H) | N-{3-[3-(4-acetylpiperazinyl)propyl]-5-fluorophenyl}[(6-methyl(3- |
| | pyridyl))amino]carboxamide |
| 430 (M + H) | methyl 4-[3-(3-fluoro-5-{[(6-methyl(3- |
| | pyridyl))amino]carbonylamino}phenyl)propyl]piperazinecarboxylate |
| 475 (M + H) | (tert-butoxy)-N-{1-[(4-fluoro-3-{[(4- |
| | fluorophenyl)amino]carbonylamino}phenyl)methyl](4-piperidyl)}-N- |
| | methylcarboxamide |
| 475 (M + H) | N-(2-fluoro-5-{[4-(methylamino)piperidyl]methyl}phenyl)[(4- |
| | fluorophenyl)amino]carboxamide |
| 413 (M + H) | methyl 4-[(3-{[(6-cyano(3-pyridyl))amino]carbonylamino}-5- |
| | fluorophenyl)methyl]piperazinecarboxylate |
| 427 (M + H) | ethyl 4-[(3-{[(6-cyano(3-pyridyl))amino]carbonylamino}-5- |
| | fluorophenyl)methyl]piperazinecarboxylate |
| 441 (M + H) | methylethyl 4-[(3-{[(6-cyano(3-pyridyl))amino]carbonylamino}-5- |
| | fluorophenyl)methyl]piperazinecarboxylate |
| 397 (M + H) | N-{3-[(4-acetylpiperazinyl)methyl]-5-fluorophenyl}[(6-cyano(3- |
| | pyridyl))amino]carboxamide |
| 462 (M + H) | N-[3-({4-[(dimethylamino)sulfonyl]piperazinyl}methyl)-5-fluorophenyl][(6- |
| | cyano(3-pyridyl))amino]carboxamide |
| 447 (M + H) | [(6-cyano(3-pyridyl))amino]-N-(3-{[4-(ethylsulfonyl)piperazinyl]methyl}-5- |
| | fluorophenyl)carboxamide |
| 453 (M + H) | N-[2-fluoro-5-({4-[methyl(methylsulfonyl)amino]piperidyl}methyl)phenyl][(4- |
| | fluorophenyl)amino]carboxamide |
| 467 (M + H) | N-[5-({4-[(ethylsulfonyl)methylamino]piperidyl}methyl)-2-fluorophenyl][(4- |
| | fluorophenyl)amino]carboxamide |
| 458 (M + H) | tert-butyl (3S)-3-{[(4-fluoro-3-{[(6-methyl(3- |
| | pyridyl))amino]carbonylamino}phenyl)methyl]methylamino}pyrrolidinecarboxylate |
| 416 (M + H) | methyl (3S)-3-{[(4-fluoro-3-{[(6-methyl(3- |
| | pyridyl))amino]carbonylamino}phenyl)methyl]methylamino}pyrrolidinecarboxylate |
| 416 (M + H) | methyl (3R)-3-{[(4-fluoro-3-{[(6-methyl(3- |
| | pyridyl))amino]carbonylamino}phenyl)methyl]methylamino}pyrrolidinecarboxylate |
| 398 (M + H) | methyl 4-[(2-methyl-3-{[(6-methyl(3- |
| | pyridyl))amino]carbonylamino}phenyl)methyl]piperazinecarboxylate |
| 418 (M + H) | methyl 4-[(2-chloro-5-{[(6-methyl(3- |
| | pyridyl))amino]carbonylamino}phenyl)methyl]piperazinecarboxylate |
| 429 (M + H) | 2-{4-[(3-fluoro-5-{[(6-methyl(3- |
| | pyridyl))amino]carbonylamino}phenyl)methyl]piperazinyl}-N,N- |
| | dimethylacetamide |
| 444 (M + H) | ethyl 4-[(3-{[(6-acetyl(3-pyridyl))amino]carbonylamino}-5- |
| | fluorophenyl)methyl]piperazinecarboxylate |
| 400 (M + H) | N-{3-[3-(4-acetylpiperazinyl)propyl]-5-fluorophenyl}(3- |
| | pyridylamino)carboxamide |
| 416 (M + H) | methyl 4-(3-{3-fluoro-5-[(3- |
| | pyridylamino)carbonylamino]phenyl}propyl)piperazinecarboxylate |
| 450 (M + H) | N-(3-{3-[4-(ethylsulfonyl)piperazinyl]propyl}-5-fluorophenyl)(3- |
| | pyridylamino)carboxamide |
| 444 (M + H) | ethyl 4-[3-(3-fluoro-5-{[(6-methyl(3- |
| | pyridyl))amino]carbonylamino}phenyl)propyl]piperazinecarboxylate |
| 458 (M + H) | methylethyl 4-[3-(3-fluoro-5-{[(6-methyl(3- |
| | pyridyl))amino]carbonylamino}phenyl)propyl]piperazinecarboxylate |
| 464 (M + H) | N-(3-{3-[4-(ethylsulfonyl)piperazinyl]propyl}-5-fluorophenyl)[(6-methyl(3- |
| | pyridyl))amino]carboxamide |
| 479 (M + H) | N-[3-(3-{4-[(dimethylamino)sulfonyl]piperazinyl}propyl)-5-fluorophenyl][(6- |
| | methyl(3-pyridyl))amino]carboxamide |
| 430 (M + H) | N-{3-[3-(4-acetylpiperazinyl)propyl]-5-fluorophenyl}[(6-methoxy(3- |
| | pyridyl))amino]carboxamide |
| 446 (M + H) | methyl 4-[3-(3-fluoro-5-{[(6-methoxy(3- |
| | pyridyl))amino]carbonylamino}phenyl)propyl]piperazinecarboxylate |
| 480 (M + H) | N-(3-{3-[4-(ethylsulfonyl)piperazinyl]propyl}-5-fluorophenyl)[(6-methoxy(3- |
| | pyridyl))amino]carboxamide |
| 430 (M + H) | methyl 4-[(3-{[(6-acetyl(3-pyridyl))amino]carbonylamino}-5- |
| | fluorophenyl)methyl]piperazinecarboxylate |
| 358 (M + H) | N-(5-{[((3S)pyrrolidin-3-yl)methylamino]methyl}-2-fluorophenyl)[(6-methyl(3- |
| | pyridyl))amino]carboxamide |
| 458 (M + H) | tert-butyl (3R)-3-{[(4-fluoro-3-{[(6-methyl(3- |
| | pyridyl))amino]carbonylamino}phenyl)methyl]methylamino}pyrrolidinecarboxylate |
| 358 (M + H) | N-(5-{[((3R)pyrrolidin-3-yl)methylamino]methyl}-2-fluorophenyl)[(6- |
| | methyl(3-pyridyl))amino]carboxamide |
| 444 (M + H) | N-ethyl-N-{1-[(4-fluoro-3-{[(6-methyl(3- |
| | pyridyl))amino]carbonylamino}phenyl)methyl](4- |
| | piperidyl)}methoxycarboxamide |
| 458 (M + H) | ethoxy-N-ethyl-N-{1-[(4-fluoro-3-{[(6-methyl(3- |
| | pyridyl))amino]carbonylamino}phenyl)methyl](4-piperidyl)}carboxamide |
| 478 (M + H) | N-[5-({4-[ethyl(ethylsulfonyl)amino]piperidyl}methyl)-2-fluorophenyl][(6- |
| | methyl(3-pyridyl))amino]carboxamide |
| 428 (M + H) | N-ethyl-N-{1-[(4-fluoro-3-{[(6-methyl(3- |
| | pyridyl))amino]carbonylamino}phenyl)methyl](4-piperidyl)}acetamide |
| 413 (M + H) | methyl 4-[(3-{[(6-cyano(3-pyridyl))amino]carbonylamino}-4- |
| | fluorophenyl)methyl]piperazinecarboxylate |
| 427 (M + H) | ethyl 4-[(3-{[(6-cyano(3-pyridyl))amino]carbonylamino}-4- |
| | fluorophenyl)methyl]piperazinecarboxylate |
| 441 (M + H) | methylethyl 4-[(3-{[(6-cyano(3-pyridyl))amino]carbonylamino}-4- |
| | fluorophenyl)methyl]piperazinecarboxylate |
| 397 (M + H) | N-{5-[(4-acetylpiperazinyl)methyl]-2-fluorophenyl}[(6-cyano(3- |
| | pyridyl))amino]carboxamide |
| 452 (M + H) | methyl 4-[(3-{[(6-methyl(3-pyridyl))amino]carbonylamino}-5- |
| | (trifluoromethyl)phenyl)methyl]piperazinecarboxylate |
| 398 (M + H) | methyl 4-[(2-methyl-5-{[(6-methyl(3- |
| | pyridyl))amino]carbonylamino}phenyl)methyl]piperazinecarboxylate |
| 420 (M + H) | methyl 4-[(2,6-difluoro-3-{[(6-methyl(3- |
| | pyridyl))amino]carbonylamino}phenyl)methyl]piperazinecarboxylate |
| 436 (M + H) | methyl 4-[(4-chloro-2-fluoro-5-{[(6-methyl(3- |
| | pyridyl))amino]carbonylamino}phenyl)methyl]piperazinecarboxylate |
| 458 (M + H) | tert-butyl 4-[(1R)-1-(5-fluoro-3-{[(6-methyl(3- |
| | pyridyl))amino]carbonylamino}phenyl)ethyl]piperazinecarboxylate |
| 416 (M + H) | methyl 4-[(1R)-1-(5-fluoro-3-{[(6-methyl(3- |
| | pyridyl))amino]carbonylamino}phenyl)ethyl]piperazinecarboxylate |
| 430 (M + H) | ethyl 4-[(1R)-1-(5-fluoro-3-{[(6-methyl(3- |
| | pyridyl))amino]carbonylamino}phenyl)ethyl]piperazinecarboxylate |
| 444 (M + H) | ethyl 4-[(3-{[(6-acetyl(3-pyridyl))amino]carbonylamino}-4- |
| | fluorophenyl)methyl]piperazinecarboxylate |
| 458 (M + H) | methylethyl 4-[(3-{[(6-acetyl(3-pyridyl))amino]carbonylamino}-4- |
| | fluorophenyl)methyl]piperazinecarboxylate |
| 414 (M + H) | [(6-acetyl(3-pyridyl))amino]-N-{5-[(4-acetylpiperazinyl)methyl]-2- |
| | fluorophenyl}carboxamide |
| 430 (M + H) | methyl 4-{[(4-fluoro-3-{[(6-methyl(3- |
| | pyridyl))amino]carbonylamino}phenyl)methyl]methylamino}piperidinecarboxylate |
| 414 (M + H) | N-(5-{[(1-acetyl(4-piperidyl))methylamino]methyl}-2-fluorophenyl)[(6- |
| | methyl(3-pyridyl))amino]carboxamide |
| 464 (M + H) | N-[5-({[1-(ethylsulfonyl)(4-piperidyl)]methylamino}methyl)-2- |
| | fluorophenyl][(6-methyl(3-pyridyl))amino]carboxamide |
| 446 (M + H) | N-{5-[({2-[(tert-butoxy)-N-methylcarbonylamino]ethyl}methylamino)methyl]- |
| | 2-fluorophenyl}[(6-methyl(3-pyridyl))amino]carboxamide |
| 449 (M + H) | N-{5-[({2-[(tert-butoxy)-N-methylcarbonylamino]ethyl}methylamino)methyl]- |
| | 2-fluorophenyl}[(4-fluorophenyl)amino]carboxamide |
| 418 (M + H) | methyl 4-[(2-chloro-3-{[(6-methyl(3- |
| | pyridyl))amino]carbonylamino}phenyl)methyl]piperazinecarboxylate |
| 452 (M + H) | methyl 4-[(3-{[(6-methyl(3-pyridyl))amino]carbonylamino}-4- |
| | (trifluoromethyl)phenyl)methyl]piperazinecarboxylate |
| 458 (M + H) | tert-butyl 4-[(1S)-1-(5-fluoro-3-{[(6-methyl(3- |
| | pyridyl))amino]carbonylamino}phenyl)ethyl]piperazinecarboxylate |
| 416 (M + H) | methyl 4-[(1S)-1-(5-fluoro-3-{[(6-methyl(3- |
| | pyridyl))amino]carbonylamino}phenyl)ethyl]piperazinecarboxylate |
| 430 (M + H) | ethyl 4-[(1S)-1-(5-fluoro-3-{[(6-methyl(3- |
| | pyridyl))amino]carbonylamino}phenyl)ethyl]piperazinecarboxylate |
| 430 (M + H) | methyl 4-[(3-{[(6-acetyl(3-pyridyl))amino]carbonylamino}-4- |
| | fluorophenyl)methyl]piperazinecarboxylate |
| 348 (M + H) | N-[2-fluoro-5-(morpholin-4-ylmethyl)phenyl][(4- |
| | fluorophenyl)amino]carboxamide |
| 346 (M + H) | N-[2-fluoro-5-({methyl[2-(methylamino)ethyl]amino}methyl)phenyl][(6- |
| | methyl(3-pyridyl))amino]carboxamide |
| 349 (M + H) | N-[2-fluoro-5-({methyl[2-(methylamino)ethyl]amino}methyl)phenyl][(4- |
| | fluorophenyl)amino]carboxamide |
| 407 (M + H) | N-(2-{[(4-fluoro-3-{[(4- |
| | fluorophenyl)amino]carbonylamino}phenyl)methyl]methylamino}ethyl)methoxy- |
| | N-methylcarboxamide |
| 391 (M + H) | N-(2-{[(4-fluoro-3-{[(4- |
| | fluorophenyl)amino]carbonylamino}phenyl)methyl]methylamino}ethyl)-N- |
| | methylacetamide |
| 409 (M + H) | methyl 4-[(2-cyano-5-{[(6-methyl(3- |
| | pyridyl))amino]carbonylamino}phenyl)methyl]piperazinecarboxylate |
| 420 (M + H) | methyl 4-[(3,4-difluoro-5-{[(6-methyl(3- |
| | pyridyl))amino]carbonylamino}phenyl)methyl]piperazinecarboxylate |
| 427 (M + H) | N-{2-fluoro-5-[(methyl{2- |
| | [methyl(methylsulfonyl)amino]ethyl}amino)methyl]phenyl}[(4- |
| | fluorophenyl)amino]carboxamide |
| 441 (M + H) | N-{5-[({2-[(ethylsulfonyl)methylamino]ethyl}methylamino)methyl]-2- |
| | fluorophenyl}[(4-fluorophenyl)amino]carboxamide |
| 348 (M + H) | N-[5-fluoro-3-(morpholin-4-ylmethyl)phenyl][(4- |
| | fluorophenyl)amino]carboxamide |
| 404 (M + H) | N-(2-{[(4-fluoro-3-{[(6-methyl(3- |
| | pyridyl))amino]carbonylamino}phenyl)methyl]methylamino}ethyl)methoxy-N- |
| | methylcarboxamide |
| 388 (M + H) | N-(2-{[(4-fluoro-3-{[(6-methyl(3- |
| | pyridyl))amino]carbonylamino}phenyl)methyl]methylamino}ethyl)-N- |
| | methylacetamide |
| 440 (M + H) | tert-butyl 4-[(1S)-1-(3-{[(6-methyl(3- |
| | pyridyl))amino]carbonylamino}phenyl)ethyl]piperazinecarboxylate |
| 340 (M + H) | N-[3-((1S)-1-piperazinylethyl)phenyl][(6-methyl(3-pyridyl))amino]carboxamide |
| 398 (M + H) | methyl 4-[(1S)-1-(3-{[(6-methyl(3- |
| | pyridyl))amino]carbonylamino}phenyl)ethyl]piperazinecarboxylate |
| 412 (M + H) | ethyl 4-[(1S)-1-(3-{[(6-methyl(3- |
| | pyridyl))amino]carbonylamino}phenyl)ethyl]piperazinecarboxylate |
| 432 (M + H) | N-(3-{(1S)-1-[4-(ethylsulfonyl)piperazinyl]ethyl}phenyl)[(6-methyl(3- |
| | pyridyl))amino]carboxamide |
| 382 (M + H) | N-{3-[(1S)-1-(4-acetylpiperazinyl)ethyl]phenyl}[(6-methyl(3- |
| | pyridyl))amino]carboxamide |
| 424 (M + H) | N-{2-fluoro-5-[(methyl{2- |
| | [methyl(methylsulfonyl)amino]ethyl}amino)methyl]phenyl}[(6-methyl(3- |
| | pyridyl))amino]carboxamide |
| 438 (M + H) | N-{5-[({2-[(ethylsulfonyl)methylamino]ethyl}methylamino)methyl]-2- |
| | fluorophenyl}[(6-methyl(3-pyridyl))amino]carboxamide |
| 398 (M + H) | methyl 4-[(1R)-1-(3-{[(6-methyl(3- |
| | pyridyl))amino]carbonylamino}phenyl)ethyl]piperazinecarboxylate |
| 412 (M + H) | ethyl 4-[(1R)-1-(3-{[(6-methyl(3- |
| | pyridyl))amino]carbonylamino}phenyl)ethyl]piperazinecarboxylate |
| 416 (M + H) | methyl 4-[(1S)-1-(2-fluoro-3-{[(6-methyl(3- |
| | pyridyl))amino]carbonylamino}phenyl)ethyl]piperazinecarboxylate |
| 430 (M + H) | ethyl 4-[(1S)-1-(2-fluoro-3-{[(6-methyl(3- |
| | pyridyl))amino]carbonylamino}phenyl)ethyl]piperazinecarboxylate |
| 400 (M + H) | N-{3-[(1S)-1-(4-acetylpiperazinyl)ethyl]-2-fluorophenyl}[(6-methyl(3- |
| | pyridyl))amino]carboxamide |
| 420 (M + H) | methyl 4-[(2,4-difluoro-3-{[(6-methyl(3- |
| | pyridyl))amino]carbonylamino}phenyl)methyl]piperazinecarboxylate |
| 420 (M + H) | methyl 4-[(2,5-difluoro-3-{[(6-methyl(3- |
| | pyridyl))amino]carbonylamino}phenyl)methyl]piperazinecarboxylate |
| 398 (M + H) | methyl 4-[2-(3-{[(6-methyl-3- |
| | pyridyl)amino]carbonylamino}phenyl)ethyl]piperazinecarboxylate |
| 412 (M + H) | ethyl 4-[2-(3-{[(6-methyl-3- |
| | pyridyl)amino]carbonylamino}phenyl)ethyl]piperazinecarboxylate |
| 432 (M + H) | N-(3-{2-[4-(ethylsulfonyl)piperazinyl]ethyl}phenyl)[(6-methyl(3- |
| | pyridyl))amino]carboxamide |
| 430 (M + H) | methyl 4-[3-(2-fluoro-3-{[(6-methyl(3- |
| | pyridyl))amino]carbonylamino}phenyl)propyl]piperazinecarboxylate |
| 472 (M + H) | tert-butyl 4-[3-(2-fluoro-3-{[(6-methyl(3- |
| | pyridyl))amino]carbonylamino}phenyl)propyl]piperazinecarboxylate |
| 400 (M + H) | methyl 4-[(2-hydroxy-3-{[(6-methyl(3- |
| | pyridyl))amino]carbonylamino}phenyl)methyl]piperazinecarboxylate |
| 434 (M + H) | N-(3-{[4-(ethylsulfonyl)piperazinyl]methyl}-2-hydroxyphenyl)[(6-methyl(3- |
| | pyridyl))amino]carboxamide |
| 411 (M + H) | N-(3-{2-[4-(N,N-dimethylcarbamoyl)piperazinyl]ethyl}phenyl)[(6-methyl(3- |
| | pyridyl))amino]carboxamide |
| 447 (M + H) | N-[3-(2-{4-[(dimethylamino)sulfonyl]piperazinyl}ethyl)phenyl][(6-methyl(3- |
| | pyridyl))amino]carboxamide |
| 418 (M + H) | [(6-methyl(3-pyridyl))amino]-N-(3-{2-[4- |
| | (methylsulfonyl)piperazinyl]ethyl}phenyl)carboxamide |
| 414 (M + H) | ethyl 4-[(2-hydroxy-3-{[(6-methyl(3- |
| | pyridyl))amino]carbonylamino}phenyl)methyl]piperazinecarboxylate |
| 420 (M + H) | N-(2-hydroxy-3-{[4-(methylsulfonyl)piperazinyl]methyl}phenyl)[(6-methyl(3- |
| | pyridyl))amino]carboxamide |
| 382 (M + H) | N-{3-[2-(4-acetylpiperazinyl)ethyl]phenyl}[(6-methyl(3- |
| | pyridyl))amino]carboxamide |
| 372 (M + H) | N-[2-fluoro-3-(3-piperazinylpropyl)phenyl][(6-methyl(3- |
| | pyridyl))amino]carboxamide |
| 464 (M + H) | N-(3-{3-[4-(ethylsulfonyl)piperazinyl]propyl}-2-fluorophenyl)[(6-methyl(3- |
| | pyridyl))amino]carboxamide |
| 414 (M + H) | N-{3-[3-(4-acetylpiperazinyl)propyl]-2-fluorophenyl}[(6-methyl(3- |
| | pyridyl))amino]carboxamide |
| 444 (M + H) | ethyl 4-[3-(2-fluoro-3-{[(6-methyl(3- |
| | pyridyl))amino]carbonylamino}phenyl)propyl]piperazinecarboxylate |
| 416 (M + H) | methyl 4-[(3-{[(1-hydroxy-6-methyl-3- |
| | pyridyl)amino]carbonylamino}phenyl)methyl]piperazinecarboxylate |
| 434 (M + H) | methyl 4-[(2-fluoro-3-{[(1-hydroxy-6-methyl(3- |
| | pyridyl))amino]carbonylamino}phenyl)methyl]piperazinecarboxylate |
| 506 (M + H) | phenylmethyl (2S,6R)-4-[(2-fluoro-3-{[(6-methyl(3- |
| | pyridyl))amino]carbonylamino}phenyl)methyl]-2,6- |
| | dimethylpiperazinecarboxylate |
| 414 (M + H) | N-{3-[((3S,5R)-4-acetyl-3,5-dimethylpiperazinyl)methyl]-2-fluorophenyl}[(6- |
| | methyl(3-pyridyl))amino]carboxamide |
| 444 (M + H) | tert-butyl 4-[(2-fluoro-3-{[N-(6-methyl(3- |
| | pyridyl))carbamoyl]amino}phenyl)methyl]piperazinecarboxylate |
| 416 (M + H) | ethyl 4-[(2-fluoro-3-{[N-(6-methyl(3- |
| | pyridyl))carbamoyl]amino}phenyl)methyl]piperazinecarboxylate |
| 386 (M + H) | ({3-[(4-acetylpiperazinyl)methyl]-2-fluorophenyl}amino)-N-(6-methyl(3- |
| | pyridyl))carboxamide |
| 451 (M + H) | {[3-({4-[(dimethylamino)sulfonyl]piperazinyl}methyl)-2-fluorophenyl]amino}- |
| | N-(6-methyl(3-pyridyl))carboxamide |
| 415 (M + H) | [(3-{[4-(N,N-dimethylcarbamoyl)piperazinyl]methyl}-2-fluorophenyl)amino]- |
| | N-(6-methyl(3-pyridyl))carboxamide |
| 436 (M + H) | [(3-{[4-(ethylsulfonyl)piperazinyl]methyl}-2-fluorophenyl)amino]-N-(6- |
| | methyl(3-pyridyl))carboxamide |
| 422 (M + H) | [(2-fluoro-3-{[4-(methylsulfonyl)piperazinyl]methyl}phenyl)amino]-N-(6- |
| | methyl(3-pyridyl))carboxamide |
| 430 (M + H) | methyl (2S,6R)-4-[(2-fluoro-3-{[(6-methyl(3- |
| | pyridyl))amino]carbonylamino}phenyl)methyl]-2,6- |
| | dimethylpiperazinecarboxylate |
| 372 (M + H) | N-{3-[((3S,5R)-3,5-dimethylpiperazinyl)methyl]-2-fluorophenyl}[(6-methyl(3- |
| | pyridyl))amino]carboxamide |
| 392 (M + H) | methyl 4-[(2-fluoro-3-{[(5-methylisoxazol-3- |
| | yl)amino]carbonylamino}phenyl)methyl]piperazinecarboxylate |
| 405 (M + H) | methyl 4-[(2-fluoro-3-{[(4- |
| | fluorophenyl)amino]carbonylamino}phenyl)methyl]piperazinecarboxylate |
| 413 (M + H) | methyl 4-[(3-{[N-(6-cyano(3-pyridyl))carbamoyl]amino}-2- |
| | fluorophenyl)methyl]piperazinecarboxylate |
| 430 (M + H) | methyl 4-[(3-{[N-(6-acetyl(3-pyridyl))carbamoyl]amino}-2- |
| | fluorophenyl)methyl]piperazinecarboxylate |
| 456 (M + H) | methyl 4-{[2-fluoro-3-({N-[6-(trifluoromethyl)(3- |
| | pyridyl)]carbamoyl}amino)phenyl]methyl}piperazinecarboxylate |
| 388 (M + H) | methyl 4-({2-fluoro-3-[(N-(4- |
| | pyridyl)carbamoyl)amino]phenyl}methyl)piperazinecarboxylate |
| 463 (M + H) | [(3-{[4-(azetidinylsulfonyl)piperazinyl]methyl}-2-fluorophenyl)amino]-N-(6- |
| | methyl(3-pyridyl))carboxamide |
| 472 (M + H) | tert-butyl(5S,3R)-4-[(2-fluoro-3-{[N-(6-methyl(3- |
| | pyridyl))carbamoyl]amino}phenyl)methyl]-3,5-dimethylpiperazinecarboxylate |
| 430 (M + H) | methyl (5S,3R)-4-[(2-fluoro-3-{[N-(6-methyl(3- |
| | pyridyl))carbamoyl]amino}phenyl)methyl]-3,5-dimethylpiperazinecarboxylate |
| 414 (M + H) | ({3-[((6S,2R)-4-acetyl-2,6-dimethylpiperazinyl)methyl]-2- |
| | fluorophenyl}amino)-N-(6-methyl(3-pyridyl))carboxamide |
| 443 (M + H) | {(5S,3R)-4-[(2-fluoro-3-{[N-(6-methyl(3- |
| | pyridyl))carbamoyl]amino}phenyl)methyl]-3,5-dimethylpiperazinyl}-N,N- |
| | dimethylcarboxamide |
| 464 (M + H) | [(3-{[(6S,2R)-4-(ethylsulfonyl)-2,6-dimethylpiperazinyl]methyl}-2- |
| | fluorophenyl)amino]-N-(6-methyl(3-pyridyl))carboxamide |
| 479 (M + H) | {[3-({(6S,2R)-4-[(dimethylamino)sulfonyl]-2,6-dimethylpiperazinyl}methyl)-2- |
| | fluorophenyl]amino}-N-(6-methyl(3-pyridyl))carboxamide |
| 382 (M + H) | N-[2-fluoro-3-(1,2,4-triazolo[3,4-c]piperazin-7-ylmethyl)phenyl][(6-methyl(3- |
| | pyridyl))amino]carboxamide |
| 396 (M + H) | N-{2-fluoro-3-[(3-methyl(1,2,4-triazolo[3,4-c]piperazin-7- |
| | yl))methyl]phenyl}[(6-methyl(3-pyridyl))amino]carboxamide |
| 410 (M + H) | N-{3-[(3-ethyl(1,2,4-triazolo[3,4-c]piperazin-7-yl))methyl]-2-fluorophenyl}[(6- |
| | methyl(3-pyridyl))amino]carboxamide |
| 408 (M + H) | N-(2-fluoro-3-{[4-(methylsulfonyl)piperazinyl]methyl}phenyl)(4- |
| | pyridylamino)carboxamide |
| 422 (M + H) | N-(3-{[4-(ethylsulfonyl)piperazinyl]methyl}-2-fluorophenyl)(4- |
| | pyridylamino)carboxamide |
| 402 (M + H) | methyl 4-[(2-fluoro-3-{[(6-methyl(3- |
| | pyridyl))amino]carbonylamino}phenyl)methyl]piperazinecarboxylate |

### EXAMPLE 10

### 1-(2-Chloro-5-((4-(methylsulfonyl)piperazin-1-yl)methyl)benzyl)-3-(6-methylpyridin-3-yl)urea

tert-butyl 4-(4-chloro-3-cyanobenzoyl)piperazine-1-carboxylate. In a 100 mL round-bottom flask under a positive nitrogen pressure of 4-chloro-3-cyanobenzoic acid (4.28 mmol), 1-(tert-butoxycarbonyl)piperazine (1.59 g, 8.56.mmol), EDC (903 mg, 4.71 mmol) and HOBT (694 mg, 5.14 mmol) were dissolved in 17 mL of CH₂Cl₂. Triethylamine (1.5 mL, 10.7 mmol) was added and the reaction mixture was stirred for 20 h. After this time additional quantities of EDC (411 mg, 2.14 mmol), HOBT (290 mg, 2.14 mmol) and triethylamine (300 μL, 2.14 mmol) were added. After stirring for an additional 20 h the reaction mixture was washed with 6 mL portions of: 10% KHSO₄, water, saturated NaHCO₃, and saturated NaCl. The organic extracts were dried over Na₂SO₄, filtered and concentrated to afford 1.44 g of tert-butyl 4-(4-chloro-3-cyanobenzoyl)piperazine-1-carboxylate as a beige solid.

tert-butyl 4-(3-(aminomethyl)-4-chlorobenzyl)piperazine-1-carboxylate. To a 250 mL round-bottom flask fitted with a reflux condenser, rubber septum and stir bar under a positive pressure of N₂ was added 10.3 mL of 1M BH₃□THF complex in THF. This mixture was cooled in an ice bath. The tert-butyl 4-(4-chloro-3-cyanobenzoyl)piperazine-1-carboxylate (1.44 g, 4.12 mmol) was dissolved in 20 mL of THF and added dropwise to the reaction mixture via syringe. The ice bath was removed, replaced with a heating mantle and the reaction mixture was heated at reflux for 20 h. The reaction was cooled to RT and to the mixture was added 48 mL of 20% HOAc in water (v/v) and the mixture was stirred between pH 3-4 for 20 h. The mixture was concentrated to half its original volume with a rotary evaporator and then diluted with 20 mL of 10% citric acid. This mixture was washed once with 25 mL of EtOAc and the aqueous layer was brought to pH=11 by the addition of 52 mL of 3N NaOH. The resultant mixture was extracted with EtOAc (3×40 mL). The combined extracts were washed with 40 mL of saturated NaCl solution. The organic layer was dried over Na₂SO₄, filtered and concentrated to give 1.00 g of tert-butyl 4-(3-(aminomethyl)-4-chlorobenzyl)piperazine-1-carboxylate as a pale yellow oil.

tert-butyl 4-(4-chloro-3-((3-(6-methylpyridin-3-yl)ureido)methyl)benzyl)piperazine-1-carboxylate. The tert-butyl 4-(3-(aminomethyl)-4-chlorobenzyl)piperazine-1-carboxylate from Step 2 (990 mg, 2.91 mmol) was dissolved in 8 mL of CH₂Cl₂ and maintained under a positive nitrogen pressure. 3-Isocyanato-6-methylpyridine (430 mg, 3.20 mmol) was dissolved in 8 mL of CH₂Cl₂ and added dropwise to the tert-butyl 4-(3-(aminomethyl)-4-chlorobenzoyl)piperazine-1-carboxylate solution via syringe. After 15 min, triethylamine was added (410 μL, 2.91 mmol) and stirring was continued for an additional 45 min. After this time the reaction was filtered through a cotton plug to remove the insoluble bis-pyridyl urea. The filtrate was washed with 6 mL portions of water and saturated NaCl solution. The organic layer was dried over Na₂SO₄, filtered and concentrated to afford 1.41 g of a white foam. This material was purified on a silica pad (9.5 cm in diameter, 6 cm high) in a 600 mL fritted glass funnel. Elution was as follows: 1 L of methanol-EtOAc-triethylamine (5:94:1 v/v), 500 mL methanol-EtOAc (10:90 to 25:75 v/v). 250-mL fractions were collected. 958 mg of tert-butyl 4-(4-chloro-3-((3-(6-methylpyridin-3-yl)ureido)methyl)benzyl)piperazine-1-carboxylate as a colorless oil was obtained. TLC methanol-EtOAc-triethylamine (10:89:1 v/v) R_{f}=0.28.

1-(2-chloro-5-(piperazin-1-ylmethyl)benzyl)-3-(6-methylpyridin-3-yl)urea trihydrochloride salt. The tert tert-butyl 4-(4-chloro-3-((3-(6-methylpyridin-3-yl)ureido)methyl)benzyl)piperazine-1-carboxylate from above (954 mg, 2.01 mmol) was dissolved in 40 ml of methanol and treated with 10 mL of 4N HCl in dioxane (40.2.mmol) with stirring for 16 h. The solvents were removed to afford 1.13 g of 1-(2-chloro-5-(piperazin-1-ylmethyl)benzyl)-3-(6-methylpyridin-3-yl)urea trihydrochloride salt as a white solid.

1-(2-chloro-5-((4-(methylsulfonyl)piperazin-1-yl)methyl)benzyl)-3-(6-methylpyridin-3-yl)urea. In a 20 mL scintillation vial equipped with a stir bar, 1-(2-chloro-5-(piperazin-1-ylmethyl)benzyl)-3-(6-methylpyridin-3-yl)urea hydrochloride salt (367 mg, 654 μmol) and DMAP (2 mg) were sealed with a septum cap and maintained under a positive nitrogen pressure. To the mixture was added 6 mL of anhydrous CH₂Cl₂, followed by DIPEA (520 μL, 2.94 mmol) and methanesulfonyl chloride (70 μL, 785 μmol). The reaction mixture was stirred for 16 h. The reaction mixture was diluted with 6 mL of EtOAc. The organic solution was washed with 4 mL each of water and saturated NaCl solution, dried over Na2SO4, filtered and concentrated to afford 1-(2-chloro-5-((4-(methylsulfonyl)piperazin-1-yl)methyl)benzyl)-3-(6-methylpyridin-3-yl)urea as beige foam (209 mg).

### EXAMPLE 11

### Methyl 4-(2-(difluoromethoxy)-5-((3-(6-methylpyridin-3-yl)ureido)methyl)benzyl)piperazine-1-carboxylate

tert-butyl 4-(2-hydroxy-5-iodobenzoyl)piperazine-1-carboxylate. Performed identical to tert-butyl 4-(4-chloro-3-cyanobenzoyl)piperazine-1-carboxylate in Example 1 except 2-hydroxy-5-iodobenzoic acid was used in place of 3-cyano-4-chlorobenzoic acid.

tert-butyl 4-(2-(difluoromethoxy)-5-iodobenzoyl)piperazine-1-carboxylate. A stirred slurry of tert-butyl 4-(2-hydroxy-5-iodobenzoyl)piperazine-1-carboxylate (7.06 g, 16.33 mmol) and potassium hydroxide (30% aqueous, 120 mL) in 2-propanol (200 mL) at 50□C was treated with chlorodifluoromethane by bubbling a stream of the gaseous reagent through the stirring reaction mixture for 6 min. The reaction mixture was placed in a Parr high pressure reaction vessel, heated at 80□C for 16 h and then cooled to ambient temperature. The resulting solution was concentrated to remove 2-propanol and the aqueous portion was extracted with ethyl acetate (3×200 mL). The organic portions were dried (Na₂SO₄) and concentrated. The residue was purified by reverse phase HPLC to give 5.55 g of tert tert-butyl 4-(2-(difluoromethoxy)-5-iodobenzoyl)piperazine-1-carboxylate as a white solid.

tert-butyl 4-(5-cyano-2-(difluoromethoxy)benzoyl)piperazine-1-carboxylate. In a 250 mL round-bottom flask tert-butyl 4-(2-hydroxy-5-iodobenzoyl)piperazine-1-carboxylate (5.55 g, 11.5 mmol), Zn(CN)₂ (2.06 g, 17.2 mmol) and Pd(PPh₃)₄ (1.31 g, 1.15 mmol) were suspended in 93 mL of anhydrous DMF under a positive nitrogen pressure. The reaction mixture was heated for 16 h at 70□C. After this time the addition of the cyanide source and catalyst were repeated. After an additional 16 h of reaction time the mixture was diluted with 280 mL of water and extracted with EtOAc (3×125 mL). The combined organic extracts were dried over Na₂SO₄, filtered and concentrated to afford 5.76 g of a brown oil. The oil was purified by reverse-phase HPLC to afford 2.90 g of tert-butyl 4-(5-cyano-2-(difluoromethoxy)benzoyl)piperazine-1-carboxylate as a white foam.

Methyl 4-(2-(difluoromethoxy)-5-((3-(6-methylpyridin-3-yl)ureido)methyl)benzyl)piperazine-1-carboxylate. The tert-butyl 4-(5-cyano-2-(difluoromethoxy)benzoyl)piperazine-1-carboxylate from above was converted to methyl 4-(2-(difluoromethoxy)-5-((3-(6-methylpyridin-3-yl)ureido)methyl)benzyl)piperazine-1-carboxylate by methods similar to those used in example 1.

### EXAMPLE 12

### Ethyl 4-(4-fluoro-3-((3-(3-methylisoxazol-5-yl)ureido)methyl)benzyl)piperazine-1-carboxylate

tert-butyl 4-(3-cyano-4-fluorobenzyl)piperazine-1-carboxylate: 2-Fluoro-5-formylbenzonitrile (25 g, 167.6 mmol, 1.0 equiv.) was dissolved in CH₂Cl₂ (450 mL) at RT. To this solution was added tert-butyl piperazine-1-carboxylate (31.2 g, 167.6 mmol, 1 equiv.) followed by the portion-wise addition of sodium triacetoxyborohydride (49.7 g, 234.6 mmol, 1.4 equiv.). The reaction vessel was placed under an atmosphere of nitrogen and allowed to stir at room temperature for 1 hour. Saturated NaHCO₃ was added and the resultant mixture stirred for 10 minutes. The mixture was concentrated under reduced pressure, diluted with EtOAc (700 mL), and extracted into 1N KHSO₄ (3×150 mL). The aqueous layer was basified to pH 10 using 50% NaOH solution, saturated with NaCl, extracted into DCM (2×100 mL) and EtOAc (1×200 mL), and dried over Na₂SO₄. Concentration in vacuo resulted in the isolation of 10.2 g of tert-butyl 4-(3-cyano-4-fluorobenzyl)piperazine-1-carboxylate.

Ethyl 4-(3-cyano-4-fluorobenzyl)piperazine-1-carboxylate. The tert-butyl 4-(3-cyano-4-fluorobenzyl)piperazine-1-carboxylate from the previous step (15.0 g, 47.0 mmol, 1.0 equiv.) was dissolved in CH₂Cl₂ (150 mL), to which TFA (150 mL) was slowly added and the resultant mixture stirred for 10 minutes. CH₂Cl₂ and TFA were removed by concentration in vacuo. The resulting residue was dissolved in THF (170 mL) and Et₃N (Aldrich, redistilled, 16.6 mL, 188.1 mmol, 4.0 equiv.). The reaction vessel was cooled to 0° C. and ethyl chloroformate (Aldrich, 4.7 mL, 49.4 mmol, 1.05 equiv.) was added dropwise via syringe under an nitrogen atmosphere. After 30 the reaction was concentrated under reduced pressure, diluted with EtOAc, washed with sat. aq. NaHCO₃ and brine, and dried over Na₂SO₄. The organic layer was removed in vacuo to yield 5.9 g of ethyl 4-(3-cyano-4-fluorobenzyl)piperazine-1-carboxylate.

Ethyl 4-(3-(aminomethyl)-4-fluorobenzyl)piperazine-1-carboxylate. Ethyl 4-(3-cyano-4-fluorobenzyl)piperazine-1-carboxylate (5.9 g, 20.3 mmol, 1.0 equiv.) was dissolved in MeOH (47 mL), to which was added 12 M HCl (2.0 mL, 24.3 mmol, 1.2 equiv) while stirring vigorously. A catalytic amount of palladium on carbon (Aldrich, wet, 10% w/w) was then added as a MeOH slurry. The reaction was placed in a Parr bomb under atmosphere of H₂ (55 psi) for 1 hour at room temperature. The reaction mixture was filtered through Celite and concentrated under reduced pressure to provide 5 g of ethyl 4-(3-(aminomethyl)-4-fluorobenzyl)piperazine-1-carboxylate.

Ethyl 4-(4-fluoro-3-((3-(3-methylisoxazol-5-yl)ureido)methyl)benzyl)piperazine-1-carboxylate. 5-Amino-3-methyl-isoxazole (100 mg, 1.02 mmol, 1.0 equiv.) was added to a vial and dissolved in anhydrous THF (EMD, 5 mL) and redistilled DIPEA (196 ul, 1.12 mmol, 1.1 equiv.) under atmosphere of N₂. To this solution 4-nitrophenyl carbonochloridate (205 mg, 1.02 mmol, 1.0 equiv.) was added directly and the vial was purged again with nitrogen. After 10 minutes, the reaction turned off-white and opaque; after 12 hours of stirring at room temperature the mixture became yellow in color. The presence of intermediate 7 was confirmed by TLC (10% MeOH/DCM) and reverse phase LC/MS: a strong UV peak was observed but the mass was not observed in positive ionization mode. Ethyl 4-(3-(aminomethyl)-4-fluorobenzyl)piperazine-1-carboxylate (139 mg, 1.02 mmol, 1.0 equiv.) in a minimal amount of THF/DCM and added drop-wise to the reaction vessel under atmosphere of N₂. The mixture was stirred for 1 hour then heated to 65° C. for 2 hours. The reaction was allowed to cool to room temperature and then diluted with ethyl acetate (30 mL), washed with 1N NaOH (2×10 mL) and brine (1×10 mL), and dried over Na₂SO₄. The organic layer was then concentrated under reduced pressure to yield a yellow oil. The yellow oil was loaded onto a Biotage samplet and purified via automated silica gel chromatography in MeCN/DCM (Linear gradient from 15% to 74% [300 mL], held at 74%[140 mL], linear gradient from 74% to 100% [300 mL], and held at 100% [400 mL]) to provide 24 mg of ethyl 4-(4-fluoro-3-((3-(3-methylisoxazol-5-yl)ureido)methyl)benzyl)piperazine-1-carboxylate.

### EXAMPLE 13

### (S)—N,N-dimethyl-4-(3-(1-(3-(6-methylpyridin-3-yl)ureido)ethyl)benzyl)piperazine-1-sulfonamide

tert-butyl 4-(3-acetylbenzoyl)piperazine-1-carboxylate. 3-Acetylbenzoic acid (1.64 g, 10.0 mmol), 1-tert-butoxycarbonylpiperazine (2.23 g, 12.0 mmol), HATU (4.56 g, 12.0 mmol) and HOAT (1.63 g, 12.0 mmol) were dissolved in 20 ml anhydrous DMF in a 100 mL round-bottom flask under a positive pressure of N₂ in an ice bath. DIPEA (3.8 mL, 22.0 mmol) was added and the mixture stirred at ambient temperature for 2 h. The solution was diluted with 100 mL of EtOAc and washed with 40 mL each: 0.2N NaOH solution×1 and saturated NaCl solution×2. The organic layer was dried over Na₂SO₄, filtered and was concentrated and purified by silica gel chromatography using EtOAc-hexanes (2:1 v/v) as the eluant to give tert-butyl 4-(3-acetylbenzoyl)piperazine-1-carboxylate as 2.97 g of white solid.

(S)-tert-butyl 4-(3-(1-hydroxyethyl)benzyl)piperazine-1-carboxylate. Under a positive pressure of N₂ 1-1.5M solution of (S)-Methyl oxazaborolidine (0.5 mL, 5.0 mmol) was diluted in 2 mL of and treated with 3.6 mL of borane-N,N-diethylaniline. The tert-butyl 4-(3-acetylbenzoyl)piperazine-1-carboxylate from above in 3 ml of anhydrous toluene was added to the above solution over 1 hour and stirred for an additional 1 h. The mixture was stirred at 20° C. for another 1 hour. To the mixture was added 6 mL MeOH, 12 mL 1N HCl and the resultant mixture stirred for 20 min. The mixture was diluted with 50 mL toluene and washed with saturated NaCl (50 mL). The organic extracts were dried over Na₂SO₄, filtered and concentrated at reduced pressure. The residue was heated at reflux in Et₂NH for 1 hour. Purification by reverse-phase HPLC to afforded (S)-tert-butyl 4-(3-(1-hydroxyethyl)benzyl)piperazine-1-carboxylate as 1.08 g of white solid.

(S)-tert-butyl 4-(3-(1-(1,3-dioxoisoindolin-2-yl)ethyl)benzyl)piperazine-1-carboxylate. To a solution of (S)-tert-butyl 4-(3-(1-hydroxyethyl)benzyl)piperazine-1-carboxylate (480 mg, 1.50 mmol) in 10 mL of anhydrous THF at 0° C. under a positive pressure of N₂ were added phthalimde (330 mg, 2.25 mmol), triphenyl phosphine (590 mg, 2.25 mmol) and DIAD (440 μL, 2.25 mmol). The solution was warmed up to ambient temperature and stirred 1 h. The solvent was removed at reduced pressure and the residue was purified by reverse-phase HPLC to afford (S)-tert-butyl 4-(3-(1-(1,3-dioxoisoindolin-2-yl)ethyl)benzyl)piperazine-1-carboxylate as 490 mg of a white solid.

(S)-tert-butyl 4-(3-(1-aminoethyl)benzyl)piperazine-1-carboxylate. The (S)-tert-butyl 4-(3-(1-(1,3-dioxoisoindolin-2-yl)ethyl)benzyl)piperazine-1-carboxylate from the previous step (490 mg, 1.09 mmol) was dissolved in 5 mL hydrazine and stirred at ambient temperature for 16 h. The solvent was removed at reduced pressure and the resulting (S)-tert-butyl 4-(3-(1-aminoethyl)benzyl)piperazine-1-carboxylate was used in the next step without additional purification.

(S)-tert-butyl 4-(3-(1-(3-(6-methylpyridin-3-yl)ureido)ethyl)benzyl)piperazine-1-carboxylate. (S)-tert-butyl 4-(3-(1-(3-(6-methylpyridin-3-yl)ureido)ethyl)benzyl)piperazine-1-carboxylate was synthesized in a manner analogous to tert-butyl 4-(4-chloro-3-((3-(6-methylpyridin-3-yl)ureido)methyl)benzyl)piperazine-1-carboxylate in example 1.

(S)-1-(6-methylpyridin-3-yl)-3-(1-(3-(piperazin-1-ylmethyl)phenyl)ethyl)urea trihydrochloride salt. (S)-1-(6-methylpyridin-3-yl)-3-(1-(3-(piperazin-1-ylmethyl)phenyl)ethyl)urea trihydrochloride salt was synthesized in a manner analogous to 1-(2-chloro-5-(piperazin-1-ylmethyl)benzyl)-3-(6-methylpyridin-3-yl)urea trihydrochloride salt in example 1.

(S)—N,N-dimethyl-4-(3-(1-(3-(6-methylpyridin-3-yl)ureido)ethyl)benzyl)piperazine-1-sulfonamide. (S)—N,N-dimethyl-4-(3-(1-(3-(6-methylpyridin-3-yl)ureido)ethyl)benzyl)piperazine-1-sulfonamide was synthesized in a manner analogous to 1-(2-chloro-5-((4-(methylsulfonyl)piperazin-1-yl)methyl)benzyl)-3-(6-methylpyridin-3-yl)urea in Example 1 except that N,N-dimethylsulfamoyl chloride was used as the electrophile instead of methanesulfonyl chloride.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the invention.

1.-78. (canceled)

79. A pharmaceutical composition comprising
at least one pharmaceutical agent for the treatment of a disorder chosen from sarcopenia, wasting syndrome, frailty, muscle spasm, cachexia, neuromuscular diseases, and post-surgical and post-traumatic muscle weakness;
at least one compound of Formula IV: 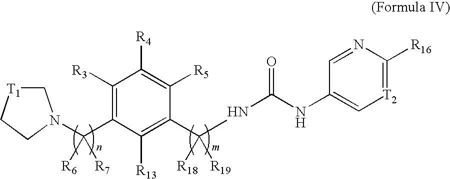
or a pharmaceutically acceptable salt thereof; and
at least one pharmaceutically acceptable excipient, carrier or adjuvant,
wherein:
m is zero, one, two, or three;
n is one, two, or three;
R₃ is chosen from hydrogen, cyano, optionally substituted alkyl, halo, and optionally substituted alkoxy;
R₄ is chosen from hydrogen, pyridinyl, halo, and optionally substituted alkyl;
R₅ is chosen from hydrogen, pyridinyl, halo, optionally substituted alkyl, and optionally substituted alkoxy;
R₆ and R₇ are independently chosen from hydrogen, aminocarbonyl, alkoxycarbonyl, optionally substituted alkyl, and optionally substituted alkoxy;
R₁₃ is chosen from hydrogen, cyano, lower alkyl, hydroxyl, and halo;
R₁₈ and R₁₉ are each independently chosen from hydrogen, aminocarbonyl, alkoxycarbonyl, optionally substituted alkyl, and optionally substituted alkoxy;
T₁ is chosen from —CHR₁₄—, —NR₁₅CHR₁₄—, —CHR₁₄NR₁₅—, and —CHR₁₄CHR₁₄—;
R₁₄ is chosen from hydrogen, methyl, and methoxymethyl;
R₁₅ is chosen from optionally substituted acyl, optionally substituted lower alkoxycarbonyl, and optionally substituted sulfonyl;
T₂ is —C═ or —N═; and
R₁₆ is chosen from hydrogen, halo, cyano, optionally substituted acyl, optionally substituted alkyl, and optionally substituted alkoxy;
wherein the at least one pharmaceutical agent is chosen from sibramine, diethylpropion, phentermine, benzaphetamine, phendimetrazine, estrogen, estradiol, norethindrone acetate, estradiol valerate, ethinyl estradiol, norgestimate, conjugated estrogens, esterified estrogens, testosterone, insulin-derived growth factor, human growth hormone, riluzole, cannabidiol, prednisone, albuterol, non-steroidal anti-inflammatory drugs, botulinum toxin, IGF-1, IGF-2, clonidine, sumatriptan, physostigmine, pyridostigmine, parathyroid hormone, PTH(1-34), tamoxifen, raloxifine, a levongestrel, medroxyprogesterone acetate, orlistat, ATL-962, sibutramine, topiramate, axokine, dexamphetamine, phentermine, phenylpropanolamine, mazindol, indinavir sulfate, saquinavir, saquinavir mesylate, ritonavir, lamivudine, zidovudine, lamivudine/zidovudine combinations, zalcitabine, didanosine, stavudine, and megestrol acetate.

80. The composition of claim 79 wherein T₁ is —NR₁₅CHR₁₄—.

81. The composition of claim 80 wherein R₁₅ is chosen from lower alkoxycarbonyl, lower alkylsulfonyl, and optionally substituted aminosulfonyl.

82. The composition of claim 79 wherein T₂ is —C═.

83. The composition of claim 79 wherein R₆ and R₇ are both hydrogen.

84. The composition of claim 79 wherein R₁₆ is chosen from hydrogen, methyl, fluoro, cyano, methoxy, and acetyl.

85. The composition of claim 79 wherein R₁₈ and R₁₉ are both hydrogen.

86. The composition of claim 79 wherein m is zero.

87. The composition of claim 79 wherein n is one.

88. The composition of claim 79 wherein R₃ is chosen from methyl, ethyl, trifluoromethyl, difluoromethyl, trifluoromethoxy, chloro, fluoro, and hydrogen.

89. The composition of claim 79 wherein R₄ is chosen from hydrogen, methyl, trifluoromethyl and pyridinyl.

90. The composition of claim 79 wherein R₅ is chosen from hydrogen, methyl, chloro, fluoro, difluoromethyl, trifluoromethyl, trifluoromethoxy, difluoromethoxy, and methoxy.

91. The composition of claim 79 wherein R₁₃ is chosen from methyl, ethyl, hydrogen and fluoro.

92. The composition of claim 91 wherein R₁₃ is fluoro.

93. The composition of claim 79 wherein R₁₄ is chosen from hydrogen and methyl.

94. The composition of claim 79 wherein the at least one chemical entity is chosen from:
4-(3-fluoro-5-(3-(6-methylpyridin-3-yl)ureido)benzyl)-N,N-dimethylpiperazine-1-sulfonamide;
N-(1-(3-fluoro-5-(3-(6-methylpyridin-3-yl)ureido)benzyl)piperidin-4-yl)-N-methylethanesulfonamide;
methyl 4-(1-(3-fluoro-5-(3-(6-methylpyridin-3-yl)ureido)phenyl)ethyl)piperazine-1-carboxylate;
ethyl 4-(1-(3-fluoro-5-(3-(6-methylpyridin-3-yl)ureido)phenyl)ethyl)piperazine-1-carboxylate;
methyl 4-(2-fluoro-3-(3-(6-methylpyridin-3-yl)ureido)benzyl)piperazine-1-carboxylate;
methyl 4-(3-(3-fluoro-5-(3-(6-methylpyridin-3-yl)ureido)phenyl)propyl)piperazine-1-carboxylate;
1-(3-(3-(4-(ethylsulfonyl)piperazin-1-yl)propyl)-5-fluorophenyl)-3-(6-methylpyridin-3-yl)urea;
4-(3-(3-fluoro-5-(3-(6-methylpyridin-3-yl)ureido)phenyl)propyl)-N,N-dimethylpiperazine-1-sulfonamide;
methyl 4-(3-(3-(6-methylpyridin-3-yl)ureido)-5-(trifluoromethyl)benzyl)piperazine-1-carboxylate;
methyl 4-(3-(3-(6-methylpyridin-3-yl)ureido)-4-(trifluoromethyl)benzyl)piperazine-1-carboxylate;
(R)-ethyl 4-(1-(3-fluoro-5-(3-(6-methylpyridin-3-yl)ureido)phenyl)ethyl)piperazine-1-carboxylate;
(S)-tert-butyl 4-(1-(3-(3-(6-methylpyridin-3-yl)ureido)phenyl)ethyl)piperazine-1-carboxylate;
(S)-methyl 4-(1-(2-fluoro-3-(3-(6-methylpyridin-3-yl)ureido)phenyl)ethyl)piperazine-1-carboxylate;
(S)-1-(3-(1-(4-acetylpiperazin-1-yl)ethyl)-2-fluorophenyl)-3-(6-methylpyridin-3-yl)urea;
methyl 4-(2,5-difluoro-3-(3-(6-methylpyridin-3-yl)ureido)benzyl)piperazine-1-carboxylate;
methyl 4-(3-(2-fluoro-3-(3-(6-methylpyridin-3-yl)ureido)phenyl)propyl)piperazine-1-carboxylate;
methyl 4-(2-hydroxy-3-(3-(6-methylpyridin-3-yl)ureido)benzyl)piperazine-1-carboxylate;
ethyl 4-(2-hydroxy-3-(3-(6-methylpyridin-3-yl)ureido)benzyl)piperazine-1-carboxylate;
1-(3-(3-(4-acetylpiperazin-1-yl)propyl)-2-fluorophenyl)-3-(6-methylpyridin-3-yl)urea;
ethyl 4-(3-(2-fluoro-3-(3-(6-methylpyridin-3-yl) ureido)phenyl)propyl)piperazine-1-carboxylate;
tert-butyl 4-(2-fluoro-3-(3-(6-methylpyridin-3-yl)ureido)benzyl)piperazine-1-carboxylate;
ethyl 4-(2-fluoro-3-(3-(6-methylpyridin-3-yl)ureido)benzyl)piperazine-1-carboxylate;
1-(3-((4-acetylpiperazin-1-yl)methyl)-2-fluorophenyl)-3-(6-methylpyridin-3-yl)urea;
4-(2-fluoro-3-(3-(6-methylpyridin-3-yl)ureido)benzyl)-N,N-dimethylpiperazine-1-sulfonamide;
4-(2-fluoro-3-(3-(6-methylpyridin-3-yl)ureido)benzyl)-N,N-dimethylpiperazine-1-carboxamide;
1-(3-((4-(ethylsulfonyl)piperazin-1-yl)methyl)-2-fluorophenyl)-3-(6-methylpyridin-3-yl)urea;
1-(2-fluoro-3-((4-(methylsulfonyl)piperazin-1-yl)methyl)phenyl)-3-(6-methylpyridin-3-yl)urea;
methyl 4-(2-fluoro-3-(3-(4-fluorophenyl)ureido)benzyl)piperazine-1-carboxylate;
methyl 4-(3-(3-(6-cyanopyridin-3-yl)ureido)-2-fluorobenzyl)piperazine-1-carboxylate;
methyl 4-(3-(3-(6-acetylpyridin-3-yl)ureido)-2-fluorobenzyl)piperazine-1-carboxylate;
methyl 4-(2-fluoro-3-(3-(6-(trifluoromethyl)pyridin-3-yl)ureido)benzyl)piperazine-1-carboxylate;
1-(3-((4-(azetidin-1-ylsulfonyl)piperazin-1-yl)methyl)-2-fluorophenyl)-3-(6-methylpyridin-3-yl)urea;
tert-butyl 4-(4-fluoro-3-((3-(6-methylpyridin-3-yl)ureido)methyl)benzyl)piperazine-1-carboxylate;
methyl 4-(4-fluoro-3-((3-(6-methylpyridin-3-yl)ureido)methyl)benzyl)piperazine-1-carboxylate;
ethyl 4-(4-fluoro-3-((3-(6-methylpyridin-3-yl)ureido)methyl)benzyl)piperazine-1-carboxylate;
1-(5-((4-acetylpiperazin-1-yl)methyl)-2-fluorobenzyl)-3-(6-methylpyridin-3-yl)urea;
1-(5-((4-(ethylsulfonyl)piperazin-1-yl)methyl)-2-fluorobenzyl)-3-(6-methylpyridin-3-yl)urea;
4-(4-fluoro-3-((3-(6-methylpyridin-3-yl)ureido)methyl)benzyl)-N,N-dimethylpiperazine-1-sulfonamide;
4-(2-chloro-5-((3-(6-methylpyridin-3-yl)ureido)methyl)benzyl)-N,N-dimethylpiperazine-1-sulfonamide;
N,N-dimethyl-4-(2-methyl-5-((3-(6-methylpyridin-3-yl)ureido)methyl)benzyl)piperazine-1-sulfonamide;
methyl 4-(4-(difluoromethoxy)-3-((3-(6-methylpyridin-3-)ureido)methyl)benzyl)piperazine-1-carboxylate;
ethyl 4-(4-(difluoromethoxy)-3-((3-(6-methylpyridin-3-yl)ureido)methyl)benzyl)piperazine-1-carboxylate;
1-(2-fluoro-5-((4-(methylsulfonyl)piperazin-1-yl)methyl)benzyl)-3-(6-methylpyridin-3-yl)urea;
isopropyl 4-(4-fluoro-3-((3-(6-methylpyridin-3-yl)ureido)methyl)benzyl)piperazine-1-carboxylate;
1-(2-fluoro-5-((4-(isopropylsulfonyl)piperazin-1-yl)methyl)benzyl)-3-(6-methylpyridin-3-yl)urea;
1-(2-fluoro-5-((4-(3-methylbutanoyl)piperazin-1-yl)methyl)benzyl)-3-(6-methylpyridin-3-yl)urea;
1-(2-fluoro-5-((4-(propylsulfonyl)piperazin-1-yl)methyl)benzyl)-3-(6-methylpyridin-3-yl)urea;
1-(2-fluoro-5-((4-pivaloylpiperazin-1-yl)methyl)benzyl)-3-(6-methylpyridin-3-yl)urea;
methyl 4-(2-(difluoromethoxy)-5-((3-(6-methylpyridin-3-yl)ureido)methyl)benzyl)piperazine-1-carboxylate;
ethyl 4-(2-(difluoromethoxy)-5-((3-(6-methylpyridin-3-yl)ureido)methyl)benzyl)piperazine-1-carboxylate;
1-(4-(difluoromethoxy)-3-((4-(ethylsulfonyl)piperazin-1-yl)methyl)benzyl)-3-(6-methylpyridin-3-yl)urea;
1-(4-(difluoromethoxy)-3-((4-(methylsulfonyl)piperazin-1-yl)methyl)benzyl)-3-(6-methylpyridin-3-yl)urea;
ethyl 4-(3-((3-(6-acetylpyridin-3-yl)ureido)methyl)-4-fluorobenzyl)piperazine-1-carboxylate;
ethyl 4-(4-methyl-3-((3-(6-methylpyridin-3-yl)ureido)methyl)benzyl)piperazine-1-carboxylate;
isopropyl 4-(4-methyl-3-((3-(6-methylpyridin-3-yl)ureido)methyl)benzyl)piperazine-1-carboxylate;
1-(5-((4-(ethylsulfonyl)piperazin-1-yl)methyl)-2-methylbenzyl)-3-(6-methylpyridin-3-yl)urea;
1-(5-((4-acetylpiperazin-1-yl)methyl)-2-methylbenzyl)-3-(6-methylpyridin-3-yl)urea;
1-(5-((4-(isopropylsulfonyl)piperazin-1-yl)methyl)-2-methylbenzyl)-3-(6-methylpyridin-3-yl)urea;
1-(5-((4-isobutyrylpiperazin-1-yl)methyl)-2-methylbenzyl)-3-(6-methylpyridin-3-yl)urea;
ethyl 4-(2,4-difluoro-3-((3-(6-methylpyridin-3-yl)ureido)methyl)benzyl)piperazine-1-carboxylate;
1-(6-cyanopyridin-3-yl)-3-(5-((4-(ethylsulfonyl)piperazin-1-yl)methyl)-2-fluorobenzyl)urea;
1-(6-acetylpyridin-3-yl)-3-(5-((4-(ethylsulfonyl)piperazin-1-yl)methyl)-2-fluorobenzyl)urea;
1-(5-((4-(ethylsulfonyl)piperazin-1-yl)methyl)-2-fluorobenzyl)-3-(6-methoxypyridin-3-yl)urea;
tert-butyl 4-(4-chloro-3-((3-(6-methylpyridin-3-yl)ureido)methyl)benzyl)piperazine-1-carboxylate;
1-(2-fluoro-3-((4-(isopropylsulfonyl)piperazin-1-yl)methyl)phenyl)-3-(6-methylpyridin-3-yl)urea;
1-(2-fluoro-3-((4-(propylsulfonyl)piperazin-1-yl)methyl)phenyl)-3-(6-methylpyridin-3-yl)urea;
1-(3-((4-(cyclopropylsulfonyl)piperazin-1-yl)methyl)-2-fluorophenyl)-3-(6-methylpyridin-3-yl)urea;
(R)-4-(2-fluoro-3-(3-(6-methylpyridin-3-yl)ureido)benzyl)-N,N,3-trimethylpiperazine-1-sulfonamide; and
1-(2-chloro-5-((4-(ethylsulfonyl)piperazin-1-yl)methyl)phenyl)-3-(6-methylpyridin-3-yl)urea.
